SEQUENCE LISTING (3 sequences)

SEQ ID NO: 1 - protein, 15 aa, Homo sapiens
TRYLRIHPQSWVHQI

SEQ ID NO: 2 - protein, 21 aa, Homo sapiens
  SITE 3: note = MISC_FEATURE - 4HYP 4-Hydroxyproline
  SITE 6: note = MISC_FEATURE - 4HYP 4-Hydroxyproline
  SITE 9: note = MISC_FEATURE - 4HYP 4-Hydroxyproline
  SITE 12: note = MISC_FEATURE - 4HYP 4-Hydroxyproline
  SITE 15: note = MISC_FEATURE - 4HYP 4-Hydroxyproline
  SITE 18: note = MISC_FEATURE - 4HYP 4-Hydroxyproline
  SITE 21: note = MISC_FEATURE - 4HYP 4-Hydroxyproline
GPXGPXGPXGPXGPXGPXGPX

SEQ ID NO: 3 - protein, 9 aa, Homo sapiens
CNPRGDYRC

(19) United States
(12) Patent Application Publication
(10) Pub. No.: US 2025/0134969 A1
(43) Pub. Date: May 1, 2025

(54) BIOSYNTHETIC HEMOSTAT AND USES THEREOF

(71) Applicant: CASE WESTERN RESERVE UNIVERSITY, Cleveland, OH (US)

(72) Inventors: Anirban Sen Gupta, Cleveland, OH (US); Aditya Girish, Cleveland Heights, OH (US)

(21) Appl. No.: 18/835,102

(22) PCT Filed: Feb. 2, 2023
(86) PCT No.: PCT/US2023/061850

§ 371 Date: Aug. 1, 2024

Related U.S. Application Data
(60) Provisional application No. 63/305,724, filed on Feb. 2, 2022.

(51) Int. Cl. A61K 38/48; A61K 9/127; A61K 38/36; A61K 47/24; A61K 47/64; A61K 47/69; A61P 7/04

(52) U.S. Cl. CPC A61K 38/4833; A61K 9/127; A61K 38/363; A61K 47/24; A61K 47/64; A61K 47/6911; A61P 7/04

(57) ABSTRACT

A biosynthetic hemostat includes a plurality of biocompatible flexible nanoparticles wherein each nanoparticle includes a shell that defines an outer surface of the nanoparticle and a core, which is loaded with thrombin, and a plurality of von Willebrand factor-binding peptides (VBPs) and collagen-binding peptides (CBPs) that are linked to the shell and extend from the outer surface.

### RELATED APPLICATION

This application claims priority from U.S. Provisional Application No. 63/305,724, filed Feb. 2, 2022, the subject matter of which is incorporated herein by reference in its entirety.

### SEQUENCE LISTING

The instant application contains a Sequence Listing which has been submitted electronically in XML format and is hereby incorporated by reference in its entirety. Said XML copy, created on Feb. 1, 2023, is named CWR-031343WO-ORD st.26 and is 6,420 bytes in size.

### BACKGROUND

Severe hemorrhage associated with trauma, surgery, congenital or drug-induced coagulopathies, etc. can be life-threatening and necessitates rapid hemostatic management via external/topical, intracavitary, and intravenous routes to reduce mortality risks. While bleeding from accessible and external injuries can be efficiently mitigated by tourniquets, bandages, pressure dressings, foams, and glues, options to manage internal and diffuse bleeding are limited to the transfusion of blood products (whole blood, blood components, concentrated are recombinant coagulation factors, etc.). The major hemostatic players in blood are platelets and coagulation factors, which through a complex concert of interactions and reactions localized at the injury site generate thrombin, which then converts fibrinogen to fibrin for hemostatic action. Therefore, the transfusion of platelets and coagulation factors (as plasma, prothrombin complex concentrate, cryoprecipitate, or individual recombinant factors) has become clinically significant in hemorrhage management. However, such products are donor dependent, are highly expensive, and often have a short shelf life due to contamination risks, which effect on-demand availability. Pathogen reduction technologies (PRTs) as well as reduced temperature processing and storage (cooling, freeze-drying, cryopreservation, etc.) are currently being studied for these products to improve their availability, portability, and transfusion logistics.

In parallel, significant research interest has emerged regarding the design of donor-independent biosynthetic intravenous hemostatic technologies using distinct biomaterials and nanotechnology approaches. Prominent recent examples of these are (i) “synthetic platelet” (SynthoPlate) nanoparticles that recapitulate platelets' hemostatically relevant binding interactions with von Willebrand factor (vWF), collagen, and active platelet integrin GPIIb-IIIa, (ii) platelet-like particles (PLPs) that bind fibrin to recapitulate platelets' hemostatically important biomechanical property of clot contraction, (iii) fibrinogen-function-mimicking nanoparticles that improve the aggregation of active platelets, (iv) tissue factor targeted injectable peptide amphiphiles, (v) a PolySTAT polymer that recapitulates factor FXIIIa function of fibrin stabilization and (vi) a HAPPI polymer that can accumulate at sites of high vWF and collagen exposure.

### SUMMARY

Embodiments described herein relate to a biosynthetic hemostat that includes a plurality of targeted thrombin-loaded biocompatible flexible nanoparticles and its use in treating trauma, surgery, congenital, and/or drug induced coagulopathies. The targeted thrombin-loaded biocompatible flexible nanoparticles can undergo peptide-ligand-mediated adhesion to von Willebrand factor (vWF) and collagen and can release thrombin at its site of adhesion after administration to a subject in need thereof. The biosynthetic hemostat demonstrated a promising ability to restore fibrin generation and clot characteristics in human plasma and blood when the natural hemostatic abilities were impaired by anticoagulation and platelet depletion. This ability of biosynthetic hemostat was also demonstrated with real-time imaging under a simulated vascular flow environment in a microfluidic setup. The in vivo safety and hemostatic efficacy of biosynthetic hemostat was established by its ability to significantly reduce bleeding in a tail-clip injury model in coagulopathic mice with prophylactic (preinjury) administration, as well as in a liver-laceration acute hemorrhagic injury model in coagulopathic mice with emergency (postinjury) administration. We show that the biosynthetic hemostat can directly deliver thrombin in a vascular-injury-site-targeted fashion, to enable site-localized fibrin generation for hemorrhage control.

In some embodiments, the targeted thrombin-loaded biocompatible flexible nanoparticles of the biosynthetic hemostat can each include a shell that defines an outer surface of the nanoparticle and a core, which is loaded with thrombin. A plurality of von Willebrand factor-binding peptides (VBPs) and collagen-binding peptides (CBPs) can be linked to the shell and extend from the outer surface. The nanoparticle is configured to adhere to a vascular surface, vascular disease site and/or vascular injury site with exposed von Willebrand factor and collagen, shield the loaded thrombin in circulation to avoid rapid inhibition or systemic thrombotic risk, and release the loaded thrombin at the vascular surface, vascular disease site and/or vascular injury site by diffusion from the nanoparticle and/or enzyme triggered degradation of the nanoparticle.

In some embodiments, the enzyme that triggers degradation of the nanoparticle is substantially unique or specific to a vascular injury site and/or has a higher concentration or activity compared to other cells, tissues, and/or disease sites in the subject. For example, the shell of the nanoparticle can include at least one phospholipid, and the enzyme can include at least on phospholipase that triggers phospholipase degradation of the at least one phospholipid and release of the thrombin. Other examples of enzymes that are substantially unique or specific to a vascular injury site and/or have a higher concentration or activity compared to other cells, tissues, and/or disease sites in the subject can include matrix metalloproteases (MMPs) or serine proteases, such as plasmin or neutrophil elastase.

In some embodiments, the nanoparticle binds to the vascular surface, vascular disease site and/or vascular injury site under a hemodynamic shear environment.

In some embodiments, the nanoparticle can have a shape, size and elastic modulus that facilitates margination to a vascular injury site upon administration to vasculature of a subject. For example, the nanoparticles can have an average or median diameter of about 50 nm to about 5 μm, preferably about 50 nm to about 200 nm, or more preferably about 100 nm to about 150 nm.

In some embodiments, the nanoparticle can be a liposome. The liposome can include a plurality of phospholipids and optionally cholesterol to define a lipid membrane.

In some embodiments, the phospholipids can include at least one of distearoylphosphatidylserine (DSPS), distearoylphosphatidylcholine dipalmitoylphosphatidylcholine (DSPC), dibehenoylglycerophosphocoline (DBPC), distearoylphosphatidylcholine (DSPC), diarachidonylphosphatidylcholine (DAPC), dioleoylphosphatidylethanolamine (DOPE), dipalmitoylphosphatidylethanolamine (DPPE), and distearoylphosphatidylethanolamine (DSPE); dipalmitoylphosphatidic acid (DPPA), or PEG functionalized lipids thereof.

In some embodiments, the VBPs and CBPs are spatially or topographically arranged on the outer surface such that the VBPs and CBPs do not spatially mask each other and the nanoparticle is able to adhere to a vascular surface, vascular disease site, and/or vascular injury site with exposed vWF and collagen and promote arrest and aggregation of active platelets onto sites of the nanoparticle adhesion.

In some embodiments, the VBPs and CBPs are conjugated to the phospholipids with PEG linkers. The VBP and CBP conjugated phospholipids can include about 1 mole % to about 10 mole %, preferably about 2.5 mole % to about 10 mole % of the total lipid composition of the liposome.

In some embodiments, the liposome includes DSPC, DSPE conjugated to VBP with PEG (DSPE-PEG-VBP), DSPE conjugated to CBP with PEG (DSPE-PEG-CBP), and cholesterol.

In some embodiments, the VBPs can have an amino acid sequence of SEQ ID NO: 1 and the CBPs can have an amino acid sequence of SEQ ID NO: 2. The ratio of VPB: CPB provided on the surface of the nanoparticle can be about 25:75 to about 75:25.

In other embodiments, the nanoparticle can further include a plurality of fibrinogen mimetic peptides (FMPs) that bind to GPIIb-IIIa, endothelial cell targeting peptides, and/or platelet targeting peptides that are linked to the shell and extend from the outer surface. For example, the FMPs can have an amino acid sequence of SEQ ID NO: 3.

In some embodiments, the thrombin loaded nanoparticles can provide vascular injury-site targeted delivery of thrombin to a subject. The amount of thrombin delivered to the vascular injury site in the subject by the hemostat is an amount effective to augment hemostasis in the subject.

In some embodiments, the nanoparticles can be further loaded with at least one of a platelet agonist, antifibrinolytic, coagulation factor, or prothrombin.

Other embodiments relate to the use of the biosynthetic hemostat described herein in a method of treating at least one of a trauma-induced coagulopathy, surgery-induced coagulopathy, congenital-induced coagulopathy, and/or drug-induced coagulopathy. For example, the biosynthetic hemostat can be used in a method of treating trauma-induced coagulopathy; congenital, disease associated, or drug induced hemostatic dysfunction; excessive bleeding; non-compressible and/or uncontrolled hemorrhage; or systemic bleeding dysfunction including polytrauma, internal bleeding, and/or a subject with platelet and coagulation factor defects.

In some embodiments, the hemostat can be administered systemically to the subject by, for example, parenteral administration including subcutaneous, intraperitoneal, intravenous, intradermal, or intramuscular administration.

In other embodiments, the hemostat can be administered locally by, for example, topical or intradermal administration.

In other embodiments, the hemostat can be administered to a site of vascular injury in a subject in need thereof to diminish bleeding in the subject.

In still other embodiments, the hemostat can be administered to a site of vascular injury in a subject to treat the vascular injury.

In some embodiments, the subject can have or be at increased risk of thrombocytopenia. The thrombocytopenia can be caused by or result from dehydration, leukemia, myelodysplastic syndrome, aplastic anemia, liver failure, sepsis, leptospirosis, congenital amegakaryocytic thrombocytopenia, thrombocytopenia absent radius syndrome, fanconi anemia, Bernard-Soulier syndrome, May-Hegglin anomaly, grey platelet syndrome, Alport syndrome, Wiskott-Aldrich syndrome, idiopathic thrombocytopeniaurpura, thrombotic thrombocytopenia purpura, hemolytic-uremic syndrome, disseminated intravascular coagulation, paroxysmal nocturnal hemoglobinuria, antiphospholipid syndrome, systemic lupus erythematosus, post-transfusion purpura, neonatal alloimmune thrombocytopenia, hypersplenism, dengue fever, Gaucher's disease, zika virus, medication-induced thrombocytopenia, niacin toxicity, Lyme disease, and thrombocytapheresis.

In some embodiments, administration of the hemostat can augment fibrin at the vascular surface, vascular disease site and/or vascular injury site for hemostatic effect, independent of native platelet depletion and/or dysfunction and/or platelet availability.

In still other embodiments, fibrinogen can be administered in combination with the hemostat.

### BRIEF DESCRIPTION OF THE DRAWINGS

FIGS. 1(A-B) illustrate a schematic showing platelet-mediated hemostatic mechanism and platelet-inspired t-TLNP design. (A) Platelets rapidly adhere at a vascular injury site by binding to von Willebrand factor (vWF, via platelet surface GPIbα) and collagen (via platelet surface GPIa/IIa and GPVI) exposed at the site and present high amounts of an anionic phospholipid such as phosphatidylserine (PS) on the activated platelet procoagulant membrane surface to enable the assembly of coagulation factors to form tenase (FVIIa+FIXa+FX) and prothrombinase (FXa+FVa+FII) complexes, ultimately leading to the amplified generation of thrombin (thrombin burst); the thrombin locally converts fibrinogen (Fg) to fibrin that gets cross-linked by FXIIIa for hemostatic clot formation. (B) t-TLNPs can undergo platelet-mimetic adhesion at the vascular injury site by anchoring to vWF via vWF-binding peptide (VBP) and collagen via collagen-binding peptide (CBP) and release thrombin at the site via diffusion as well as injury site secreted phospholipase A₂ (sPLA₂) triggered particle destabilization; this thrombin can locally convert fibrinogen (Fg) to fibrin for hemostatic action.

FIGS. 2(A-G) illustrate the manufacture and characterization of t-TLNPs. (A) Bioconjugation schematics of reacting cysteine-terminated peptides to maleimide-terminated DSPE-PEG_{2K} utilizing thiol-maleimide chemistry to synthesize DSPE-PEG_{2K} -peptide molecules. (B) Molecular components of t-TLNP manufacture. (C) Dynamic light scattering (DLS) analysis of five representative t-TLNP batches showing nanoparticle size reproducibility. (D) Cryo-transmission electron microscopy (Cryo-TEM) images of t-TLNPs (scale bar: 100 nm) showing a particle diameter of about 175 nm. (E) Representative images from BioFlux experiments where Rhodamine B labeled control (undecorated) vs targeted nanoparticles (“VBP+CBP”-decorated) were flowed at 25 dyn/cm² over “vWF+collagen”-coated channels and targeted nanoparticles were also flowed over albumin-coated channels, showing substantially high adhesion of targeted nanoparticles to “vWF+collagen”-coated surface but not of control particles to the “vWF+collagen”-coated surface or targeted nanoparticles to the albumin-coated surface. (F) Spectrometric analysis of three representative t-TLNP batches showing a mean thrombin loading of 114.3±14.2 nM. (G) Thrombin release analysis showing that t-TLNPs can slowly release low amounts of thrombin by diffusion, whereas exposure to sPLA₂ significantly enhances thrombin release. *p≤0.05, **p≤0.01.

FIGS. 3(A-C) illustrate the evaluation of biosafety characteristics of peptide-decorated nanoparticles. (A) Monolayers of healthy human pulmonary microvascular endothelial cells (HPMEC, nuclei stained with blue DAPI) were exposed to TNF-α (a known endothelial activator), media only, control (undecorated) nanoparticles or targeted (“VBP+CBP”-decorated) nanoparticles and vWF expression on endothelium was stained (green vWF antibody) as a marker for endothelial activation. In comparison to TNF-α-induced stimulation (high vWF staining), neither control particles nor targeted particles showed endothelial activation (low vWF staining, similar to that of the “media only” group). (B) Platelet lumi-aggregometry studies with human platelet-rich plasma (PRP) showed that addition of ADP (platelet agonist) induced significant platelet aggregation but addition of the targeted nanoparticles (t-LNPs) did not induce such aggregation (aggregation percent similar to that of the “no ADP” group); (C) ELISA-based complement C3 activation assay studies using human plasma incubated with control nanoparticles or targeted nanoparticles (t-LNPs) indicated that neither control nor targeted nanoparticles activate C3 (C3a/C3 ratio similar to the baseline of saline-incubated plasma).

FIGS. 4(A-C) illustrate the evaluation of t-TLNPs in restoring fibrin generation in anticoagulated and platelet-depleted human plasma. (A) Schematic of the experimental design where human whole blood (WB) was centrifuged to obtain platelet-rich plasma (PRP) and the PRP was further centrifuged to obtain either platelet-poor plasma (PPP) or platelet-free plasma (PFP); The PPP was treated with anticoagulant Apixaban (FXa inhibitor). PFP and Apixaban-treated PPP were both subjected to spectrophotometric monitoring of fibrin generation (measuring optical density of formed/polymerized fibrin over time at 405 nm) and the onset of fibrin generation (OFG), maximum optical density (also called maximum hemostatic potential or MHP) and area under the curve (also called overall coagulation potential or OCP) was recorded. (B1-B3) Effect of adding t-TLNP vs UNP in Apixaban-treated PPP, demonstrating that thrombin released by t-TLNP can restore OCP, OFG, and MHP parameters closer to the normal plasma baseline (increased OCP, reduced OFG, increased MHP) and this effect is enhanced when sPLA2 is added to accelerate thrombin release. (C1-C3) Effect of adding t-TLNP vs UNP in PFP, demonstrating that thrombin released by t-TLNP can restore OCP, OFG, and MHP parameters closer to the normal plasma baseline and this effect is enhanced when sPLA2 is added to accelerate thrombin release. *p≤0.05, **p≤0.01, ***p≤0.001, and ****p≤0.0001.

FIGS. 5(A-C) illustrate the evaluation of t-TLNPs in restoring clot viscoelastic parameters as measured by rotational thromboelastometry (ROTEM). (A) Schematic of the experimental design where human whole blood (WB) was directly treated with the anticoagulant Apixaban (FXa inhibitor) or was fractionated into blood components (RBC, platelets, leukocytes, plasma) and then reconstituted with a reduced number of platelets to create thrombocytopeniale blood (TC Blood). Anticoagulated blood and thrombocytopenialood were analyzed in ROTEM in NATEM mode (CaCl₂-induced blood clotting resisting pin rotation), and the clot formation time (CFT), clot formation rate (also called alpha angle), and early clot amplitude at 10 min (also called A10) were monitored. (B1-B3) Effect of adding t-TLNP vs UNP in Apixaban-treated WB, demonstrating that thrombin released by t-TLNP can significantly restore CFT, alpha angle, and A10 parameters closer to the normal WB baseline (reduced CFT, increased alpha angle, increased A10) and this effect is enhanced when sPLA₂ is added to accelerate thrombin release. (C1-C3) Effect of adding t-TLNP vs UNP in thrombocytopenialood (TC Blood) demonstrating that thrombin released by t-TLNP can partially restore CFT, alpha angle, and A10 parameters closer to the normal WB baseline, but no statistical significance was observed in this improvement without or with sPLA₂. *p≤0.05, **p≤0.01, ***p≤0.001, and ****p≤0.0001.

FIGS. 6(A-E) illustrate the evaluation of t-TLNPs in restoring fibrin generation under a simulated vascular flow environment in human plasma containing the combined hemostatic defect of platelet depletion plus anticoagulation. (A) Schematic of the BioFlux microfluidic setup and experimental design where human plasma containing fluorescently labeled platelets and fibrinogen (by Calcein and AlexaFluor647, respectively) were flowed over “vWF+collagen”-coated microchannel and fibrin formation was imaged in real time. (B) Representative fluorescence images of fibrin formation over time (0-12 min) in the microchannel with flows of platelet-rich plasma (PRP), platelet-depleted plus anticoagulated (Defective) plasma, Defective plasma treated with “t-TLNPs+sPLA₂” and Defective plasma treated with “UNPs+sPLA₂” showing that the combined defect of platelet depletion plus anticoagulation in plasma drastically reduces fibrin formation in comparison to that in PRP and treatment with “t-TLNPs+sPLA₂” is able to restore fibrin generation even when platelet numbers were low. Treatment with “UNP+sPLA₂” was unable to restore fibrin. (C) Representative dual-fluorescence images of the full microchannel surface at the experiment end point (12 min) showing a substantial number of blue platelets enmeshed in green fibrin in the channel containing PRP flow. In comparison, the channel with Defective plasma showed sparse platelets and minimal fibrin and Defective plasma treated with “t-TLNPs+sPLA₂” showed fibrin recovery even though the platelets were sparse, while treatment with “UNPs+sPLA₂” showed no such recovery. (D) Surface-averaged fluorescence intensity quantification of fibrin corroborating that treatment of Defective plasma with “t-TLNP+sPLA₂” restores fibrin generation comparably to that of PRP. (E) D-dimer ELISA based quantification of digested fibrin from the microchannels further confirming that treatment of Defective plasma with “t-TLNP+sPLA₂” restores the formation of cross-linked fibrin at concentrations comparable to those of PRP. *p≤0.05, **p≤0.01, ***p≤0.001.

FIGS. 7(A-C) illustrate the evaluation of prophylactic administration of t-TLNPs in restoring hemostatic efficacy in the tail-clip model in mice with significant bleeding due to the combined effect of platelet depletion and anticoagulation. (A) Schematic of the experimental design where mice were first made thrombocytopeniaC Mouse) by anti-CD42b dose induced platelet clearance and then further dosed with anticoagulant (Enoxaparin) to induce combine a hemostatic defect (“Defect” mouse). t-TLNP or UNP treatment was administered in the “Defect” mice via an intravenous (retroorbital) route and allowed to circulate for 15 min, and then a tail-clip injury was performed to measure the bleeding time and blood loss. (B) Bleeding time data as a percent of 15 min time period showing that normal mice stopped bleeding in 3.11±0.44 min while the combined effect of thrombocytopenia and anticoagulation in “Defect” mice resulted in continuous bleeding for 15 min (and beyond). Treatment of t-TLNPs in defect mice significantly restores the hemostatic capability, with the mice stopping bleeding in 6.18±3.19 min, while treatment with UNPs has no such effect (mice continue bleeding for 15 min and beyond). (C) Blood loss analysis via a spectrophotometric measurement of hemoglobin in shed blood indicating that the combined effect of thrombocytopenia and anticoagulation in “Defect” mice results in significantly increased blood loss over the 15 min time period in comparison to normal mice. Treatment of “Defect” mice with t-TLNPs significantly reduces blood loss. In contrast, treatment with UNPs did not reduce blood loss but rather exacerbated it, possibly due to a dilution effect. *≤0.05, **p≤0.01, ***p≤0.001.

FIGS. 8(A-B) illustrate the evaluation of emergency administration of t-TLNPs in restoring hemostatic efficacy in liver laceration bleeding model in anticoagulated mice. (A) Schematic and representative anatomic picture of liver laceration model in mice where treatment (sham saline, UNP, or t-TLNP) was administered post-injury and blood loss from the injured liver was measured by preweighed gauze. (B) Blood loss data (in grams, g) from mouse liver injury model studies showing a significant increase in bleeding from an injured liver in defect (heparinized) mice in comparison to normal (nonheparinized) mice. Treatment with UNPs was unable to reduce blood loss, but treatment with t-TLNPs was able to significantly reduce blood loss. *p≤0.05, **p≤0.01, ***p≤0.001, ****p≤0.0001.

FIGS. 9(A-B) illustrate a bioconjugation reaction scheme and representative mass spectroscopy characterization data for DSPE-PEG-VBP and DSPE-PEG-CBP.

FIGS. 10(A-B) illustrate (A) Schematic of BioFlux microfluidic set-up for nanoparticle adhesion studies; (B) Representative fluorescence images of Rhodamine-B labeled (red fluorescent) control nanoparticles (no peptide decoration) vs. targeted nanoparticles (‘VBP+CBP’-decorated) binding to ‘vWF+collagen’-coated surface pre- and post-incubation in human platelet-free plasma.

FIG. 11 illustrates carboxyfluorescein (CF) concentration vs. fluorescence calibration curve, and CF release levels from t-TLNPs via diffusion vs. sPLA2-triggered particle degradation vs. chloroform/methanol-induced exhaustive destabilization vs. exposure to low-to-high shear stress.

FIGS. 12(A-C) illustrate (A) Representative aggregometry profile of human platelet-rich plasma (PRP) at baseline (no ADP), vs. with agonist (ADP) or free VBP or free CBP; (B) Representative aggregometry profile of human platelet-rich plasma (PRP) at baseline (no ADP), vs. with agonist (ADP) or t-LNP; (C) Representative fluorescent images of neutrophils (DAPI: blue nuclei, Sytox Green: extracellularized DNA NETs) at baseline (w/o stimulation) compared to neutrophils incubated with control nanoparticles (no peptide decoration), or targeted nanoparticles (‘VBP+CBP’-decorated), or calcium ionophore A23187, and quantitative data of Sytox Green fluorescence comparing these groups.

FIGS. 13(A-B) illustrate representative Optical Density (OD) data for fibrin generation in plasma, plasma anticoagulated with Apixaban, and anticoagulated plasma treated with t-TLNPs, ‘t-TLNPs+sPLA2’ and UNPs.

FIGS. 14(A-B) illustrate representative OD data for fibrin generation in platelet-rich plasma (PRP), platelet-free plasma (PFP), and PFP treated with t-TLNPs, ‘t-TLNPs+sPLA2’ and UNPs.

FIGS. 15(A-B) illustrate (A) Representative OD data for fibrin generation in plasma, plasma anticoagulated with Apixaban, and this anticoagulated plasma treated with directly added thrombin; (B) Representative OD data for fibrin generation in platelet-rich plasma (PRP), platelet-free plasma (PFP), and this PFP treated with directly added thrombin.

FIGS. 16(A-B) illustrate (A) Representative OD data for fibrin generation in plasma, plasma anticoagulated with Apixaban, and this anticoagulated plasma treated with various doses of t-TLNPs (hence various concentrations of encapsulated thrombin); (B) Representative Optical Density (OD) data for fibrin generation in platelet-rich plasma (PRP), platelet-free plasma (PFP), and this PFP treated with various doses of t-TLNPs (hence various concentrations of encapsulated thrombin).

FIGS. 17(A-B) illustrate representative ROTEM data showing debilitation of CFT, alpha Angle and A10 parameters when WB is anticoagulated with Apixaban, and corresponding effects of treatment with t-TLNPs, ‘t-TLNPs+sPLA2’ and UNPs.

FIGS. 18(A-B) illustrate Representative ROTEM data showing debilitation of CFT, alpha Angle and A10 parameters when platelets are depleted from WB to make TC Blood, and corresponding effects of treatment with t-TLNPs, ‘t-TLNPs+sPLA2’ and UNPs.

FIGS. 19(A-C) illustrate the effect of anti-CD42b antibody dose on platelet count in mice showing significant thrombocytopenia (TC) and bleeding time analysis in tail-clip injury model in TC mice compared to wild type (WT) normal mice.

FIG. 20 illustrates Circulation lifetime characterization of t-TLNPs in mice and organ biodistribution studies of t-TLNPs in mice over 24 hr period.

FIG. 21 illustrates tail-clip injury bleeding time data in WT mice vs. coagulopathic mice (thrombocytopenia plus anticoagulation) and effect of t-LNP treatment vs. UNP treatment on bleeding time in this coagulopathic mice represented in a Kaplan-Meyer format.

FIG. 22 illustrates mean arterial pressure (MAP) characterization of mice subjected to liver laceration injury and observed for over 20 min period post-injury before retrieval of absorbent triangles.

FIG. 23 illustrates representative histology images (Carstairs' staining) of uninjured liver section and clearance organs harvested from mice subjected to liver injury model, post-injury treatment with saline or UNP or t-TLNP, and euthanasia at 3-hr time-point post-injury.

### DETAILED DESCRIPTION

All scientific and technical terms used in this application have meanings commonly used in the art unless otherwise specified. The definitions provided herein are to facilitate understanding of certain terms used frequently herein and are not meant to limit the scope of the application.

The articles “a” and “an” are used herein to refer to one or to more than one (i.e., to at least one) of the grammatical object of the article. By way of example, “an element” means one element or more than one element.

The term “about” will be understood by persons of ordinary skill in the art and will vary to some extent on the context in which it is used.

As used herein, the term “subject” can refer to any animal including, but not limited to, humans and non-human animals (e.g., rodents, arthropods, insects, fish (e.g., zebrafish)), non-human primates, ovines, bovines, ruminants, lagomorphs, porcines, caprines, equines, or canines felines, aves, etc.).

The terms “diminishing,” “reducing,” or “preventing,” “inhibiting,” and variations of these terms, as used herein include any measurable decrease, including complete or substantially complete inhibition. The terms “enhance” or “enhanced” as used herein include any measurable increase or intensification.

As used herein, the term “small molecule” can refer to lipids, carbohydrates, polynucleotides, polypeptides, or any other organic or inorganic molecules.

As used herein, the term “polypeptide” refers to a polymer composed of amino acid residues related naturally occurring structural variants, and synthetic non-naturally occurring analogs thereof linked via peptide bonds or modified peptide bonds (i.e., peptide isosteres), related naturally occurring structural variants, and synthetic non-naturally occurring analogs thereof, glycosylated polypeptides, and all “mimetic” and “peptidomimetic” polypeptide forms. Synthetic polypeptides can be synthesized, for example, using an automated polypeptide synthesizer. The term can refer to an oligopeptide, peptide, polypeptide, or protein sequence, or to a fragment, portion, or subunit of any of these. The term “protein” typically refers to large polypeptides. The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus.

A “portion” of a polypeptide means at least about three sequential amino acid residues of the polypeptide. It is understood that a portion of a polypeptide may include every amino acid residue of the polypeptide.

“Mutants,” “derivatives,” and “variants” of a polypeptide (or of the DNA encoding the same) are polypeptides which may be modified or altered in one or more amino acids (or in one or more nucleotides) such that the peptide (or the nucleic acid) is not identical to the wild-type sequence, but has homology to the wild type polypeptide (or the nucleic acid).

A “mutation” of a polypeptide (or of the DNA encoding the same) is a modification or alteration of one or more amino acids (or in one or more nucleotides) such that the peptide (or nucleic acid) is not identical to the sequences recited herein, but has homology to the wild type polypeptide (or the nucleic acid).

As used herein, the term “targeting moiety” can refer to a molecule or molecules that are able to bind to and complex with a biomarker. The term can also refer to a functional group that serves to target or direct a therapeutic agent to a particular location, cell type, diseased tissue, or association. In general, a “targeting moiety” can be directed against a biomarker.

An “effective amount” can refer to that amount of a therapeutic agent that results in amelioration of symptoms or a prolongation of survival in the subject and relieves, to some extent, one or more symptoms of the disease or returns to normal (either partially or completely) one or more physiological or biochemical parameters associated with or causative of the disease. “Therapeutic agents” can include any agent (e.g., molecule, drug, pharmaceutical composition, etc.) capable of be encapsulated by or conjugated to a nanoparticle or microparticle construct of the application and further capable of preventing, inhibiting, or arresting the symptoms and/or progression of a disease.

“Nanoparticle” or “microparticle” as used herein is meant to include particles, spheres, capsules, and other structures having a length or diameter of about 10 nm to about 100 μm. For the purposes of this application, the terms “nanosphere”, “nanoparticle”, “nanoparticle construct”, “nanovehicle”, “nanocapsule”, “microsphere”, “microparticle”, and “microcapsule” are used interchangeably.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual and partial numbers within that range, for example, 1, 2, 3, 4, 5, 5.5 and 6. This applies regardless of the breadth of the range.

Embodiments described herein relate to a biosynthetic hemostat that includes a plurality of targeted thrombin-loaded biocompatible flexible nanoparticles and its use in treating trauma, surgery, congenital, and/or drug induced coagulopathies. The targeted thrombin-loaded biocompatible flexible nanoparticles can undergo peptide-ligand-mediated adhesion to von Willebrand factor (vWF) and collagen and can release thrombin at its site of adhesion after administration to a subject in need thereof. The biosynthetic hemostat demonstrated a promising ability to restore fibrin generation and clot characteristics in human plasma and blood when the natural hemostatic abilities were impaired by anticoagulation and platelet depletion. This ability of biosynthetic hemostat was also demonstrated with real-time imaging under a simulated vascular flow environment in a microfluidic setup. The in vivo safety and hemostatic efficacy of biosynthetic hemostat was established by its ability to significantly reduce bleeding in a tail-clip injury model in coagulopathic mice with prophylactic (preinjury) administration, as well as in a liver-laceration acute hemorrhagic injury model in coagulopathic mice with emergency (postinjury) administration. We show that the biosynthetic hemostat can directly deliver thrombin in a vascular-injury-site-targeted fashion, to enable site-localized fibrin generation for hemorrhage control.

It is therefore an aspect of the application that administration, such as by intravenous or topical administration, of the biosynthetic hemostat described herein to a subject with a vascular injury can diminish the bleeding time in the subject, provide a nanostructure that binds with a vascular injury site, and aid in stopping bleeding and particularly hemorrhage from trauma, surgery, congenital, and/or drug induced coagulopathies.

In some embodiments, the targeted thrombin-loaded biocompatible flexible nanoparticles of the biosynthetic hemostat can each include a shell that defines an outer surface of the nanoparticle and a core, which is loaded with thrombin. A plurality of von Willebrand factor-binding peptides (VBPs) and collagen-binding peptides (CBPs) can be linked to the shell and extend from the outer surface. The nanoparticle is configured to adhere to a vascular surface, vascular disease site and/or vascular injury site with exposed von Willebrand factor and collagen, shield the loaded thrombin in circulation to avoid rapid thrombin inhibition or systemic thrombotic risk, and release the loaded thrombin at the vascular surface, vascular disease site and/or vascular injury site by diffusion from the nanoparticle and/or enzyme triggered degradation of the nanoparticle.

In some embodiments, the enzyme that triggers degradation of the nanoparticle is substantially unique or specific to a vascular injury site and/or has a higher concentration or activity compared to other cells, tissues, and/or disease sites in the subject. For example, the shell of the nanoparticle can include at least one phospholipid, and the enzyme can include at least on phospholipase that triggers phospholipase degradation of the at least one phospholipid and release of the thrombin. Other examples of enzymes that are substantially unique or specific to a vascular injury site and/or have a higher concentration or activity compared to other cells, tissues, and/or disease sites in the subject can include matrix metalloproteases (MMPs) or serine proteases, such as plasmin or neutrophil elastase.

The biocompatible, biodegradable, flexible nanoparticles can be made from any biocompatible, biodegradable material that can form a flexible nanoparticle to which the peptides described herein can be attached, conjugated, and/or decorated and which can be loaded with thrombin prior to administration to a subject, shield the thrombin in circulation to avoid rapid inhibition or systemic thrombotic risk, and release the thrombin at the vascular surface, vascular disease site and/or vascular injury site by diffusion from the nanoparticle and/or enzyme triggered degradation of the nanoparticle. The biodegradable material can form an outer shell that can prevent the thrombin from reacting with fibrinogen in the subject until the nanoparticles adhere to a vascular surface, vascular disease site and/or vascular injury site. In some embodiments, the biocompatible, biodegradable flexible nanoparticles can include a liposome, lipidic nanoparticles, dendrimers, a hydrogel, micelle, polymer, and/or a combination of these materials that can include and/or be surface modified or engineered with the VBPs and CBPs, loaded with thrombin, and release the thrombin by diffusion from the nanoparticle and/or enzyme triggered degradation of the nanoparticle.

In some embodiments, the nanoparticle can have a shape, size and elastic modulus that facilitates margination to a vascular injury site upon administration to vasculature of a subject. For example, the nanoparticles can have an average or median diameter of about 50 nm to about 5 μm, preferably about 50 nm to about 200 nm, or more preferably about 100 nm to about 150 nm. In general, the nanoparticle construct can have dimensions small enough to allow the hemostat to be systemically administered to a subject and targeted to cells, tissue, and/or disease sites of the subject. In some embodiments, the nanoparticle construct can have a size that facilitates encapsulation of the thrombin and optionally one or more therapeutic and/or imaging agents.

In some embodiments, the nanoparticle binds to the vascular surface, vascular disease site and/or vascular injury site under a hemodynamic shear environment, preferably, under flow of about 5 to about 60 dynes/cm².

The nanoparticles of the biosynthetic hemostat may be uniform (e.g., being about the same size) or of variable size. Particles may be any shape (e.g., spherical or rod shaped), but are preferably made of regularly shaped material (e.g., spherical). Other geometries can include substantially spherical, circular, triangle, quasi-triangle, square, rectangular, hexagonal, oval, elliptical, rectangular with semi-circles or triangles and the like.

In some embodiments, the nanoparticles can include lipidic nanoparticles, polymer nanoparticles, liposomes, and/or dendrimers with a membrane, shell, or surface. The lipidic nanoparticles, polymer nanoparticles, liposomes, and/or dendrimers can be formed from naturally-occurring, synthetic or semi-synthetic (i.e., modified natural) materials that can loaded with thrombin, and release the thrombin by diffusion from the nanoparticle and/or enzyme triggered degradation of the nanoparticle.

In some embodiments, the lipidic nanoparticles or liposomes can include a membrane or shell that is formed from a naturally-occurring, synthetic or semi-synthetic material that is generally amphipathic (i.e., including a hydrophilic component and a hydrophobic component). Examples of materials that can be used to form the membrane or shell of the lipidic nanoparticle or liposome include lipids, such as fatty acids, neutral fats, phospholipids, oils, glycolipids, surfactants, cholesterol, aliphatic alcohols, waxes, terpenes and steroids, as well as semi-synthetic or modified natural lipids. Semi-synthetic or modified natural lipids can include natural lipids that have been chemically modified in some fashion. The lipid can be neutrally-charged, negatively-charged (i.e., anionic), or positively-charged (i.e., cationic). Examples of anionic lipids can include phosphatidic acid, phosphatidyl glycerol, and fatty acid esters thereof, amides of phosphatidyl ethanolamine, such as anandamides and methanandamides, phosphatidyl inositol and fatty acid esters thereof, cardiolipin, phosphatidyl ethylene glycol, acidic lysolipids, sulfolipids and sulfatides, free fatty acids, both saturated and unsaturated, and negatively-charged derivatives thereof. Examples of cationic lipids can include N-[1-(2,3-dioleoyloxy)propyl]-N,N,N-trimethyl-ammonium chloride and common natural lipids derivatized to contain one or more basic functional groups.

Other examples of lipids, any one or combination of which may be used to form the membrane or shell of the lipidic nanoparticle or liposome can include: phosphocholines, such as 1-alkyl-2-acetoyl-sn-glycero 3-phosphocholines, and 1-alkyl-2-hydroxy-sn-glycero 3-phosphocholines; phosphatidylcholine with both saturated and unsaturated lipids, including dioleoylphosphatidylcholine, dimyristoylphosphatidylcholine, dipentadecanoylphosphatidylcholine, dilauroylphosphatidylcholine, distearoylphosphatidylserine (DSPS), dipalmitoylphosphatidylcholine (DPPC), distearoylphosphatidylcholine (DSPC), and diarachidonylphosphatidylcholine (DAPC); phosphatidylethanolamines, such as dioleoylphosphatidylethanolamine, dipalmitoylphosphatidylethanolamine (DPPE), and distearoylphosphatidylethanolamine (DSPE); phosphatidylglycerols, including distearoylphosphatidylglycerol (DSPG); phosphatidylinositol; sphingolipids, such as sphingomyelin; glycolipids, such as ganglioside GM1 and GM2; glucolipids; sulfatides; glycosphingolipids; phosphatidic acids, such as dipalmitoylphosphatidic acid (DPPA) and distearoylphosphatidic acid (DSPA); palmitic acid; stearic acid; arachidonic acid; oleic acid; lipids bearing polymers, such as chitin, hyaluronic acid, polyvinylpyrrolidone or polyethylene glycol (PEG); lipids bearing sulfonated mono-, di-, oligo- or polysaccharides; cholesterol, cholesterol sulfate, and cholesterol hemisuccinate; tocopherol hemisuccinate; lipids with ether and ester-linked fatty acids; polymerized lipids (a wide variety of which are well known in the art); diacetyl phosphate; dicetyl phosphate; stearylaamine; cardiolipin; phospholipids with short chain fatty acids of about 6 to about 8 carbons in length; synthetic phospholipids with asymmetric acyl chains, such as, for example, one acyl chain of about 6 carbons and another acyl chain of about 12 carbons; ceramides; non-ionic liposomes including niosomes, such as polyoxyalkylene (e.g., polyoxyethylene) fatty acid esters, polyoxyalkylene (e.g., polyoxyethylene) fatty alcohols, polyoxyalkylene (e.g., polyoxyethylene) fatty alcohol ethers, polyoxyalkylene (e.g., polyoxyethylene) sorbitan fatty acid esters (e.g., the class of compounds referred to as TWEEN (commercially available from ICI Americas, Inc., Wilmington, DE), glycerol polyethylene glycol oxystearate, glycerol polyethylene glycol ricinoleate, alkyloxylated (e.g., ethoxylated) soybean sterols, alkyloxylated (e.g., ethoxylated) castor oil, polyoxyethylene-polyoxypropylene polymers, and polyoxyalkylene (e.g., polyoxyethylene) fatty acid stearates; sterol aliphatic acid esters including cholesterol sulfate, cholesterol butyrate, cholesterol isobutyrate, cholesterol palmitate, cholesterol stearate, lanosterol acetate, ergosterol palmitate, and phytosterol n-butyrate; sterol esters of sugar acids including cholesterol glucuronide, lanosterol glucuronide, 7-dehydrocholesterol glucuronide, ergosterol glucuronide, cholesterol gluconate, lanosterol gluconate, and ergosterol gluconate; esters of sugar acids and alcohols including lauryl glucuronide, stearoyl glucuronide, myristoyl glucuronide, lauryl gluconate, myristoyl gluconate, and stearoyl gluconate; esters of sugars and aliphatic acids including sucrose laurate, fructose laurate, sucrose palmitate, sucrose stearate, glucuronic acid, gluconic acid and polyuronic acid; saponins including sarsasapogenin, smilagenin, hederagenin, oleanolic acid, and digitoxigenin; glycerol dilaurate, glycerol trilaurate, glycerol dipalmitate, glycerol and glycerol esters including glycerol tripalmitate, glycerol distearate, glycerol tristearate, glycerol dimyristate, glycerol trimyristate; long chain alcohols including n-decyl alcohol, lauryl alcohol, myristyl alcohol, cetyl alcohol, and n-octadecyl alcohol; 6-(5-cholesten-3β-yloxy)-1-thio-β-D-galactopyranoside; digalactosyldiglyceride; 6-(5-cholesten-3β-yloxy)hexyl-6-amino-6-deoxy-1-thio-β-D-galactopyranoside; 6-(5-cholesten-3β-yloxy)hexyl-6-amino-6-deoxyl-1-thio-α-D-mannopyranoside; 12-(((7′-diethylaminocoumarin-3-yl)carbonyl)methylamino)octadecanoic acid; N-[12-(((7′-diethylaminocoumarin-3-yl)carbonyl)methylamino)octadecanoyl]-2-aminopalmitic acid; cholesteryl (4′-trimethylammonio)butanoate; N-succinyldioleoylphosphatidylethanolamine; 1,2-dioleoyl-sn-glycerol; 1,2-dipalmitoyl-sn-3-succinylglycerol; 1,3-dipalmitoyl-2-succinylglycerol; 1-hexadecyl-2-palmitoylglycerophosphoethanolamine and palmitoylhomocysteine; and/or any combinations thereof.

Examples of biocompatible, biodegradable polymers that can be used to form the nanoparticles are poly(lactide)s, poly(glycolide)s, poly(lactide-co-glycolide)s, poly(lactic acid)s, poly(glycolic acid)s, poly(lactic acid-co-glycolic acid)s, polycaprolactone, polycarbonates, polyesteramides, polyanhydrides, poly(amino acids), polyorthoesters, polyacetyls, polycyanoacrylates, polyetheresters, poly(dioxanone)s, poly(alkylene alkylate)s, copolymers of polyethylene glycol and poly(lactide)s or poly(lactide-co-glycolide)s, biodegradable polyurethanes, and blends and/or copolymers thereof.

Other examples of materials that may be used to form the nanoparticles or microparticles cn include chitosan, poly(ethylene oxide), poly(lactic acid), poly(acrylic acid), poly(vinyl alcohol), poly(urethane), poly(N-isopropyl acrylamide), poly(vinyl pyrrolidone) (PVP), poly(methacrylic acid), poly(p-styrene carboxylic acid), poly(p-styrenesulfonic acid), poly(vinylsulfonicacid), poly(ethyleneimine), poly(vinylamine), poly(anhydride), poly(L-lysine), poly(L-glutamic acid), poly(gamma-glutamic acid), poly(carprolactone), polylactide, poly(ethylene), poly(propylene), poly(glycolide), poly(lactide-co-glycolide), poly(amide), poly(hydroxylacid), poly(sulfone), poly(amine), poly(saccharide), poly(HEMA), poly(anhydride), gelatin, glycosaminoglycans (GAG), poly(hyaluronic acid), poly(sodium alginate), alginate, albumin, hyaluronan, agarose, polyhydroxybutyrate (PHB), copolymers thereof, and blends thereof.

In some embodiments, the nanoparticle can be liposomes. The liposomes can include a plurality of phospholipids and optionally cholesterol to define a lipid membrane. The phospholipids can include at least one of distearoylphosphatidylserine (DSPS), distearoylphosphatidylcholine (DSPC), dipalmitoylphosphatidylcholine (DPPC), dibehenoylglycerophosphocoline (DBPC), distearoylphosphatidylcholine (DSPC), diarachidonylphosphatidylcholine (DAPC), dioleoylphosphatidylethanolamine (DOPE), dipalmitoylphosphatidylethanolamine (DPPE), and distearoylphosphatidylethanolamine (DSPE); dipalmitoylphosphatidic acid (DPPA), or PEG functionalized lipids thereof.

In some embodiments, the lipid membrane can include at least one phospholipid that is amenable to degradation by injury site specific enzymes, such as phospholipase degradation. For example, distearoylphosphatidylcholine (DSPC) can be used as one of the lipid components, since DSPC is amenable to degradation by injury site specific enzyme sPLA2. Other examples of enzymes that are substantially unique or specific to a vascular injury site and/or have a higher concentration or activity compared to other cells, tissues, and/or disease sites in the subject can include matrix metalloproteases (MMPs) or serine proteases, such as plasmin or neutrophil elastase.

In one example, the nanoparticle can be liposome that includes distearoylphosphatidylcholine (DSPC). The DSPC can be provided in the lipid membrane at about 10 mole % to about 50 mole %, about 15 mole % to about 45 mole %, or about 20 mole % to about 40 mole % of the lipid membrane.

In some embodiments, the liposome can be an unilamellar liposome and can have a width or diameter less than about 200 nm. For example, the width or diameter of the liposome can be about 100 nm to about 150 nm. In some embodiments, the liposome is about 150 nm in diameter. The liposome can have a high cholesterol content (e.g., at least about 20 mole %) in the membrane in order to efficiently encapsulate the thrombin protecting the thrombin from plasma deactivation in circulation and prevent premature thrombin leakage due to membrane rigidity.

In certain embodiments, the VBPs and CBPs specifically bind to respectively, exposed, vWF and collagen at a vascular surface, vascular disease site, and/or vascular injury site. As used herein, the VBPs and CBPs “specifically bind” to a vWF and collagen if they bind to or associate with the vWF and collagen with an affinity or Ka (that is, an equilibrium association constant of a particular binding interaction with units of 1/M) of, for example, greater than or equal to about 10⁵ M⁻¹. In certain embodiments, the VBPs and CBPs bind to the vWF and collagen with a Ka greater than or equal to about 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, or 10¹³ M⁻¹. “High affinity” binding refers to binding with a Ka of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, at least 10¹³ M⁻¹, or greater. Alternatively, affinity may be defined as an equilibrium dissociation constant (KD) of a particular binding interaction with units of M (e.g., 10⁻⁵ M to 10⁻¹³ M, or less). In certain aspects, specific binding means binding to the vWF and collagen with a KD of less than or equal to about 10⁻⁵ M, less than or equal to about 10⁻⁶ M, less than or equal to about 10⁻⁷ M, less than or equal to about 10⁻⁸ M, or less than or equal to about 10⁻⁹ M, 10⁻¹⁰ M, 10⁻¹¹ M, or 10⁻¹² M or less.

In some embodiments, the VBPs and CBPs are spatially or topographically arranged on the outer surface such that the VBPs and CBPs do not spatially mask each other and the nanoparticle is able to adhere to a vascular surface, vascular disease site, and/or vascular injury site with exposed vWF and collagen and promote arrest and aggregation of active platelets onto sites of the nanoparticle adhesion.

In some embodiments, the VBP peptide for vWF binding can include a recombinant GPIbα fragment (rGPIbα) containing the vWF binding sites (residues 1 to 302) or a short chain vWF-binding peptide. The GPIbα fragment can be expressed in CHO cells and isolated, adapting methods described. The short vWF-binding peptide can include the amino acid sequence of TRYLRIHPQSWVHQI (SEQ ID NO: 1). A peptide having an amino acid sequence of SEQ ID NO: 1 can be synthesized using fluorenylmethyloxycarbonyl chloride (FMoc)-based solid phase chemistry on Knorr resin, and characterized using mass spectroscopy. Each vWF molecule has only one binding region for this peptide, and hence vascular injury sites presenting multiple vWF binding sites for multiple copies of this peptide decorated on the nanoparticle surface, provide a mechanism for enhanced adhesion of the nanoparticles with increasing shear.

In some embodiments, the CBP can include a peptide that comprises a short seven-repeat of the tripeptide GPO (i.e., [GPO]₇, SEQ ID NO: 2) with a helicogenic affinity to fibrillar collagen. The GPO trimer is based on amino acid repeats found in the native collagen structure. It has been reported that the activation of platelets usually caused by interaction with collagen through GPVI and GPIa/IIa, can also potentially occur when platelets interact with collagen-derived peptides. This can be a potential problem regarding decorating synthetic particle surfaces with collagen-derived peptides for binding of collagen, because in vivo the constructs can potentially interact with quiescent blood platelets and systemically activate them, posing thromboembolic risks. However, interaction of platelet receptors with collagen and the subsequent platelet activation mechanisms are dependent upon receptor clustering induced by multimeric long chain triple-helical fibrillar collagen and not by short collagen-mimetic peptide repeats. In fact, it has been shown that GPO-trimer repeats as high as a 30-mer (10 repeats) only partially interact with platelet GPIa/IIa and GPVI integrins and are incapable of activating platelets; yet they can effectively bind to fibrillar collagen via helicogenic interaction. Hence, this small CBP can promote adhesion to fibrillar collagen, but cannot activate quiescent platelets due to absence of long triple-helical conformation. The CBP like the VBP can also be synthesized using FMoc-based solid phase chemistry on Knorr resin, and characterized using mass spectroscopy.

Advantageously, the VBPs and CBPs can each include about 5 to about 30 amino acids. By limiting the size of the peptides to about 5 to about 30 amino acids, the VBPs and CBPs can be spatially or topographically arranged on the flexible nanoparticle surface such that the VBPs and CBPs do not spatially mask each other and are able to adhere to a vascular surface, vascular disease site, and/or vascular injury site with exposed vWF and collagen and promote arrest and optionally aggregation of active platelets onto sites of the synthetic platelet adhesion.

The ratio of VPBs to CPBs provided on the nanoparticle surface can be about 75:25 to about 25:75 and be adjusted accordingly to maximize adhesion under low-to-high shear conditions.

In other embodiments, the nanoparticles can further include a plurality of fibrinogen mimetic peptides (FMPs) that bind to GPIIb-IIIa, endothelial cell targeting peptides, and/or platelet targeting peptides that are linked to the shell and extend from the outer surface.

In some embodiments, the FMP can include an RGD amino acid sequence motif that promotes active platelet aggregation. The RGD motif containing FMP may contain a single repeat of the RGD motif or may contain multiple repeats of the RGD motif, such as, for example, 2, or 5, or 10 or more repeats of the RGD motif. One of skill in the art will understand that conservative substitutions of particular amino acid residues of the RGD motif containing FMPs may be used so long as the RGD motif containing FMP retains the ability to bind comparably as the native RGD motif. One of skill in the art will also understand that conservative substitutions of particular amino acid residues flanking the RGD motif so long as the RGD motif containing FMP retains the ability to bind comparably as the native RGD motif.

In some embodiments, the FMP can include a cyclic RGD (cRGD) peptide having the amino acid sequence of cyclo-CNPRGDY(OEt)RC (SEQ ID NO: 3). A cyclic peptide having SEQ ID NO: 3 can have high selectivity and affinity to GPIIb-IIIa on activated platelets but do not bind or activate quiescent platelets nor interact with other RGD-binding integrins. The FMP like the VBP and CBP can be synthesized using FMoc-based solid phase chemistry on Knorr resin, and characterized using mass spectroscopy.

The VBPs, CBPs, and optionally, FMPs, endothelial cell targeting peptides, and/or platelet targeting peptides can be conjugated to the nanoparticle surface by reacting the peptides with through their N-termini to the carboxyl termini of a heterobifunctional PEG, such as maleimide-PEG-COOH. The PEG-peptide conjugates or PEGylated peptides can then be conjugated to the nanoparticle using known conjugation techniques.

The PEG molecules can have a variety of lengths and molecular weights, including, for example, PEG 200, PEG 1000, PEG 1500, PEG 4600, PEG 10,000, or combinations thereof. In other embodiments, the VBPs, CBPs, and FMPs can be conjugated to lipids that define the nanoparticle surface with PEG acrylate, PEG diacrylate, or other molecules of a variety of molecular weights.

In some embodiments, the ratio of VPB:CPB:FMP can be about 1:1:2 to 1:2:1 to 2:1:1. It will be appreciated, that other ratios can be used to enhance the nanoparticle adherence and activated platelet aggregation.

In some embodiments, thrombin loaded nanoparticles can provide vascular injury-site targeted delivery of thrombin to a subject. The amount of thrombin delivered to the vascular injury site in the subject by the hemostat is an amount effective to augment hemostasis in the subject.

It will be appreciated that other therapeutic agents or bioactive agents can be encapsulated by, contained in, and/or linked to the nanoparticle. Such therapeutic agents or bioactive agents can include any substance capable of exerting a biological or therapeutic effect in vitro and/or in vivo. Therapeutic agents can also include any therapeutic or prophylactic agent used in the treatment (including the prevention, diagnosis, alleviation, or cure) of a malady, affliction, condition, disease, or injury in a subject. Examples of therapeutic agents include, but are not limited to procoagulants and anti-proliferative agents. The therapeutic agents can be in the form of biologically active ligands, small molecules, peptides, polypeptides, proteins, DNA fragments, DNA plasmids, interfering RNA molecules, such as siRNAs, oligonucleotides, and DNA encoding for shRNA.

In some embodiments, the additional therapeutic agents can be a therapeutic agent suitable for the treatment of blood-associated disorders. Examples of therapeutic agents suitable for the treatment of blood-associated disorders can include at least one of coagulation factor, platelet agonist, antifibrinolytic agents, prothrombin, antiacetaminophen, steroids, hyaluronic acid, glucosamine, chondroitin, shea nut oil extract (shea butter), desmopressin, anti-hemophilic factor recombinant, anti-inhibitor coagulant complex, rituximab, chelation therapy, and nonsteroidal anti-inflammatory drugs (NSAIDs) including COX-2 inhibitors.

Examples of NSAIDs include 2-arylpropionic acids such as ibuprofen, ketorolac and naproxen; n-arylanthranilic acids such as mefenamic acid and meclofenamic acid; oxicams, such as piroxicam and meloxicam; and arylalkanoic acids such as diclofenac, etodolac, indomethacin, and sulindac. Examples of COX-2 inhibitors include celecoxib, etoricoxib, rofecoxib, and valdecoxib. Examples of coagulation factor VIII and factor IX include Helixate, Monoclate-P, Beriate, BeneFix, Alprolix, Idelvion, corticosteroids, and Rixubis.

In some embodiments, the release of thrombin and optional other therapeutic agents from the nanoparticle of the biosynthetic hemostat can in addition to diffusion and enzyme degradation occur by polymer or lipid wall, erosion, and/or disruption of the nanoparticle, which can be controlled by the type of the nanoparticle or microparticle, i.e., having it become swollen or degradable in the chosen microenvironment.

Optionally, the thrombin and optional other therapeutic agent can be released from the nanoparticle through the use of an external trigger, such as light and ultrasound.

Advantageously, a nanoparticle, which allows remote release of the thrombin and optional other therapeutic agents, can target or be targeted to a vascular surface, a vascular disease site and/or a vascular injury site, by systemic parenteral administration (e.g., intravenous, intravascular, or intraarterial infusion) to the subject and once targeted to the site remotely released to specifically treat the vascular disease site and/or vascular injury site tissue of the subject. Targeting and selective release of therapeutic agents allows treatment of such vascular diseases, which would provide an otherwise diminished therapeutic effect if not targeted and remotely released using the compositions described herein. In some embodiments, release of the thrombin and optional other therapeutic agents from the nanoparticle can be triggered by an energy source that supplies energy to the nanoparticles effective to release the thrombin and optional other therapeutic agents from the nanoparticle. The energy source can be external or remote from a subject, which allows non-invasive remote release of the thrombin and optional other therapeutic agents to the subject. The remote energy source can be, for example, a minimally invasive laser that can be inserted in vivo in the subject being treated or positioned external or ex vivo the subject. The energy from laser can be in the near infrared range to allow deep radiation penetration into tissue and remote release of therapeutic agent or imaging agent.

Therefore, in some embodiments, a nanoparticle of the hemostat can be surface modified to be responsive to energy, from a remote source that is effective to release the thrombin and optional other therapeutic agent from the nanoparticle upon mechanical disruption of the nanoparticle membrane or shell after administering the biosynthetic hemostat to a subject.

In an exemplary embodiment, near infra-red (NIR)-responsive gold nanorods (GNRs) conjugated close to the surface of the nanoparticle encapsulating or containing the thrombin and optional other therapeutic agent can exhibit plasmon resonance phenomena under tissue-penetrating NIR light, such that the resultant thermo-mechanical energy dissipation results in disruption of the nanoparticle to render site-selective rapid drug release. Thus, in some embodiments, NIR-irradiation from specialized external or catheter-mediated laser devices can be used to remotely trigger rapid drug release at the targeted disease site via photothermal destabilization of GNR-modified nanoparticles.

It will be appreciated that other remote energy sources can be used to release the therapeutic agent or imaging agent from the nanoparticle or microparticle and that the selection of the energy source will depend at least in part on the nanoparticle used to form the hemostat.

In some embodiments, the nanoparticles described herein can be provided in a pharmaceutical composition. Such a pharmaceutical composition may consist of the nanoparticles alone, in a form suitable for administration to a subject, or the pharmaceutical composition may comprise nanoparticles and one or more pharmaceutically acceptable carriers, one or more additional ingredients, one or more pharmaceutically acceptable therapeutic agents, bioactive agents, diagnostic agents, or some combination of these. The therapeutic agent may be present in the pharmaceutical composition in the form of a physiologically acceptable ester or salt, such as in combination with a physiologically acceptable cation or anion, as is well known in the art.

As used herein, the term “pharmaceutically acceptable carrier” means a chemical composition with which the therapeutic agent may be combined and which, following the combination, can be used to administer the therapeutic agent to a subject.

As used herein, the term “physiologically acceptable” ester or salt means an ester or salt form of the therapeutic agent which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing nanoparticles into association with a carrier or one or more other accessory ingredients, and then, if necessary or desirable, shaping or packaging the product into a desired single- or multi-dose unit.

In still other embodiments, administration of the biosynthetic hemostat can be supplemented or augmented with the administration of exogenous fibrinogen. Fibrinogen (coagulation factor I) is the last protein of the coagulation cascade. It is cleaved by thrombin (factor IIa) to yield a primary unstable fibrin clot, which is further stabilized into firm and stable clot. Fibrinogen is a constant constituent of every blood derived product, and therefore in situations where a replacement therapy with fibrinogen is required these blood derived products are administered to provide fibrinogen. More specifically, Fibrinogen (factor I), as used herein, is a soluble plasma glycoprotein with a molecular weight of approximately 340 kDa and circulates in plasma as a precursor of fibrin. The native molecule is a homo-dimer, in which both subunits consist of three different polypeptide chains (Aα, Bβ, and γ). All three polypeptide chains of the subunits as well as the dimer are linked with disulfide bonds. The three pairs of polypeptide chains named Aα, Bβ, and γ are composed of 610, 461, and 411 amino acids, respectively. Fibrinogen is synthesized in the liver by the hepatocytes. The concentration of fibrinogen in the blood plasma is 200-400 mg/dL (normally measured using the Clauss method).

A variety of Fibrinogen concentrates and products are currently commercially available, to name but a few, Haemocomplettan (CSL Behring, Marburg, Germany), FIBRINOGENE T1 and Clottagen (LFB, Les Ulis, France), Fibrinogen HT (Benesis, Osaka, Japan) and FibroRAAS (Shangai RAAS, Shangai, China). However, the most widely used is Haemocomplettan (commercialized in the USA as RiaSTAP), a human pasteurised, highly purified, plasma-derived fibrinogen concentrate. It should be appreciated that any fibrinogen preparation, for example, any of the preparations disclosed above, may be co-administered with biosynthetic hemostat such that the fibrinogen is isolated from the thrombin prior to administration to the subject.

In some further embodiments the biosynthetic hemostat and optional fibrinogen may be adapted for systemic or parenteral administration to a subjection in need thereof. The phrases “systemic administration”, “administered systemically” as used herein mean the administration of the biosynthetic hemostat and optional fibrinogen directly intravenously into the central blood system, such that it enters the subject's system and, thus, is subject to metabolism and other like processes. The phrases “parenteral administration” and “administered parenterally” as used herein means modes of administration other than enteral and topical administration, usually by injection, and includes, intravenous injection or intra-arterial. In particular, parenteral administration is contemplated to include, but is not limited to, cutaneous, subcutaneous, intraperitoneal, intramuscular, intrasternal injection, intravenous, and intra-arterial.

In yet another embodiment, the biosynthetic hemostat and optional fibrinogen may be adapted for topical administration. By “topical administration” it is meant that the biosynthetic hemostat and optional fibrinogen can be administered locally. Specifically, the biosynthetic hemostat and optional fibrinogen is applied onto a surface by a mean of external injection, spraying or any other superficial application. The biosynthetic hemostat and optional fibrinogen may include any means for local application, or may be designed in a form adapted for local administration, for example, transdermal patches, ointments, lotions, creams, gels, drops, suppositories, sprays, liquids and powders. Still further, it is understood that the term “topical” refers to local application/s that is not a systemic application that although included, is not limited to dermal or transdermal application. Local application may be further applied locally on the treated surface, organ or tissue by using catheters, syringe or any other applicator or any other pouring, dipping, immersing or coating means. Conventional pharmaceutical carriers, aqueous, powder or oily bases, thickeners and the like may be necessary or desirable.

In some embodiments, the biosynthetic hemostat and optional fibrinogen may be adapted for transdermal delivery. Transdermal delivery may be accomplished in various ways. By “transdermal” herein is meant the passing through the skin and into a subject's blood stream, whereby to provide a systemic effect. Whilst the term embraces transmucosal, i.e., passing through mucosal tissue so as to embrace sublingual, buccal, vaginal and rectal delivery, typically transdermal delivery is affected through a subject's skin. For this reason, references are generally made herein to skin for simplicity's sake only although it will be appreciated that the transdermal delivery described herein may also be transmucosal.

As discussed above, the biosynthetic hemostat and optional fibrinogen may include additional components to perform coagulation including the calcium ion, but also antibiotics or growth factors.

In some embodiments, the biosynthetic hemostat and fibrinogen may be provided in kits where the biosynthetic hemostat and fibrinogen are separated prior to administration to the subject. For example, the kit can include a first component that includes the biosynthetic hemostat and a separate second component that includes the fibrinogen. Devices for delivery of the biosynthetic hemostat and fibrinogen can enable reconstitution and mixing of the two components, for example specific double syringes and needles or spray for direct application.

For example, a double-syringe can include a mixer nosecone, topped by a blunt applicator needle, attached to the nozzle to facilitate mixing of the two syringe components, one containing the biosynthetic hemostat and the other fibrinogen. When the common plunger is depressed, the biosynthetic hemostat and the fibrinogen are combined in the nosecone, in equal volumes, to form a sealant that is directly applied to the designated tissues. It should be noted that for topical application of the biosynthetic hemostat and optional fibrinogen any suitable applicator may be used, for example, the applicator described herein above or any modifications thereof.

A pharmaceutical composition described herein may be prepared, packaged, or sold in bulk, as a single unit dose, or as a plurality of single unit doses. As used herein, a “unit dose” is discrete amount of the pharmaceutical composition comprising a predetermined amount of the activity. The amount of the activity is generally equal to the dosage, which would be administered to a subject or a convenient fraction of such a dosage such as, for example, one-half or one-third of such a dosage.

The pharmaceutical composition may be administered to a subject as needed. The pharmaceutical composition may be administered to a subject as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

Other embodiments relate to the use of the biosynthetic hemostat described herein in a method of treating at least one of a trauma-induced coagulopathy, surgery-induced coagulopathy, congenital-induced coagulopathy, and/or drug-induced coagulopathy in subject in need thereof.

In some embodiments, the subject can have or be at increased risk of thrombocytopenia. The thrombocytopenia can be caused by or result from dehydration, leukemia, myelodysplastic syndrome, aplastic anemia, liver failure, sepsis, leptospirosis, congenital amegakaryocytic thrombocytopenia, thrombocytopenia absent radius syndrome, fanconi anemia, Bernard-Soulier syndrome, May-Hegglin anomaly, grey platelet syndrome, Alport syndrome, Wiskott-Aldrich syndrome, idiopathic thrombocytopenia purpura, thrombotic thrombocytopenia purpura, hemolytic-uremic syndrome, disseminated intravascular coagulation, paroxysmal nocturnal hemoglobinuria, antiphospholipid syndrome, systemic lupus erythematosus, post-transfusion purpura, neonatal alloimmune thrombocytopenia, hypersplenism, dengue fever, Gaucher's disease, zika virus, medication-induced thrombocytopenia, niacin toxicity, Lyme disease, and thrombocytapheresis.

In some embodiments, the method may be applicable for the treatment, prevention, prophylaxis, amelioration, inhibition of bleeding, hemostatic disorders and any bleeding or pathologic condition associated therewith in a subject in need thereof. The method can include administering to the subject a therapeutically effective amount of the biosynthetic hemostat and optional fibrinogen or of any composition comprising the same.

In some embodiments, the biosynthetic hemostat and optional fibrinogen administered by the method can be formulated for parenteral administration. Thus, in some embodiments, the biosynthetic hemostat and optional fibrinogen can be administered parenterally to treat the subject.

In certain embodiments, the methods may be particularly applicable for subjects suffering from a hemostatic disorder that may be hereditary or acquired bleeding disorders. Acquired bleeding disorders are disorders where bleeding is induced by an external (acquired) cause, such as trauma, surgery or fibrinolytic treatment. Bleeding disorders caused by inherited deficiencies of one or more coagulation factors are rare disorders distributed worldwide. Homozygotes or compound heterozygotes for the mutant genes responsible for these defects exhibit bleeding manifestations that are of variable severity and usually related to the extent of the decreased activity of the particular coagulation factor.

In some embodiments, the methods are applicable for the treatment, prophylaxis, amelioration, inhibition or delaying the bleeding associated with hereditary hemostatic disorder and undefined bleeding tendency. “Hereditary hemostatic disorder” as used herein relates to a hereditary deficiency in at least one coagulation factor. More specifically, numerous mutations have been identified in genes encoding coagulation factors I, II, V, VII, X and XI, that lead to deficiency of at least one of said factors or to impaired activity thereof. Homozygotes for these mutations exhibit bleeding tendency either spontaneously or following trauma/surgery. Heterozygotes for the various deficiencies rarely display a bleeding tendency. Undefined tendency to bleed, as used herein, relates to a condition of bleeding tendency while a precise diagnosis of this condition cannot be established.

Some patients referred for an evaluation of mild bleeding symptoms have an undiagnosed bleeding tendency that may not have been recognized until a challenging event that induces bleeding, such as surgery or childbirth occur. Clinical variability with regard to bleeding manifestations is common among such individuals, suggesting that environmental and other genetic factors may ameliorate bleeding risks. Although mild bleeding problems may not become evident until exposure to significant hemostatic challenges (such as surgery, dental extractions, major trauma, menarche or childbirth), the predictive risk of bleeding following surgery has not been established for these individuals. Gender has an influence on the manifestations of bleeding. Females are more commonly referred for evaluation because of troublesome bleeding with menses and/or childbirth. In addition, bleeding that persists or becomes problematic 24 hours or longer after dental extractions raises the possibility of a bleeding disorder. Failure to establish a diagnosis in a patient with mild mucocutaneous bleeding is a common problem in practice.

Normal laboratory tests are a hallmark for diagnosis of the undefined bleeding tendency. Failure to establish the diagnosis can be problematic for patient who needs to undergo surgery or childbirth.

For mild bleeding symptoms of patients with undefined bleeding disorders, the biosynthetic hemostat and optional fibrinogen may be used for dental and oral surgeries and it can reduce bleeding with other operative procedures. In case severe bleeding develops, for example, during surgery or childbirth the biosynthetic hemostat and optional fibrinogen may be required.

It should be appreciated that the biosynthetic hemostat and optional fibrinogen and compositions described herein, and methods of their use, may be applicable for any form of bleeding that accompanies hereditary hemostatic disorders caused by a deficiency in at least one of factor XI, factor X, factor V, factor VII, factor II (prothrombin) and factor I (fibrinogen) as disclosed herein.

In further embodiments, the methods described herein may be applicable for treating disorders characterized by hereditary deficiencies of the coagulation factors I, II, V, VII, X and XI that include at least one of or any bleeding tendency associated therewith. Hereditary deficiencies of the coagulation factors I, II, V, VII, X and XI are autosomal recessive bleeding disorders that have been described in most populations. Their relative frequency varies among populations partly as a result of high frequencies of specific mutant genes in inbred populations. Several population surveys indicate that common among these bleeding disorders are factors XI and VII deficiency, less common disorders are factors V and X deficiency and afibrinogenemia, and the rarest disorders are factor II (prothrombin) and factor XIII deficiency. The severity of bleeding manifestations in affected patients who are homozygotes or compound heterozygotes for a mutant gene is variable and usually related to the extent of the deficiency. Some patients have only mild bruising or display excessive bleeding only following trauma. Other patients, usually with less than 1 percent of normal factor VII, XIII, or X activity, can exhibit intracranial hemorrhages and hemarthroses similar to patients with severe hemophilia.

In some specific embodiments, the biosynthetic hemostat and optional fibrinogen can be used for treating, preventing, reducing attenuating or inhibiting bleeding associated with hereditary factor XI deficiency, or any acquired bleeding or hemostatic condition in patients suffering from factor XI deficiency. Hereditary factor XI deficiency is transmitted as autosomal recessive trait. The disorder is exhibited in homozygotes or compound heterozygotes as a mild to moderate bleeding tendency that is mainly injury related. Affected subjects have been described in most populations but in Jews, particularly of Ashkenazi origin, the disorder is common. Factor XI deficiency as a result of a dysfunctional protein is rare and the majority of the patients have a decreased Factor XI protein level. Altogether, greater than 150 mutations have been reported in non-Jewish and Jewish patients of various origins, most of them being missense mutations.

Most bleeding manifestations in homozygotes and compound heterozygotes are injury related. Excessive bleeding can occur at the time of injury or begin several hours or days following trauma. The bleeding tendency varies depending upon the hemostatic challenge and the variable sites of injury. Surgical procedures involving tissues with high fibrinolytic activity (urinary tract, tonsils, nose, tooth sockets) frequently are associated with excessive bleeding in patients with severe factor XI deficiency, irrespective of the genotype. Factor XI deficiency by itself is associated with increased fibrinolysis, therefore, the additional bleeding risk of surgery at sites rich in fibrinolysis in these patients may increase the bleeding tendency even further.

In other embodiments, the biosynthetic hemostat and optional fibrinogen can be used for treating, preventing, reducing attenuating, inhibiting bleeding associated with hereditary factor VII deficiency, or any acquired bleeding or hemostatic condition in patients suffering from factor VII deficiency. Hereditary deficiency of factor VII is a rare autosomal recessive disorder that has been observed in most populations. A presumptive diagnosis can be easily made because factor VII deficiency is the only coagulation disorder that produces a prolonged clotting time test prothrombin time (PT). Most mutations causing factor VII deficiency have been missense mutations.

Bleeding manifestations occur in homozygotes and in compound heterozygotes for factor VII deficiency. Patients who have factor VII activity less than 1 percent of normal, frequently present a severe bleeding manifestations such as hemarthroses leading to severe arthropathy and life-threatening intracerebral hemorrhage. Patients with slightly higher levels of factor VII (factor VII activity of 5 percent of normal or more) have a much milder disease, characterized by epistaxis, gingival bleeding, menorrhagia, and easy bruising. Some surgical procedures such as dental extractions, tonsillectomy, and procedures involving the urogenital tracts frequently are accompanied by bleeding when no prior therapy is instituted prior to the procedure. In contrast, surgical procedures such as laparotomy, herniorrhaphy, appendectomy, and hysterectomy have been uneventful.

In yet further embodiments, the biosynthetic hemostat and optional fibrinogen can be used for treating, preventing, reducing attenuating, inhibiting bleeding associated with hereditary factor X deficiency, or any acquired bleeding or hemostatic condition in patients suffering from factor X deficiency. Hereditary factor X deficiency, a moderate to severe bleeding tendency, is an autosomal recessive disorder. The currently described 95 mutations that cause factor X deficiency include large deletions, small frameshift deletions, nonsense mutation, and missense mutations. The clinical manifestations of factor X deficiency are related to the functional levels of factor X. Individuals with severe factor X deficiency and functional factor X levels less than 1 percent of normal bleed spontaneously and following trauma. Bleeding occurs primarily into joints and soft tissues, however, bleeding from mucous membranes such as Menorrhagia may be especially problematic in women. More unusual bleedings are intracerebral hemorrhage, intramural intestinal bleeding (which can produce symptoms like those of an acute abdomen), urinary tract bleeding, and soft tissue bleeding with development of hemorrhagic pseudocysts or pseudotumors. In individuals with mild deficiencies of factor X bleeding is less common, usually occurring only after trauma or during or after surgery.

In yet another embodiment, the biosynthetic hemostat and optional fibrinogen can be used for treating, preventing, reducing attenuating, and inhibiting bleeding associated with hereditary factor V deficiency, or any acquired bleeding or hemostatic condition in patients suffering from factor V deficiency. Hereditary factor V deficiency is among the less common inherited bleeding disorders and manifests in homozygotes or compound heterozygotes as a moderate bleeding tendency. Factor V deficiency is inherited as an autosomal recessive trait. Heterozygotes, whose plasma factor V activity ranges between 25 and 60 percent of normal, usually are asymptomatic, Assays of factor V protein indicate that most homozygotes and compound heterozygotes have a true deficiency rather than a dysfunctional protein. Above 80 total distinct mutations have been identified, of which one quarter are missense, Homozygous or compound heterozygous patients whose factor V level ranges from less than 1 to 10 percent of normal exhibit a lifelong bleeding tendency. Common manifestations include ecchymoses, epistaxis, gingival bleeding, hemorrhage following minor lacerations, and menorrhagia. Postpartum hemorrhage occurs in more than 50 percent of pregnancies in patients with severe factor V deficiency. Bleeding from other sites is less common. Trauma, dental extractions, and surgery confer a high risk of excessive bleeding.

In other embodiments, the biosynthetic hemostat and optional fibrinogen can be used for treating, preventing, reducing attenuating, inhibiting bleeding associated with hereditary factor II deficiency, or any acquired bleeding or hemostatic condition in patients suffering from factor II deficiency. Inherited factor II (prothrombin) deficiency is one of the rarest coagulation factor deficiencies. It presents in two forms: type I, true deficiency (hypoprothrombinemia), and type II, in which dysfunctional prothrombin is produced (dysprothrombinemia). These autosomal recessive disorders are genetically heterogeneous, and characterized by a mild to moderate bleeding tendency.

Abnormalities of prothrombin are inherited in an autosomal recessive manner. Among individuals with type I deficiency, heterozygotes exhibit prothrombin levels that are approximately 50 percent of normal, whereas homozygotes display levels that typically are less than 10 percent of normal. Above fifty mutations that cause prothrombin deficiency have been identified, most of which are missense mutations.

Inherited types I and II deficiencies are characterized by mild to moderate mucocutaneous and soft-tissue bleeding that usually correlates with the degree of functional prothrombin deficiency. With prothrombin levels of approximately 1 percent of normal, bleeding may occur spontaneously or following trauma. Surgical bleeding may be significant. Menorrhagia, epistaxis, gingival bleeding, easy bruising, and subcutaneous hematomas may occur.

In yet another embodiment, the biosynthetic hemostat and optional fibrinogen can be used for treating, preventing, reducing attenuating, inhibiting bleeding associated with hereditary fibrinogen deficiency or any acquired bleeding or hemostatic condition in patients suffering from hereditary fibrinogen deficiency. “Fibrinogen (factor I) deficiency” as used herein relates to hereditary fibrinogen abnormalities comprises the afibrinogenemia (complete absence of the fibrinogen), dysfibrinogenemia and hypodysfibrinogenemia. Inherited disorders of fibrinogen are rare and can be subdivided into type I and type II disorders. Type I disorders (afibrinogenemia and hypofibrinogenemia) affect the quantity of fibrinogen in circulation. Type II disorders (dysfibrinogenemia and hypodysfibrinogenemia) affect the quality of circulating fibrinogen. Afibrinogenemia, the most severe form of fibrinogen deficiency, is characterized by autosomal recessive inheritance and the complete absence of fibrinogen in plasma.

Dysfibrinogenemia is defined by the presence of normal levels of functionally abnormal plasma fibrinogen. Hypodysfibrinogenemia is defined by low levels of a dysfunctional protein. These are heterogeneous disorders caused by many different mutations in the three fibrinogen coding genes. Dysfibrinogenemias and hypodysfibrinogenemias are autosomal dominant disorders. Most affected patients are heterozygous for mis sense mutations in the coding region of one of the three fibrinogen genes. Because the secreted fibrinogen hexamer contains two copies of each of the three fibrinogen chains, and the resulting fibrin network contains multiple copies of the molecule, heterozygosity for one mutant allele is sufficient to impair the structure and function of the fibrin clot.

Bleeding because of a fibrinogenemia usually manifests in the neonatal period, with 85 percent of cases presenting umbilical cord bleeding, but a later age of onset is not unusual. Bleeding may occur in the skin, gastrointestinal tract, genitourinary tract, or the central nervous system with intracranial hemorrhage being the major cause of death. There is an intriguing susceptibility of spontaneous rupture of the spleen in afibrinogenemic patients. Menstruating women may experience menometrorrhagia. In addition, first trimester abortion is usual in afibrinogenemic women. These patients may also have antepartum and postpartum hemorrhage. Hemoperitoneum after rupture of the corpus luteum has also been observed.

In some specific embodiments, the biosynthetic hemostat and optional fibrinogen can be used for treating, preventing, reducing, attenuating, and inhibiting bleeding associated with surgical procedures, specifically, minor or major surgical procedures. Surgical procedures are a challenge to the hemostatic system, especially when surgery is performed at places rich in fibrinolytic proteins. Even patients with no or mild to moderate bleeding disorders can bleed excessively following surgery. In addition to the extent of the surgical trauma, the magnitude of the fibrinolytic activity at the surgical site must be considered.

In other embodiments, the biosynthetic hemostat and optional fibrinogen described herein can be used to treat a bleeding disorder. In one embodiment, the bleeding disorder is hemophilia. In a further embodiment, the hemophilia is hemophilia A. In yet another embodiment the hemophilia is hemophilia B. In one embodiment, the hemophilia is hemophilia A. In another embodiment, the hemophilia is acquired hemophilia A with inhibitory auto antibodies to FVIII. In one embodiment, the hemophilia is congenital hemophilia B with inhibitors. In another embodiment, the hemophilia is acquired hemophilia B with inhibitory auto antibodies to FIX.

In other embodiments, the bleeding disorder is a non-hemophilia bleeding disorder. In one embodiment, the bleeding disorder is blood loss from trauma. In another embodiment, the bleeding disorder is FVII deficiency. In one embodiment, the bleeding disorder is FV deficiency. In another embodiment, the bleeding disorder is FX deficiency. In one embodiment, the bleeding disorder is FXI deficiency. In one embodiment, the bleeding disorder is FXIII deficiency. In one embodiment, the bleeding disorder is fibrinogen deficiency. In one embodiment, the bleeding disorder is prothrombin deficiency. In another embodiment, the bleeding disorder is dilutional coagulopathy. In a further embodiment, the bleeding disorder is thrombocytopenia. In yet another embodiment, the bleeding disorder is blood loss from high-risk surgeries. In another embodiment, the bleeding disorder is intracerebral hemorrhage. In one embodiment, the bleeding disorder is von Willebrand disease. In a further embodiment, the bleeding disorder is von Willebrand disease with inhibitors to von Willebrand factor.

In other embodiments, the bleeding disorder is a congenital platelet function defect, including, but not limited to, platelet storage pool disorder, Glanzmann's thrombasthenia, or Bernard-Soulier syndrome. In one embodiment, the bleeding disorder is an acquired platelet function defect. In one embodiment, the bleeding disorder is a congenital deficiency of Factor II, Factor V, Factor VII, Factor X, or Factor XI. In one embodiment, the bleeding disorder is neonatal and pediatric coagulopathies. In one embodiment, the bleeding disorder is a platelet function disorder. In another embodiment, the bleeding disorder is heparin-induced thrombocytopenia. In one embodiment, the bleeding disorder is disseminated intravascular coagulation.

In other embodiments, the non-hemophilia bleeding disorder is blood loss from trauma. In another embodiment, the non-hemophilia bleeding disorder is FVII deficiency. In one embodiment, the non-hemophilia bleeding disorder is FV deficiency. In another embodiment, the non-hemophilia bleeding disorder is FX deficiency. In one embodiment, the non-hemophilia bleeding disorder is FXI deficiency. In one embodiment, the non-hemophilia bleeding disorder is FXIII deficiency. In one embodiment, the non-hemophilia bleeding disorder is fibrinogen deficiency. In one embodiment, the non-hemophilia bleeding disorder is prothrombin deficiency. In another embodiment, the non-hemophilia bleeding disorder is dilutional coagulopathy. In a further embodiment, the non-hemophilia bleeding disorder is thrombocytopenia. In yet another embodiment, the non-hemophilia bleeding disorder is blood loss from high-risk surgeries. In another embodiment, the non-hemophilia bleeding disorder is intracerebral hemorrhage. In one embodiment, the non-hemophilia bleeding disorder is von Willebrand disease. In a further embodiment, the non-hemophilia bleeding disorder is von Willebrand disease with inhibitors to von Willebrand factor.

In one embodiment, the non-hemophilia bleeding disorder is a congenital platelet function defect, including, but not limited to, platelet storage pool disorder, Glanzmann's thrombasthenia, or Bernard-Soulier syndrome. In one embodiment, the non-hemophilia bleeding disorder is an acquired platelet function defect. In one embodiment, the non-hemophilia bleeding disorder is a congenital deficiency of Factor II, Factor V, Factor VII, Factor X, or Factor XI. In one embodiment, the non-hemophilia bleeding disorder is neonatal and pediatric coagulopathies. In one embodiment, the non-hemophilia bleeding disorder is a platelet function disorder. In another embodiment, the non-hemophilia bleeding disorder is heparin-induced thrombocytopenia. In one embodiment, the non-hemophilia bleeding disorder is disseminated intravascular coagulation. In other embodiments, the non-hemophilia bleeding disorder is any disorder known to one of skill in the art.

In some embodiments, the biosynthetic hemostat and optional fibrinogen can be used for treating acquired hemostatic disorders. The acquired hemostatic disorder may be at least one of surgery-induced bleeding, trauma-induced bleeding, acute gastrointestinal bleeding, bleeding associated with burns, hemorrhagic stroke, lung injury associated with emphysema and chronic obstructive pulmonary disease (COPD), bleeding associated with childbirth, disseminated intravascular coagulation (DIC), and bleeding resulting from fibrinolytic or thrombolytic therapy.

It should be understood that in cases where the surgical procedures are elective, expected or not urgent (e.g., cesarean surgery, or any other major surgery that allow sufficient time for pre-operative preparations), the biosynthetic hemostat and optional fibrinogen can be used for pre-operative treatment to facilitate prevention or reduction of excessive bleeding during the surgical intervention. Thus, in some embodiments, the biosynthetic hemostat and optional fibrinogen may provide a preventive method particularly useful for patients having hereditary disorders, patients suffering from hyperfibrinolysis and/or patients that are expected to be operated.

In a further embodiment, the biosynthetic hemostat and optional fibrinogen can be used for treating trauma-induced bleeding (traumatic bleeding). Traumatic bleeding can be caused by any type of injury, for example any injury caused by, work and car accidents, combats or falls. There are different types of traumatic wounds which may cause bleeding. In general, trauma causes damage to a blood vessels that in turn causes blood to flow externally outside the body or internally into body organs, such as brain, lung, liver, kidney, spleen or into body cavities, such as thorax and abdomen.

Beside the physical measures to stop the bleeding, the biosynthetic hemostat and optional fibrinogen can be administered to initiate blood clotting, which will eventually result in a cessation of bleeding.

In other embodiments, the biosynthetic hemostat and optional fibrinogen can be used for treating acute or chronic gastrointestinal bleeding. “Gastrointestinal (GI) bleeding”, also known as gastrointestinal hemorrhage, as used herein, relates to all forms of bleeding in the gastrointestinal tract, from the mouth to the rectum. “Acute gastrointestinal bleeding” means that there is a significant blood loss over a short time causing acute blood loss and hemorrhagic shock. Symptoms may include vomiting (hemathemesis) either red blood or black blood (due to digested blood also called “coffee ground”), bloody stool, or black stool (digested blood called melena). In contrast, chronic gastrointestinal bleeding is bleeding of small amounts of blood over a long time. In this case the symptoms are of iron-deficiency anemia.

GI bleeding is typically divided into two main types: upper gastrointestinal bleeding and lower gastrointestinal bleeding. Causes of upper GI bleeds include: peptic ulcer disease, esophageal varices, that may occur in some embodiments, due to liver cirrhosis and cancer, among others. Causes of lower GI bleeds include: hemorrhoids, cancer, and inflammatory bowel disease among others. Endoscopy of the lower and upper gastrointestinal track may locate the area of bleeding. Medical imaging may be useful in cases that are not clear.

Lower gastrointestinal bleeding is typically from the colon, rectum or anus. Common causes of lower gastrointestinal bleeding include hemorrhoids, cancer, angiodysplasia, ulcerative colitis, Crohn's disease, and aortoenteric fistula.

In other embodiments, the biosynthetic hemostat and optional fibrinogen can be used for treating burns, and specifically, bleeding associated with burns. A burn is a type of injury to skin, or other tissues, caused by heat, cold, electricity, chemicals, friction, or radiation. Most burns are due to heat from hot liquids, solids, or fire. In large burns (over 30% of the total body surface area), there is a significant inflammatory response. This results in increased leakage of fluid from the capillaries, and subsequent tissue edema. This causes overall blood volume loss, with the remaining blood suffering significant plasma loss, making the blood more concentrated. Poor blood flow to organs such as the kidneys and gastrointestinal tract may result in renal failure and stomach ulcers. Blood transfusions when required are recommended when the hemoglobin level falls below 6-8 g/dL. Plasma is administered as a colloid volume expander fluid. Thus, when there is an indication to use blood or plasma and related products for the treatment of burns in a subject in need thereof, the biosynthetic hemostat and optional fibrinogen be used for treating subjects affected by any burn.

In yet further embodiments, the biosynthetic hemostat and optional fibrinogen can be used for treating hemorrhagic stroke or any other brain injury or trauma. “Hemorrhagic stroke” as used herein, relates to bleeding occurring directly into the brain parenchyma. The usual mechanism is thought to be leakage from small intracerebral arteries damaged by chronic hypertension. Patients with intracerebral bleeds are more likely than those with ischemic stroke to have headache, altered mental status, seizures, nausea and vomiting, and/or marked hypertension. Even so, none of these findings reliably distinguishes between hemorrhagic and ischemic stroke.

In other embodiments, the biosynthetic hemostat and optional fibrinogen can be used for treating, preventing, reducing, attenuating, and inhibiting bleeding associated with major surgery. Major surgery is defined as any surgical procedure that involves anesthesia or respiratory assistance.

In particular embodiments, the biosynthetic hemostat and optional fibrinogen can be used during open heart surgery. Some surgical procedures can be anticipated to cause severe bleeding, such as open heart surgery. In these operations extracorporeal circulation (cardiopulmonary bypass—CPB) is used. Cardiovascular (open heart) surgery is surgery on the heart or great vessels performed by cardiac surgeons. Frequently, it is done to treat complications of ischemic heart disease (for example, coronary artery bypass grafting), correct congenital heart disease, or treat valvular heart disease from various causes including endocarditis, rheumatic heart disease and atherosclerosis. It also includes heart transplantation. During open-heart surgery, the heart is temporarily stopped. Patients undergoing an open-heart surgery are placed on cardiopulmonary bypass, meaning a machine which pumps their blood and oxygen for them.

The bleeding phenomena that occur in these operations are due to the anticoagulation used during the surgery, which, deliberately induces coagulation deficiency. In addition, platelet dysfunction that stems from the passing of the blood through an extracorporeal circulation contributes to the tendency to bleed.

In further embodiments, the biosynthetic hemostat and optional fibrinogen can be used to treat bleeding associated with liver transplantation surgery. The liver plays a central role in hemostasis and thrombosis. Liver parenchymal cells are the site of synthesis of most coagulation factors, the physiologic inhibitors of coagulation, and essential components of the fibrinolytic system. The liver also regulates hemostasis and fibrinolysis by clearing activated coagulation factors and enzyme inhibitor complexes from the circulation. Therefore, when liver dysfunction occurs in patients with liver disease, a complicated hemostatic derangement ensues, which can lead to bleeding.

During the first stage of liver transplantation, the removal of the diseased liver, (the anhepatic stage), significant hemostatic changes can occur. Because activated clotting factors are not removed from the circulation, their consumption can develop together with consumption of platelets and secondary hyperfibrinolysis. The most severe hemostatic changes during liver transplantation occur after reperfusion of the donor liver. Platelets are trapped in the graft, giving rise to an aggravation of thrombocytopenia and causing damage to the graft by induction of endothelial cell apoptosis. Thus, hyperfibrinolysis is thought to contribute significantly to impaired hemostasis during the hepatic and reperfusion phases. Moreover, the graft releases heparin-like substances that can inhibit coagulation. In addition, other factors such as hypothermia, metabolic acidosis, and hemodilution adversely affect hemostasis during this phase. Liver transplantation is a lengthy procedure with extensive surgical wound surfaces including potential transaction of collateral veins. Improved surgical techniques and anesthesiologic care have led to a remarkable reduction of blood loss during liver transplantation. Thus, it should be appreciated that the biosynthetic hemostat and optional fibrinogen may be applicable for cessation of bleeding associated with hyperfibrinolytic state induced by liver transplantation surgery.

It should be appreciated that the biosynthetic hemostat and optional fibrinogen can be used for any surgery involving any organ or tissue transplantation, for example, liver, kidney, lung, heart, pancreas, skin, blood vessels and the like.

In yet another embodiment, the biosynthetic hemostat and optional fibrinogen can be used for treating bleeding induced by fibrinolytic/thrombolytic therapy. Fibrinolytic/thrombolytic therapy is mostly administered in patients with acute myocardial infarction (acute coronary artery thrombosis) or in patients with acute stroke (acute cerebral arterial thrombosis). The goal of fibrinolytic/thrombolytic therapy is rapid restoration of blood flow in an occluded vessel achieved by accelerating fibrinolytic proteolysis of the thrombus. Fibrinolytic therapy typically results in fibrinolytic state because plasminogen activation is not limited to the thrombus. These effects are complex and include a reduction in fibrinogen level, increase in fibrinogen degradation products, and decreases in coagulation factors. The complication of fibrinolytic therapy is bleeding. Bleeding complications are more frequent with fibrinolytic than with anticoagulant therapy and require rapid diagnosis and management. Two problems contribute to excess bleeding. First, the fibrinolytic effect is not limited to the site of thrombosis but is usually systemic. Therefore, any hemostatic plugs needed to prevent bleeding at sites of vascular injury caused either by catheters needed for treatment or within pathologic lesions in the brain, gastrointestinal tract, or elsewhere are also susceptible to dissolution. The most serious complication is intracranial hemorrhage which occurs in approximately 1% of patients and is associated with a high mortality and serious disability in survivors. The most common bleeding complications are related to invasive vascular procedures such as placement of arterial and venous catheters. Some bleeding at these sites is frequent and should not be a reason for interrupting therapy if it can be managed with local pressure or other simple measures. The problem can be minimized by limiting venous and arterial punctures and by early institution of local measures. Major bleeding may also result from preexisting lesions such as gastrointestinal ulcers or genitourinary lesions.

In further embodiments, the biosynthetic hemostat and optional fibrinogen can be used for treating, prevention, prophylaxis amelioration, inhibition of any bleeding associated with childbirth or pregnancies, for example, postpartum hemorrhage (PPH). Postpartum bleeding or postpartum hemorrhage (PPH) is often defined as the loss of more than 500 ml or 1,000 ml of blood within the first 24 hours following childbirth. Signs and symptoms may initially include: an increased heart rate, feeling faint upon standing, and an increased breath rate. The condition can occur up to six weeks following delivery. The most common cause is poor contraction of the uterus following childbirth, the fact that not all of the placenta was delivered, a tear of the uterus, or poor blood clotting.

In some embodiments, the biosynthetic hemostat and optional fibrinogen can be used for treating Goodpasture syndrome (GPS). GPS is a rare autoimmune disease in which antibodies attack the basement membrane in lungs and kidneys, leading to bleeding from the lungs and kidney failure.

In further embodiments, the biosynthetic hemostat and optional fibrinogen can be used for treating bleeding caused by vessel rupture.

Still further, as noted herein above, the biosynthetic hemostat and optional fibrinogen can be used for treating, prevention, prophylaxis amelioration, inhibition of any bleeding tendency using an extracorporeal apparatus.

In a specific embodiment the extracorporeal apparatus is a pheresis apparatus or a cardio-pulmonary bypass (CPB).

In other embodiments, the biosynthetic hemostat and optional fibrinogen can be used for treatment, prevention, prophylaxis, amelioration, and/or inhibition of bleeding, hemostatic disorders and any bleeding or pathologic condition where topical administration of the biosynthetic hemostat and optional fibrinogen is needed. In some further embodiments, the biosynthetic hemostat and optional fibrinogen may be suitable for the topical use as a biological glue/sealant for the treatment, prophylaxis, amelioration, inhibition or delaying the onset of bleeding induced by a major or minor surgical operation. In contrast to major surgery that, as detailed above herein, relates to any surgical procedure that involves anesthesia or respiratory assistance, minor surgery is a medical procedure involving an incision with instruments, performed to repair damage or arrest disease in a living body. Since minor surgery includes an incision or cutting, which is an act of penetrating or opening with a sharp edge of any part of a human body, in a subject with bleeding tendency this procedure may induce significant bleeding. Thus, the biosynthetic hemostat and optional fibrinogen are particularly applicable for topical use as biological glue/sealant in the treatment, prophylaxis, amelioration, inhibition or delaying the onset of bleeding induced by major and minor surgical procedures.

As noted above, the biosynthetic hemostat and optional fibrinogen can be used in methods for treating disorders as specified above. The term “treatment” as used herein refers to the administering of a therapeutic amount of the biosynthetic hemostat and optional fibrinogen which is effective to ameliorate undesired symptoms associated with a disease, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease from occurring or a combination of two or more of the above. The treatment may be undertaken when a hemostatic condition initially develops, or may be a continuous administration, for example by administration more than once per day, every 1 day to 7 days, every 7 day to 15 days, every 15 day to 30 days, every month to two months, every two months to 6 months, or even more, to achieve the above-listed therapeutic effects.

In some embodiments, the biosynthetic hemostat and optional fibrinogen can be administered to the subject at an amount effective to provide targeted thrombosis at the site of vascular injury but not cause systemic thrombosis in the subject.

In therapeutic application, the biosynthetic hemostat and optional fibrinogen can be administered to a subject already affected by a bleeding disorder or will manifest with bleeding symptoms in different situations that induce bleeding, specifically, in an amount sufficient to cure or at least partially arrest the bleeding and its complications without causing systemic thrombosis. An amount adequate to accomplish this is defined as a “therapeutically effective dose.” Amounts effective for this use will depend upon the severity of the condition and the general state of the patient.

Single or multiple administrations on a daily, weekly or monthly schedule can be carried out with dose levels and pattern being selected by the treating physician. More specific embodiments relate to the use of typically 2-3 doses per week.

Still further, the biosynthetic hemostat and optional fibrinogen products, composition/s and kit/s and any components thereof may be applied as a single daily dose or multiple daily doses, preferably, every 1 to 7 days. It is specifically contemplated that such application may be carried out once, twice, thrice, four times, five times or six times daily, or may be performed once daily, once every 2 days, once every 3 days, once every 4 days, once every 5 days, once every 6 days, once every week, two weeks, three weeks, four weeks or even a month. The application of the biosynthetic hemostat and optional fibrinogen products, composition/s and kit/s or of any component thereof may last up to a day, two days, three days, four days, five days, six days, a week, two weeks, three weeks, four weeks, a month, two months three months or even more. Specifically, application may last from one day to one month. Most specifically, application may last from one day to 7 days.

It should be appreciated that the methods are not limited to any rout of administration. Specifically, the biosynthetic hemostat and optional fibrinogen products, composition/s and kit/s may be administered either systemically, or locally, for example, topically.

In yet a further aspect, the biosynthetic hemostat and optional fibrinogen can be provided in a kit. In some embodiments, the kit may comprise the biosynthetic hemostat and optionally fibrinogen and at least one coagulation promoting agent. In some specific embodiments, the kit may further comprise calcium.

Optionally, each of the biosynthetic hemostat and fibrinogen may be provided in separate compartments. This may facilitate the treatment of diseases and conditions with a combination of active ingredients that may be kept and optionally administered separately. The kit may further provide a convenient modular format of the different constituents of the compounds and related components required for treatment and allows the required flexibility in therapeutic procedures.

In some embodiments, the kit may further include a container means for containing separate products, such as a divided bottle or a divided foil packet. However, the separate products may also be contained within a single, undivided container. Typically, the kit includes directions for the administration of the separate components. As noted above, the kit form may be particularly advantageous when the separate components are preferably administered in different dosage forms (e.g., parenteral vs. topical), are administered at different dosage intervals, or when titration of the individual components of the combination is desired by the prescribing physician.

According to some embodiments, the kit described herein is intended for achieving a therapeutic effect in a subject suffering from disorders associated with bleeding. Achieving a therapeutic effect is meant for example, where the kit is intended for the treatment of a specific disorder, such as bleeding or hereditary or acquired pathologic condition associated therewith in subject in need thereof. It should be further noted that the application of the kit or any component thereof, may form a complementary treatment regimen for subjects suffering from any of the pathological disorders or diseases as discussed above.

The following example is for the purpose of illustration only and is not intended to limit the scope of the claims, which are appended hereto.

### EXAMPLE

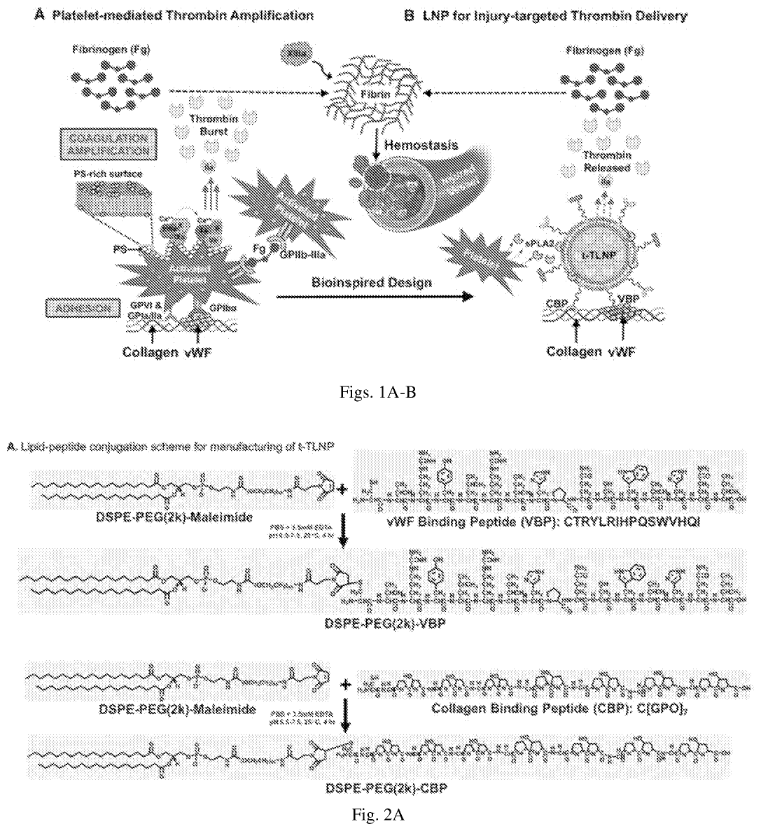

This example describes the development and use of an intravenously or topically administered, injury-site-targeted, enzyme-responsive direct delivery of thrombin using platelet-inspired nanoparticles to site-specifically augment hemostasis. Our design inspiration stems from platelets' critical mechanisms of hemostasis by (i) rapidly adhering and aggregating at the injury site to form a plug (primary hemostasis) and (ii) serving as a coagulation amplifier via presenting an anionic phospholipid such as phosphatidylserine (PS) on the activated platelet surface to render coagulation factor assemblies forming tenase and prothrombinase complexes leading to thrombin (FIIa) burst, which then locally converts fibrinogen (Fg) to fibrin for hemostatic action (FIG. 1A). Without platelets, only a modest amount of thrombin is created by the tissue factor (TF)-FVIIa pathway, which is responsible for initiation of the hemostatic process but not sufficient to rapidly amplify fibrin formation for a stable clot. This is possibly why clinical studies have indicated major survival benefits of early platelet transfusion in trauma. However, there are tremendous challenges for platelet usage, due to the limited donor system that can specifically accumulate at the injury site and directly deliver thrombin can site-specifically augment fibrin independent of native platelet and coagulation status or therapeutic availability of blood products. Thrombin delivery to augment hemostasis is clinically well accepted, as exemplified by products such as Tisseel, Surgiflo, Floseal, etc., where thrombin is delivered via a syringe device directly at the wound site to form fibrin in situ. Research has also been dedicated toward loading thrombin into hemostatic bandages/dressings, as well as topically administering thrombin-loaded particles to mitigate bleeding. However, these systems are only suitable for external administration scenarios and cannot be used intravenously.

Our approach efficiently addresses these challenges by (i) loading a consistent amount of thrombin in lipid nanoparticles (LNPs), (ii) directly targeting LNPs to the injury site via specific binding to vWF and collagen, and (iii) releasing the thrombin with reproducible kinetics triggered by the injury-site-specific enzyme secretory phospholipase A₂ (sPLA₂) for site-localized fibrin production (FIG. 1B). We describe herein the evaluation of these injury-site-targeted thrombin-loaded LNPs (t-TLNPs) in vitro in human blood and plasma, where hemostatic defects were created by platelet depletion and anticoagulant treatment. Spectrophotometric studies of fibrin generation, rotational thromboelastometry (ROTEM) based studies of clot characteristics, and BioFlux microfluidics based real-time imaging of fibrin generation under simulated vascular flow confirmed the ability of t-TLNPs to restore fibrin in hemostatic dysfunction settings. Finally, the in vivo feasibility of t-TLNPs was tested in a mouse tail-bleed model, where a combination of antibody-induced thrombocytopenia plus heparin-induced anticoagulation was used to render significant hemostatic defects and treatment with t-TLNPs was able to effectively reduce bleeding. The t-TLNPs were also evaluated in a traumatic liver injury bleeding model in anticoagulated (with heparin) mice and here treatment with t-TLNPs post-injury significantly reduced bleeding while maintaining systemic safety.

### Methods

### Study Design

The purpose of the study was to test the hypothesis that nanoparticles which mimic injury-site-targeted specific adhesion mechanisms of platelets via binding to vWF and collagen, and mimic the procoagulant thrombin amplification function of platelets by the site-localized direct delivery of thrombin, can locally generate fibrin from fibrinogen to augment hemostasis even when the native platelets are depleted and endogenous coagulation reactions are inhibited. Such an intravenous nanomedicine system enabling targeted direct delivery of thrombin can provide significant benefit in treating bleeding complications stemming from congenital, drug-related, disease-associated, and trauma-induced platelet and coagulation dysfunctions. To achieve platelet-mimetic vWF binding and collagen binding, small-molecular-weight peptides having vWF and collagen adhesion specificities were synthesized and decorated on a liposomal nanoparticle surface. Liposomes were used as a model nanoparticle platform here because of their established clinical history and their capability to enable combinatorial peptide decorations via lipid-peptide self-assembly. The resultant “VBP+CBP”-decorated liposomes were loaded with thrombin. DSPC was used as one of the lipid components of these liposomal systems, since DSPC is amenable to degradation by injury-site-specific enzyme sPLA2, resulting in the destabilization of the injury-site-anchored liposomes for enhanced release of thrombin that can then generate fibrin locally at a rapid rate for hemostatic action. These targeted thrombin-loaded lipid nanoparticles (t-TLNPs) were characterized for their size using dynamic light scattering (DLS) and cryo-transmission electron microscopy (Cryo-TEM). The thrombin loading and release kinetics from t-TLNPs were characterized by spectrophotometric assays using an appropriate thrombin ELISA. The ability of t-TLNPs to undergo platelet-mimetic adhesion on an injury-site-relevant “vWF+collagen”-coated surface under flow was confirmed using BioFlux microfluidics imaged with inverted fluorescence microscopy. The physiologically relevant safety parameters of t-TLNPs were assessed by evaluating the t-TLNP incubation effect on healthy endothelial monolayer activation, resting platelet activation/aggregation, neutrophil activation/NET-osis, and plasma complement factor C3 activation. The ability of t-TLNPs (without and with sPLA₂ trigger) to rescue fibrin generation in anticoagulant-treated and platelet-depleted human plasma was established using an optical density (OD) based method to record fibrin kinetics. In these studies, normal plasma (no platelet depletion or anticoagulation-associated hemostatic defect) was used as a positive control. The effect of this t-TLNP-mediated fibrin rescue in restoring clot kinetics and robustness in anticoagulant-treated and platelet-depleted human blood was established by rotational thromboelsatometry (ROTEM). In these studies, normal whole blood (no platelet depletion or anticoagulation-associated hemostatic defect) was used as a positive control. Additionally, the ability of t-TLNPs to rescue fibrin in plasma with a severe hemostatic defect due to combined anticoagulation and platelet depletion was established using real-time imaging under simulated vascular flow conditions using BioFlux microfluidics imaged with inverted fluorescence microscopy. All human plasma and human blood studies described above were performed with human blood drawn from healthy donors using the Case Western Institutional Review Board (IRB) approved protocol. Finally, the hemostatic efficacy of t-TLNPs was evaluated in vivo using prophylactic administration in a tail-clip acute bleeding model in mice bearing a severe hemostatic defect due to combined anticoagulation and platelet depletion (n=3 per group), as well as emergency administration in a liver-laceration acute bleeding model in mice bearing a severe hemostatic defect due to anticoagulation (n=10 per group). The tail-clip injury model in mouse was used under protocols approved by the Case Western Reserve University Institutional Animal Care and Use Committee (IACUC), and since the “anticoagulation+platelet depletion” induced combined hemostatic defect leads to uncontrolled incessant bleeding from the tail-clip injury for 15 min and beyond, the experiment end point was considered to be at 15 min, when mice were humanely euthanized. The liver-laceration injury model in mouse was used under protocols approved by University of Pittsburgh IACUC, and here the blood loss was measured at the 20 min time point following injury and the mice were observed for an additional time (up to 3 h following injury) followed by euthanasia, organ harvesting, and histology.

### Materials

Human alpha thrombin and plasmin were obtained from Haematologic Technologies (Essex Junction, VT, USA). 1,2-Distearoyl-sn-glycero-3-phosphocholine (DSPC), 1,2-distearoyl-sn-glycero-3-phosphoethanolamine-N-[methoxy(polyethylene gly-col)₁₀₀₀] (DSPE-mPEG₁₀₀₀), 1,2-distearoyl-sn-glycero-3-phosphoetha-nolamine-N-[methoxy(polyethylene glycol)₂₀₀₀] maleimide (DSPE-PEG_{2K}-Mal), and cholesterol were purchased from Avanti Polar Lipids (Alabaster, AL, USA). Rhodamine B dihexadecanoyl-sn-glycero-3-phosphoethanolamine (DHPE-RhB) was purchased from Invitrogen (Carlsbad, CA, USA). The peptides C(GPO)₇ (CBP) and CTRYL-RIHPQSWVHQI (VBP) were purchased from Bachem (Torrance, CA, USA). Sterile saline (0.9% NaCl) was purchased from Baxter (Deerfield, IL, USA). Cellulose dialysis tubing (MWCO 100 K), calcium chloride, chloroform, methanol, dimethyl sulfoxide, apixaban, 4% paraformaldehyde, BeadBug homogenizing tubes, AlexaFluor 647 conjugated fibrinogen, calcein, collagen (equine Type I), von Willebrand factor and D-Simer Human ELISA Kits were purchased from Fisher Scientific (Pittsburgh, PA, USA). Cholesterol, calcium ionophore A23187, fibrinogen, phospholipase A₂ (bovine pancreas), and enoxaparin sodium were purchased from MilliPore Sigma (Burlington, MA, USA). Human Thrombin ELISA Kits were purchased from Abcam (Cambridge, MA, USA). The Complement C3 and Complement C3a des Arg ELISA kit was purchased from Promega Corporation (Madison, WI, USA). Econo-Column Chromatography columns were purchased from Bio-Rad (Hercules, CA, USA). G-100 Sephadex beads were purchased from GE Healthcare (Chicago, IL, USA). For ROTEM studies, all reagents were purchased from Werfen USA (Bedford, MA, USA). For platelet lumi-aggregometry studies, cuvets, stir bars, and adenosine diphosphate (ADP) were purchased from Bio/Data (Horsham, PA, USA). For BioFlux studies, the flow controller, tubings, and microfluidic plates were purchased from Fluxion Biosciences (Alameda, CA, USA). Sytox Green and glass slides were purchased from Themo Fisher Scientific (Waltham, MA). VECTASHIELD Antifade Mounting containing DAPI was purchased from Vector Laboratories (Newark, CA). The EasySep direct human neutrophil isolation kit was purchased form STEMCELL Technologies (Vancouver, Canada). For in vivo studies with mice, C57BL/6J mice were purchased from Jackson Laboratory (Bar Harbor, ME). Mouse platelet depleting anti-CD42b antibodies were obtained from Emfret Analytics (Eibelstadt, Germany). Ketamine and xylazine were obtained from Patterson Veterinary (Greeley, CO, USA).

### Manufacture and Characterization of t-TLNPs

The cysteinylated VBP and CBP peptides were conjugated to DSPE-PEG2000-Mal via a maleimide-thiol reaction to obtain DSPE-PEG_{2K}-VBP and DSPE-PEG_{2K}-CBP. These conjugates were combined at 1.25 mol % each with DSPC (41.5 mol %), cholesterol (40 mol %), DSPE-mPEG₁₀₀₀ (15 mol %) and DHPE-RhB (1 mol %) in 1/1 chloroform/methanol, and the solvent mixture was rotary-evaporated under vacuum. The resultant thin lipid film was rehydrated with a solution of 540 nM human alpha thrombin in Tris buffered saline (TBS), sonicated for 30 min, and then subsequently extruded five times through a 200 nm pore size polycarbonate filter to yield thrombin-loaded “VBP+CBP”-decorated liposomal vesicles (t-TLNPs). These t-TLNPs were passed through a column packed with TBS-swelled G-100 Sephadex beads, to remove unencapsulated thrombin, and the isolated t-TLNPs were characterized by DLS and Cryo-TEM to the measure size distribution. The “VBP+CBP”-decorated nanoparticles and control (undecorated) nano-particles were evaluated for their specific adhesion capabilities utilizing a BioFlux 200 microfluidic system (Fluxion Biosciences) and observed under inverted fluorescence microscope imaging. This system allows simulation of a physiologically relevant vascular flow environment. Here, the microfluidic channels were incubated with either 0.3 wt % bovine serum albumin (BSA) in water or 40 μg/mL equine type 1 fibrillar collagen and 10 μg/mL vWF in 20 μM acetic acid for 1.5 h. Unbound albumin and collagen were removed with a saline rinse. Next, control nanoparticles (undecorated) or targeted nanoparticles (“VBP+CBP”-decorated) were flowed over the collagen- or albumin-coated channels under a shear stress of 25 dyn/cm² for 10 min. Additionally, for some experiments control and targeted nanoparticles were first incubated in platelet-free plasma (PFP) for 60 min and then flowed over the coated channels in PFP instead of saline. Fluorescence microscopy imaging of particle Rhodamine B label was used to quantify the extent of particle adhesion to the coated surfaces. Thrombin release kinetics was measured by sealing the column-separated t-TLNPs in a MWCO 100 K dialysis bag, placing the bag in a TBS reservoir, and removing aliquots from the reservoir at predefined time points over the course of 6 h (fresh TBS was added back to the reservoir appropriately after each aliquot removal). In separate studies, similarly prepared t-TLNPs were mixed with 25 μg/mL sPLA₂, sealed in dialysis tubing, and subjected to a similar analysis. Thrombin concentrations from the collected aliquots were evaluated using a human thrombin ELISA and plotted over time to determine the release profile. The total thrombin loading was determined by adding a 1/1 methanol/chloroform mixture to t-TLNPs to induce complete particle dissolution and exhaustive release of thrombin and determining this exhaustively released thrombin concentration with ELISA. In addition to t-TLNPs, control untargeted empty nanoparticles (UNPs) were manufactured using the same methods described above, but with the lipid film rehydration performed with buffer only (i.e., no thrombin). Additionally, to characterize the particle stability under shear, experiments were performed with targeted nanoparticles loaded with carboxyfluorescein (CF) instead of thrombin. Here, the targeted particles were first flowed over “vWF+collagen”-coated microchannel surfaces to allow them to bind and become immobilized and then subjected to a flow of fresh saline at a range of shear stresses (5-50 dyn/cm²) for 30 min. The effluent was collected and transferred to a black 96-well plate in a plate reader, and the fluorescence intensity of CF (excitation 485 nm, emission 528 nm) was measured in the effluent. These data were converted to CF concentration using a standard calibration curve generated using serial dilutions of CF. Shear-induced CF release was compared to diffusive, sPLA₂-triggered, and exhaustive (induced by 1/1 methanol/chloroform) release of CF.

### Evaluation of Peptide-Decorated Nanoparticles With Healthy Endothelial Cells, Resting Platelets, Resting Neutrophils, and Complement C3

For endothelial incubation studies, microfluidic channels were fabricated by assembling three layers, including a glass slide as the bottom layer, a double-sided adhesive (DSA) film as the middle layer, and poly(methyl methacrylate) (PMMA) as the top layer. The microchannels were incubated with 0.2 mg/mL fibronectin for 1 h at 37° C. Human pulmonary microvascular endothelial cells (HPMECs, from Lonza, Basel, Switzerland) were seeded onto the microchannels and cultured with 5% CO₂ at 37° C. under a 100 μL/min flow of the medium for 48-72 h until a confluent monolayer was formed over the fibronectin-coated surface. Cultured confluent HPMEC monolayers were washed with fresh culture medium and then incubated with TNF-α at 20 μM or control nanoparticles (UNP) or targeted nanoparticles (t-LNP) particles at 4.54×10¹² particles/mL for 2 h at 37° C. The HPMECs were then rinsed again with fresh culture medium and fixed with 4% paraformaldehyde (PFA) for 15 min at room temperature. Fixed HPMECs were rinsed twice with PBS and blocked with 2% BSA for 1 h at room temperature. After washing with PBS, HPMECs were incubated with DAPI and sheep polyclonal antihuman vWF antibody (Abcam) conjugated with fluorescein isothiocyanate (FITC, 1/100 v/v dilution) for 1 h at room temperature in the dark. Images were then acquired across the microchannel at 10× using a fluorescence microscope. For platelet lumi-aggregometry, human blood was centrifuged (150 g, 15 min) to obtain platelet-rich plasma (PRP), 400 μL of PRP was added to cuvets with ADP or t-LNP, and platelet aggregation was monitored on a Bio-Data platelet aggregometer. For neutrophil studies, neutrophils were isolated from human blood using immunomagnetic separation. Isolated neutrophils (1×10⁶ cells/mL) were plated on fibrinogen-coated glass slides and allowed to adhere for 30 min. Control particles or t-LNPs were then added on the slides and incubated with the neutrophils for 1 h. Following this, the neutrophils were gently washed with saline. Similarly isolated neutrophils treated with 25 μM A23187 (calcium ionophore) were used as a positive control. The slides were then fixed with 4% PFA for 5 min, washed with saline, stained with 167 nM Sytox Green for 15 min, washed again with saline, mounted with a VectaShield mounting solution, secured with a coverslip, and imaged using a Leica Hy Volution SP8 confocal microscope. The fluorescence intensity of the Sytox Green signal was quantified using ImageJ software, as a marker of neutrophil activation and NET-osis. A Complement C3 activation assay was carried out by incubating platelet-rich plasma with saline or control (undecorated) nanoparticles or targeted (“VBP+CBP”-decorated) nanoparticles (t-LNPs) and analyzing using ELISA kits for C3 and C3a des Arg.

### Fibrin Generation Assay With t-TLNPs in Human Plasma With Induced Hemostatic Defects

The optical density (OD) based fibrin generation assay (FGA) was adapted from methods described by Curnow et al. In this assay, coagulation in plasma is initiated by CaCl₂ and the temporal OD change of the plasma due to formation and polymerization of fibrin (from plasma fibrinogen) is monitored by measuring the absorbance at 405 nm. Citrated human whole blood (WB) was centrifuged at 150 g for 15 min at room temperature to obtain platelet-rich plasma (PRP), which was further centrifuged at 2000 g for 20 min to obtain platelet-poor plasma (PPP, platelet count<50000/μL) and at 13000 g for 5 min to obtain platelet-free plasma (PFP, platelet count<5000/μL). An assay-specific coagulation buffer consisting of CaCl₂ (35 mM) and s trace amount of thrombin (1 U/ml) in TBS (66 mM Tris and 130 mM NaCl, pH 7) was prepared. In a 96-well plate, 60 μL of PPP was combined with 40 μL of buffer. Using a plate reader, absorbance values at 405 nm were recorded every 1 min for 1 h to construct the OD-based fibrin generation curves. The coagulation defect in PPP was induced by preincubating PPP with Apixaban (FXa inhibitor) at a concentration of 120 nM for 5 min before commencing the assay. We rationalized that, since FXa is a major component of the prothrombinase complex, inhibition of FXa would reduce thrombin generation and hence reduce fibrin formation. Fibrin generation curves in Apixaban-treated PPP were compared to those of PPP alone. In separate experiments, a variation of this fibrin generation assay was performed directly with PFP without Apixaban treatment. Treatments of t-TLNP, “t-TLNP+sPLA₂”, or UNP (concentration of 5.675×10¹¹ particles/mL) were added to “PPP+Apixaban” or PFP, and the resultant fibrin generations were compared to those in PPP and in PRP. This t-TLNP dose concentration was to achieve a 2.5 nM “exogenously delivered” thrombin concentration for each test condition in the assay. In both variations of the fibrin generation assay, the parameters of overall coagulation potential (OCP), onset of fibrin generation (OFG), and maximum hemostatic potential (MHP) were monitored. OCP is the total area under the OD curve and represents the total amount of fibrin formed during the experiment duration. OFG is the time at which fibrin generation begins, and MHP is the maximum absorbance value of the curve that reflects the maximum steady-state level of fibrin formed. For studies involving treatment of “PPP+Apixaban” with t-TLNP, “t-TLNP+sPLA₂” or UNP, data were presented as percent (%) deviations from PPP plasma baseline values of OCP, OFG, and MHP. For studies involving treatment of PFP with t-TLNP, “t-TLNP+sPLA₂” or UNP, data were presented as percent (%) deviations from PRP baseline values of OCP, OFG, and MHP.

### ROTEM Studies With t-TLNPs in Human Blood With Induced Hemostatic Defects

Rotational thromboelastometry (ROTEM) is a clinically established method for real-time monitoring of whole blood clotting kinetics and clot mechanical properties. In this method, 340 μL of “blood+reagents” is held in a cup and a pin suspended on a ball-bearing mechanism is lowered into the cup to rotationally oscillate through 4°75′ every 6 s with a constant force. As the blood clot forms and grows in strength, it impedes the rotation of the pin and this mechanical impedance is detected optically using a charge-coupled-device image sensor system. The CaCl₂-induced blood clotting modality (termed NATEM in ROTEM) allows real-time monitoring of this process as “endogenous clotting capability”, and any defect in the clotting mechanism (e.g., coagulation factor deficiency, platelet deficiency, etc.) results in a delay of clot formation, clot growth rate, and reduction in mechanical impedance. Therefore, this method enabled the investigation of the effect of t-TLNPs, “t-TLNPs+sPLA₂”, and UNPs on CaCl₂-induced blood clotting in human whole blood (WB) with either a coagulation defect or a platelet depletion (thrombocytopeni defect. Since the defects in thrombin generation and hence fibrin formation drastically affect the initial phases of clot growth, we monitored the clot formation time (CFT), clot growth rate (alpha angle), and clot amplitude at 10 min (A10) parameters in the ROTEM profile. Human WB was obtained from healthy donors with consent using an IRB-approved protocol. The coagulation defect was induced by preincubating WB with Apixaban (FXa inhibitor) at a concentration of 120 nM for 5 min before commencing the assay. The platelet-depleted TC Blood was made by (1) first centrifuging WB to isolating RBCs, PRP, and PFP, (2) then diluting PRP with PFP to form TC plasma containing <20000/μL of platelets, and (3) finally reconstituting the RBCs with TC plasma. The two defects (FXa inhibition and platelet depletion) were studied independently. Similarly, to the fibrin generation assay, the nano-particles (t-TLNPs and UNPs) were added at concentration of 5.67×10¹¹ particles/mL to the blood samples to achieve a 2.5 nM “exogenously delivered” thrombin concentration in each ROTEM cup. For the “t-TLNP+sPLA2” group the sPLA₂ was added with the t-TLNPs at a concentration of 25 μg/mL, and this mixture was added to the blood sample to commence the ROTEM assay. For studies involving the treatment of “WB+Apixaban” and TC Blood with t-TLNP, “t-TLNP+sPLA₂”, or UNP, data were presented as percent (%) deviations from WB baseline values of CFT, alpha angle, and A10 parameters on the ROTEM NATEM modality.

### BioFlux Microfluidic Studies of t-TLNP Effect on Fibrin Generation Under Flow in Coagulopathic Plasma

The BioFlux 200 microfluidic system observed under inverted fluorescence imaging was used to simulate a physiologically relevant vascular flow environment to study injury-site-targeted fibrin formation by t-TLNPs in coagulopathic plasma. To simulate an injury-site-relevant subendothelial matrix surface presenting vWF and collagen, micro-channels in a 24-well BioFlux plate were coated by incubating with a solution of type I equine collagen (40 μg/mL) and vWF (10 μg/mL) in 20 μM acetic acid for 1.5 h and subsequently washing off with saline. Highly coagulopathic plasma was made by combining the Apixaban-induced anticoagulation effect with the platelet depletion effect by first creating TC plasma from PRP and then incubating it with 120 nM Apixaban. We rationalized that, since in our FGA studies with human plasma as well as ROTEM studies with human blood, both Apixaban-induced FXa inhibition and platelet depletion individually led to a drastic detriment in fibrin formation that could be rescued by t-TLNPs, therefore combining these two scenarios (platelet defect+coagulation defect) would enable evaluation of the “fibrin rescue” ability of t-TLNPs in a more severe coagulopathy setting. For each channel, 500 μL of plasma (PRP or defective) with calcein-stained platelets and AlexaFluor-647 stained fibrinogen was introduced into the inlet well and then flowed over the coated channel surface at a shear stress of 25 dyn/cm² for 12 min, and “platelet accumulation+fibrin formation” was imaged under a Zeiss AxioObserver inverted fluorescence microscope with images being automatically captured every 15 s. The effect of “t-TLNPs+sPLA₂” vs “UNPs+sPLA₂” on fibrin formation in the defective plasma was studied by adding the nanoparticles (5.67×10¹¹ particles/mL concentration) with sPLA₂ (25 μg/mL) in the flow volume. After completion of the time-lapse experiments, the channels were washed with a saline flow and end point images taken. The surface-averaged fibrin fluorescence intensity (SAFI) was determined in these end-point images to be presented as “net fibrin generation” fluorescence data. Clots were then digested/lysed by flowing plasmin over them, and the lysis product was analyzed by D-Dimer ELISA to quantify the amount of cross-linked fibrin that had formed in each channel. Each condition (PRP vs defect plasma vs defect plasma with t-TLNP treatment vs defect plasma with UNP treatment) was studied for both SAFI and D-Dimer analyses in triplicate.

### Evaluation of Circulation Lifetime and Organ Biodistribution Studies of t-TLNPs in Mice

Mice were anesthetized (using 2% isoflurane gas) and retro-orbitally injected with 125 μL of Rhodamine B-labeled nanoparticles (NPs) at a concentration of 1.14×10¹² particles/mL and allowed to recover. After 1, 6, 12, or 24 h, the mice were anesthetized and underwent a midline laparotomy, and blood was collected from the inferior vena cava (IVC). Livers, lungs, kidneys, spleens, and hearts were excised and placed in pre-weighed homogenizing tubes. The samples were then freeze-dried and their dry weights recorded. The dry organs were homogenized with a BeadBug Microtube Homogenizer. The blood and homogenized organs were then mixed vigorously with 1/1 methanol/chloroform to disassemble the Rhodamine B labeled lipids of nanoparticles in the samples. The samples were then centrifuged (20 min, 12000 g) to separate the organ tissue from the supernatant containing Rhodamine B labeled lipids. The supernatant was analyzed using a Biotek Synergy H1 Plate Reader (λₑₓ=560 nm, λₑₘ=580 nm) to assess Rhodamine B fluorescence. The biodistribution of the nanoparticles was determined by calculating the percentage of injected dose per organ utilizing an appropriate calibration curve that correlated the RhB fluorescence intensity with the particle concentration.

### Evaluation of t-TLNPs in Tail-Clip Bleeding Model in Severely Coagulopathic Mouse

All experiments were carried out in accordance with protocols approved by the CWRU IACUC. Wild-type C57/BL6 mice (average weight 20 g) were injected intra-peritoneally with anti-CD42b (anti-GPIbα) antibody at a dose of 0.5 μg/kg. The normal platelet count is mice is approximately 1500/nL and upon a platelet-depleting anti-CD42b antibody dose, thrombocytopenia TC (<500 platelets/nL) was confirmed 18-24 h later, by drawing blood retro-orbitally and measuring the platelet count using a Heska HemaTrue system. After the confirmation of TC mice, these mice were further dosed with low-molecular-weight heparin (LMWH or Enoxaparin at 12.3 mg/kg) to induce an additional anticoagulant effect. After 1 h, a tail-clip was done on normal (no TC and anticoagulation) and defect (TC+anticoagulation) mice by transecting 3 mm from the tail tip with a surgical scalpel and the clipped tail was immediately immersed in warm (37° C.) saline. Bleeding was monitored over time, and if bleeding did not stop beyond 15 min, then the 15 min time point was considered as the experiment end point. In separate experiments, t-TLNPs or UNPs (stock concentration of 1.14×10¹² particles/mL) were administered retro-orbitally in “defect mice” 15 min before tail-clip and bleeding was similarly monitored by immersing the injured tail in warm saline. In all mice experimental groups, the bleeding time (time for bleeding to stop) was recorded as a percentage (%) of 15 min. At the 15 min time point, the collected blood was analyzed for hemoglobin by the sodium lauryl sulfate method using UV spectrometry measurements at 550 nm, and these data were used to calculate blood loss. The t-TLNP particle administration volume was calculated to achieve an initial encapsulated circulating concentration of 1 nM thrombin or an exogenous thrombin dose of 0.031 mg/kg per animal.

### Evaluation of t-TLNPs in Traumatic Liver Injury Model in Severely Coagulopathic Mouse

C57BL/6J mice, 8-9 weeks old, were anesthetized using 70 mg/kg of sodium pentobarbital via an intraperitoneal injection. The femoral artery and vein were cannulated for hemodynamic monitoring and administration of intravenous treatment, respectively. Thirty (30) minutes prior to injury, mice were treated intravenously with 1 U/g of unfractionated heparin (UFH), followed by a flush of normal saline of equal volume to ensure complete infusion, through a catheter placed in the femoral vein. Then, mice were subjected to a previously validated model of uncontrolled hemorrhage that utilizes liver laceration. Briefly, following a midline laparotomy incision, pre-weighed absorption triangles were inserted inside the abdominal cavity against the abdominal wall without touching the liver, then the right middle lobe of the liver was lacerated. The resected liver weight was recorded in g immediately after. Treatment (sham saline or UNP or t-TLNP) was administered via a femoral vein catheter, 1 min after the liver laceration. The abdominal cavity was stapled close. Twenty (20) min following the injury and postinjury treatment, the abdomen was reopened and the absorption triangles were retrieved and weighed. Blood loss was calculated as the difference between the pre- and post-liver laceration weights of the absorption triangles and recorded in grams (g). The abdominal cavity was then closed, and the mice were observed for 3 h from the time of liver laceration. At the 3 h time point, the mice were sacrificed, and their organs were harvested for histological staining and imaging.

### Statistical Analysis

All data from the in vitro fibrin generation, ROTEM, and BioFlux microfluidic assays, as well as blood loss data from the in vivo tail-clip and liver-laceration injury studies, were analyzed using one-way ANOVA with Tukey's multiple comparisons test. For tail-clip studies, the bleeding time was assessed with a Log-rank (Mantel-Cox) test and Gehan-Breslow-Wilcoxon test. In all analyses, significance was considered to be p<0.05. For all data shown, *p≤0.05, **p≤0.01, ***p≤0.001, and ****p≤0.0001.

### Results

### Targeted Thrombin-Loaded Lipid Nanoparticle (t-TLNP) Manufacture and Characterization

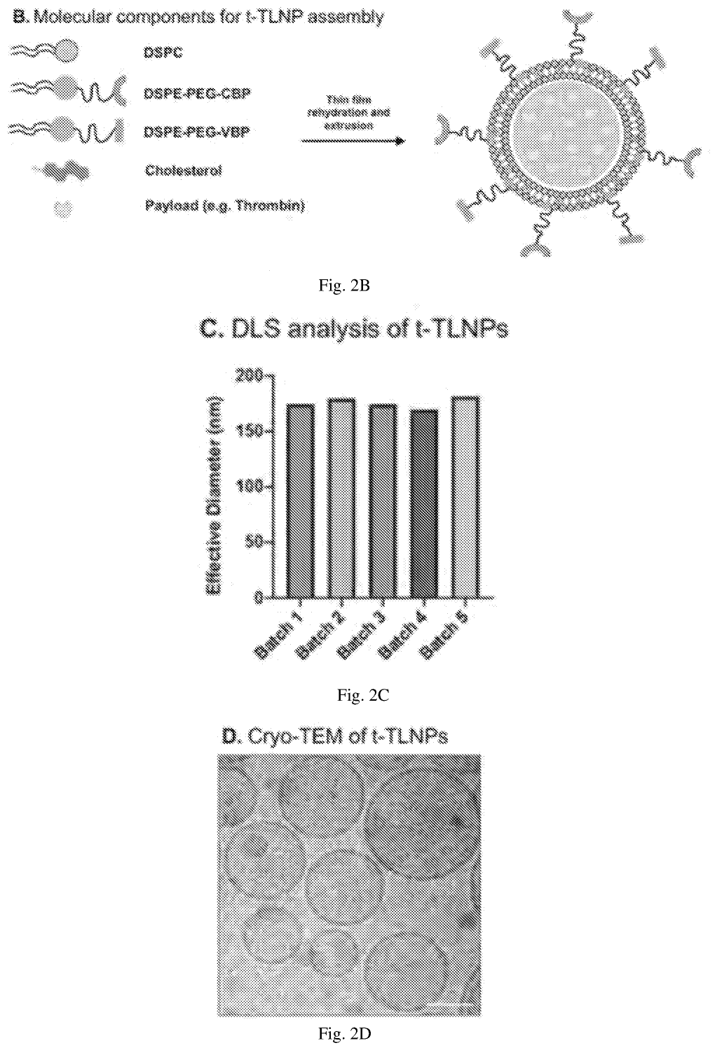

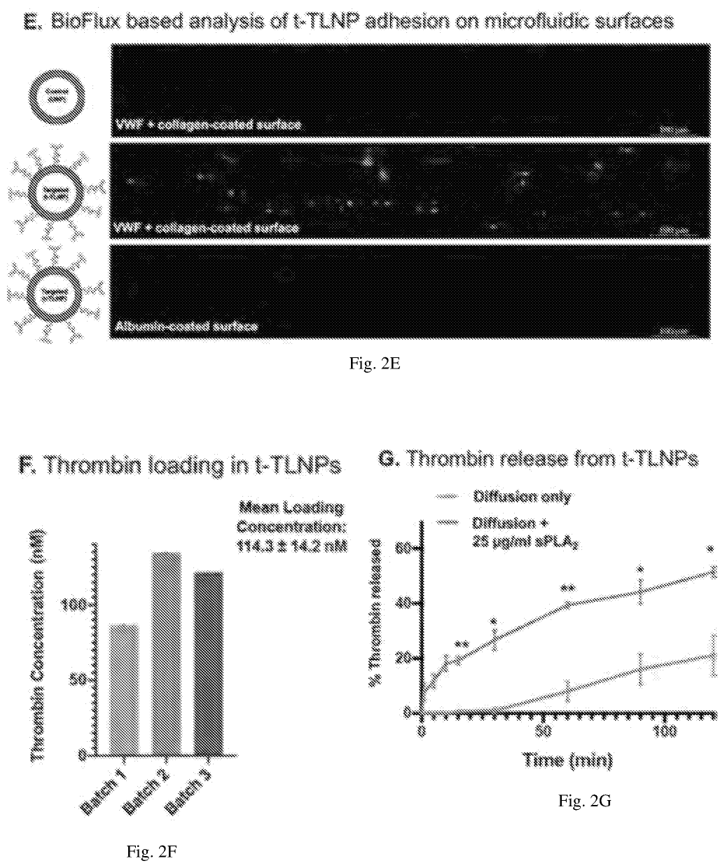

For targeting to an injury site via platelet-inspired mechanisms, the liposomal LNPs were surface-decorated with vWF-binding peptides (VBPs) and collagen-binding peptides (CBPs). The VBP sequence TRYLRIHPQSWVHQI (SEQ ID NO: 1) was chosen from a peptide library that mimicked FVIII C2 domain sequences with binding specificity to the D′-D3 domain of vWF. The CBP sequence [GPO]₇ (SEQ ID NO: 2) was chosen on the basis of reports of high helicogenic affinity of this sequence to fibrillar collagen, and such sequences have been used for molecular imaging of collagen fibers. Both VBP and CBP sequences were cysteinylated to enable thiol-mediated conjugation to maleimide-terminated lipid molecules for subsequent incorporation into liposomal LNPs. FIG. 2A depicts the chemical structures of lipid-peptide conjugation reactions for t-TLNP manufacturing, and FIG. 2B depicts the molecular components used in t-TLNP assembly. In current studies, the total “DSPE-PEG-VBP+DSPE-PEG-CBP” composition in t-TLNPs was kept at 2.5 mol % of total lipid per batch of particle manufacture, with a 1:1 ratio (1.25 mol % of each).

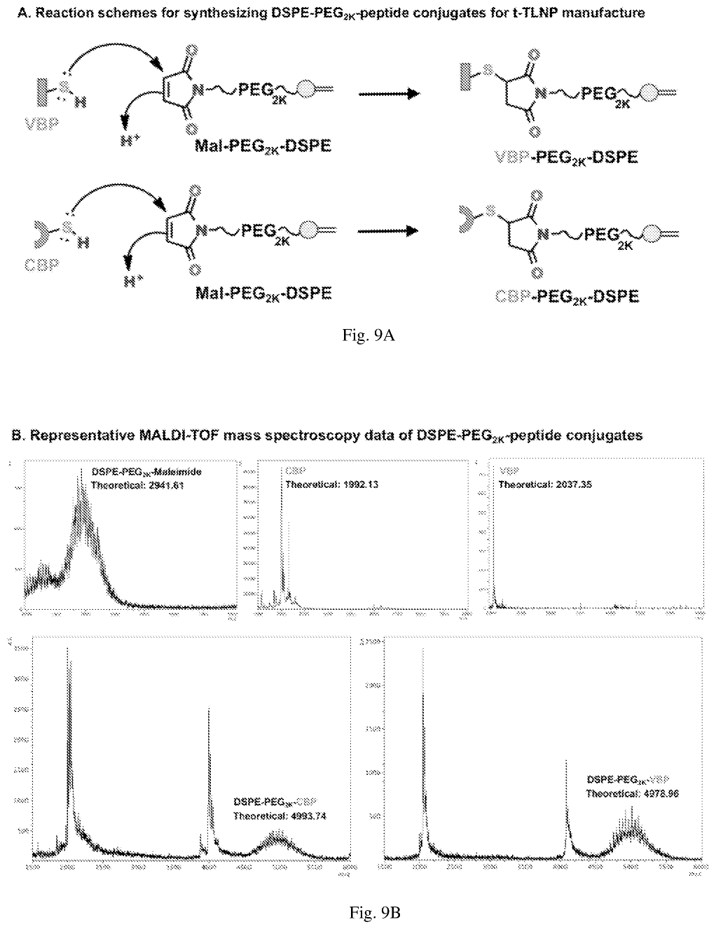

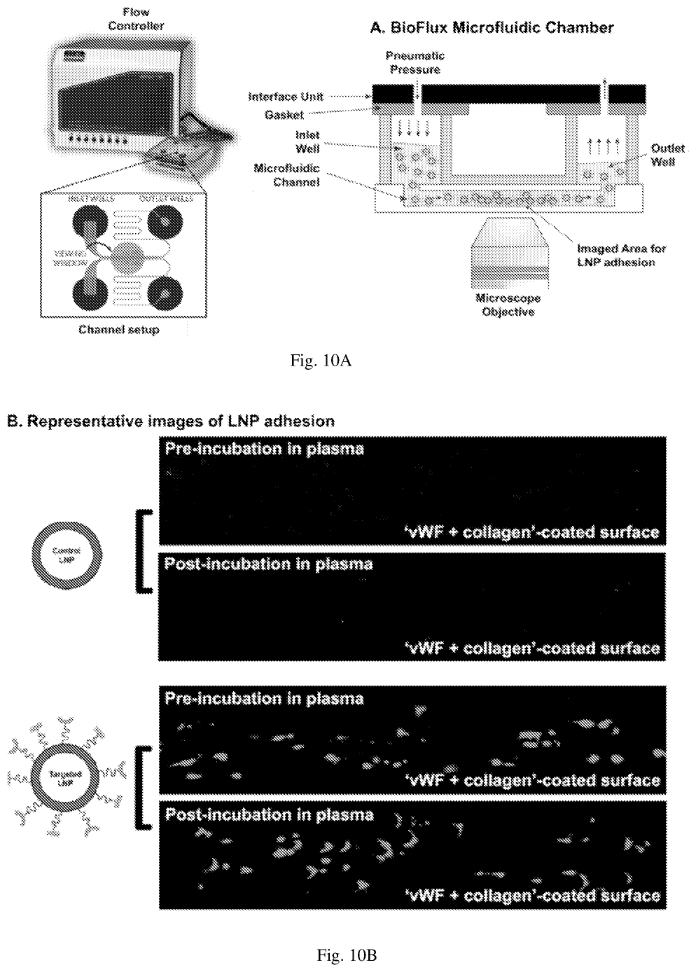

FIG. 9 shows the maleimide-thiol chemistry and representative mass spectroscopy characterization data for the DSPE-PEG-peptide conjugates. FIG. 2C shows dynamic light scattering (DLS) data of size distribution over five representative batches of “VBP+CBP”-decorated LNPs. FIG. 2D shows a representative cryo-transmission electron microscopy (Cryo-TEM) image of “VBP+CBP”-decorated LNPs. As is evident from these analyses, the nanoparticles could be manufactured reproducibly with a consistent particle diameter of about 175 nm. The nanoparticles thus manufactured were evaluated for their specific adhesion capability on the injury-site-relevant “vWF+collagen” surface under a hemodynamically relevant flow environment using a BioFlux microfluidic setup. For these studies, Rhodamine B (RhB, red fluorescence) labeled control nanoparticles (undecorated liposomes) and targeted nanoparticles were suspended in saline and flowed over “vWF+collagen”-coated microfluidic channels at 25 dyn/cm² shear stress. Additionally, the targeted nanoparticles in saline were also flowed over a “negative control” surface, namely albumin-coated microchannels, since VBP and CBP are not expected to have any adhesion specificity to albumin. The experimental setup is shown in FIG. 10A. As shown in representative images in FIG. 2E, control nanoparticles were unable to bind to the “vWF+collagen”-coated surface while the “VBP+CBP”-decorated nanoparticles showed a high degree of adhesion on this surface. Additionally, these targeted nanoparticles did not show any adhesion to the albumin-coated surface. The results in FIG. 2G as well as in these movies clearly indicate that the t-TLNPs have specific binding ability to vWF and collagen via VBP- and CBP-mediated interactions, respectively.

Additional studies were done to assess whether the thiol-maleimide reaction mediated conjugation of VBP and CBP on t-TLNPs remains stable in plasma. This is because, although the thiol-maleimide mediated bioconjugation is an established approach in the pharmaceutical field, it has been reported that in vivo some instability may occur in such bioconjugated molecules due to retro-Michael reactions with serum thiols. For these studies, control nanoparticles and “VBP+CBP”-decorated nanoparticles were incubated in human platelet-free plasma (PFP) for 60 min, and then these particles in the plasma suspension were flowed over the “vWF+collagen”-coated surface in the BioFlux channels. FIG. 10B shows representative images from these studies, indicating that the control nanoparticles were unable to bind to the “vWF+collagen”-coated surface irrespective of plasma incubation, while the targeted nanoparticles retained their ability to bind to such surface even after incubation with plasma. These results indicate that the thiol-maleimide reaction mediated VBP and CBP decorations on the targeted nanoparticle surface remain mostly stable in plasma and retain their specific binding ability to vWF and collagen, respectively.

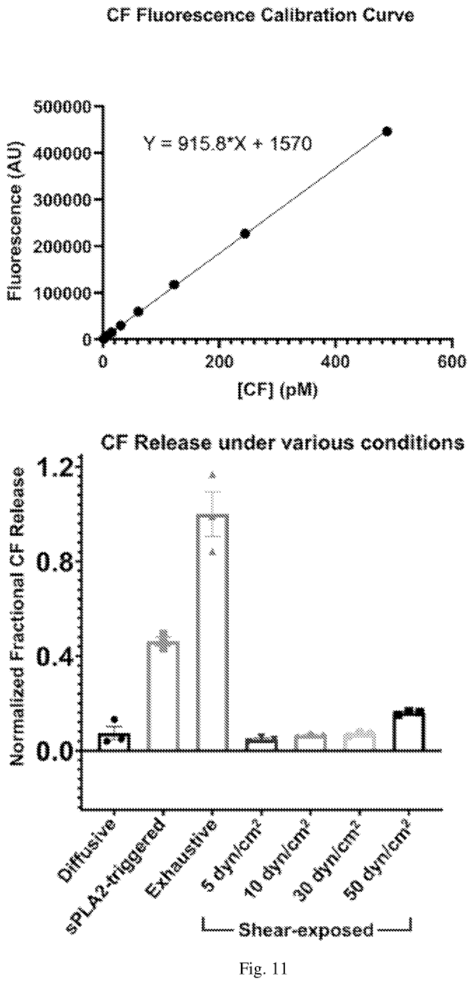

FIG. 2F shows thrombin-loading data for three representative batches of t-TLNPs, demonstrating that thrombin could be loaded at an average concentration of 114.3±14.2 nM. FIG. 2G shows the thrombin release profile from t-TLNPs in PBS at 37° C., in the absence versus the presence of sPLA₂ over the course of 2 h. The diffusive release of thrombin from the t-TLNPs was slow (only ˜20% released over 2 h, magenta line), while in the presence of sPLA₂ the release was significantly enhanced (˜60% released over 2 h, green line). The release rate of thrombin from t-TLNPs was also increased in the presence of sPLA₂. These data strongly suggest that sPLA₂ can accelerate the release of thrombin from t-TLNPs and that this property can be advantageous for enhanced thrombin release from injury-site-anchored t-TLNPs, since activated platelets and macrophages upregulate sPLA₂ secretion at a vascular injury site. Additionally, we studied whether shear forces relevant to the circulation environment could destabilize the t-TLNPs, resulting in increased payload release, because this can be a potential systemic prothrombotic risk from off-target thrombin release in vivo. For this, we loaded carboxyfluorescein (CF) as a model payload in the targeted LNPs. Of note, CF loaded at a high concentration in the particle core remains self-quenched, giving a low fluorescence signal, but as CF is released it gets diluted and the fluorescence signal is enhanced, providing a way to use fluorescence spectrometry to measure payload release. For comparison studies, first the CF-loaded targeted LNPs were added to saline in a well plate and CF fluorescence was monitored for diffusive release, release triggered by sPLA₂ added to a well, and exhaustive release due to complete destabilization of the particles by incubating with 1/1 methanol/chloroform mixture. Subsequently, the CF-loaded targeted LNPs were incubated on “vWF+collagen”-coated microchannels, the resultant bound CF-loaded LNPs were exposed to flow of saline at low to high shear (5-50 dyn/cm²) for 20 min in the BioFlux system, and the effluent from such experiments in the outlet well of the BioFlux setup was analyzed for “released” CF fluorescence. As shown in the data in FIG. 11, in comparison to sPLA₂-triggered release and chloroform/methanol-induced exhaustive release, exposure to shear caused very minimal CF release, indicating that the particles are quite stable even under high shear (50 dyn/cm²). On consideration that physiological shear forces in venous and arterial circulation are usually in the range of 5-30 dyn/cm², it can be rationalized that thrombin loaded in such LNPs will have minimal release under physiological shear in circulation and will not pose a high systemic thrombotic risk. Once the particles bind to the target injury site, the thrombin release is expected to occur by diffusion and sPLA₂-triggered destabilization, and if higher shear at the injury site induces more thrombin release, that could be further beneficial to site-specific hemostatic augmentation.

### Evaluation of Biosafety Characteristics of Peptide-Decorated Nanoparticles

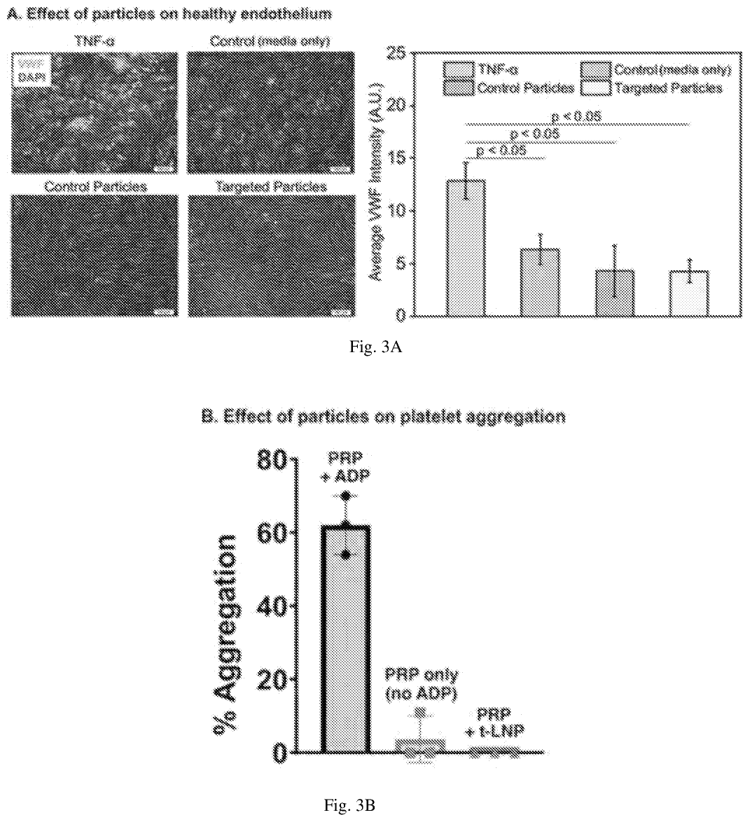

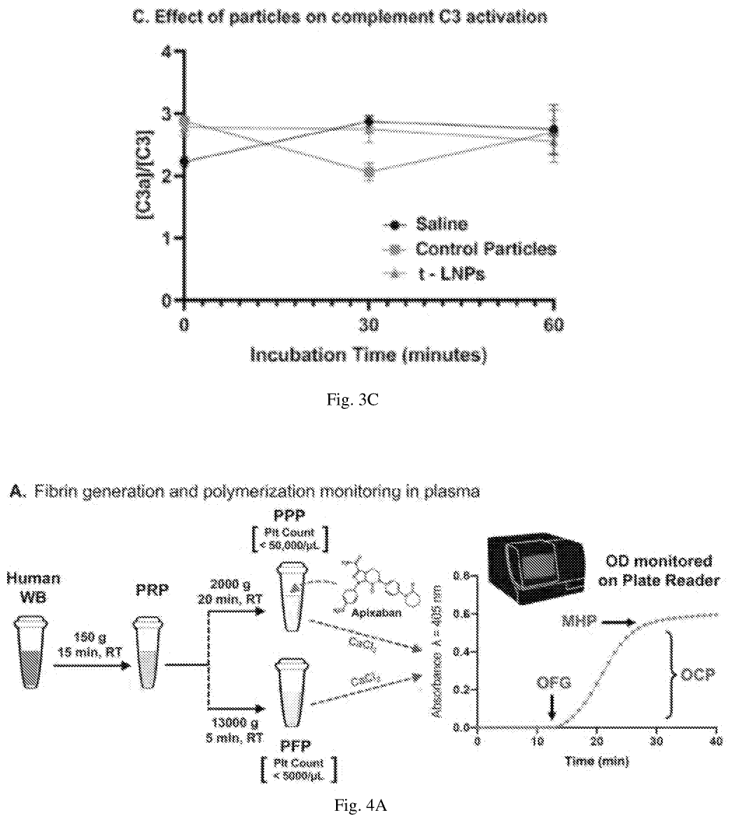

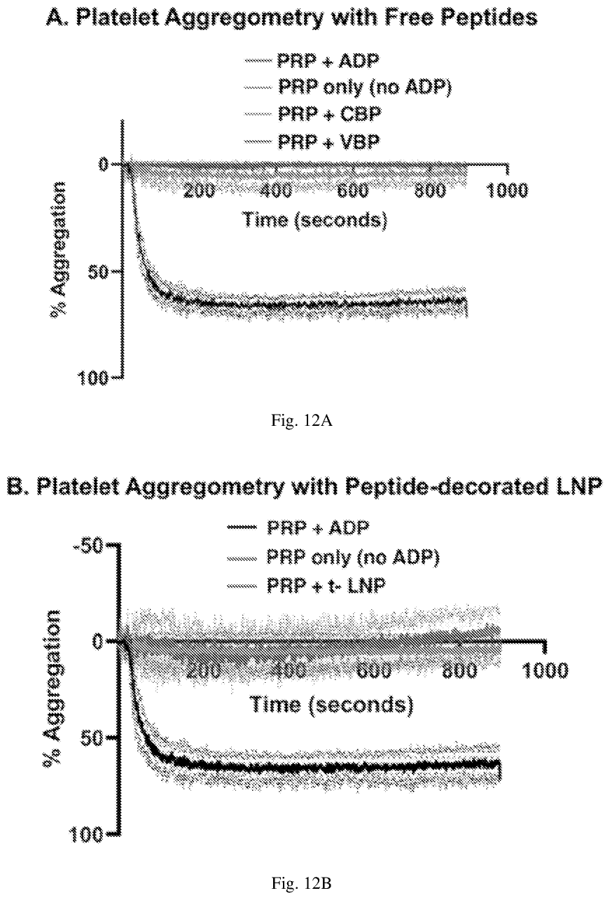

The “VBP+CBP”-decorated nanoparticles were evaluated for biosafety characteristics using four complementary assays. In the first assay, it was evaluated whether these nanoparticles activate healthy endothelial cells, because this would indicate potential safety concerns toward healthy vasculature in vivo. For these experiments, human pulmonary microvascular endothelial cells (HPMEC) were cultured in custom-made microfluidic channels coated with 0.2 mg/mL of fibronectin for 1 h at 37° C. and maintained with 5% CO₂ at 37° C. under 100 μL/min media flow for 48-72 h until a confluent monolayer was formed. As a “positive control” for EC activation, this EC monolayer was exposed to TNF-α (20 μM) and immunostaining of vWF expression on these stimulated ECs was used as an activation marker. In analogous experiments, similar EC monolayers were exposed to incubation of “VBP+CBP”-decorated particles or control undecorated particles (4.54×10¹² particles/mL for 2 h at 37° C.), and the vWF expression was similarly imaged. As shown in FIG. 3A, in comparison to TNF-α treatment, neither the peptide-decorated particles nor the control particles were found to activate ECs (minimal vWF staining). This indicates that the nanoparticles do not interact with and activate healthy ECs. In another assay, the VBP and CBP peptides as well as “VBP+CBP”-decorated nanoparticles were evaluated using platelet lumi-aggregometry to assess whether they activate and aggregate resting platelets, because this would indicate the risk of systemic thrombosis. As a positive control, adenosine diphosphate (ADP) was used as a platelet agonist which causes substantial platelet activation and aggregation. The results from these studies are shown in FIG. 3B, with raw aggregometry profiles being shown in FIGS. 12A,B. As is evident from these data, neither the free VBP and CBP peptides nor the “VBP+CBP”-decorated nanoparticles (labeled as targeted LNPs or t-LNPs) were found to cause any platelet activation and aggregation in PRP (aggregation percent similar to that of PRP without ADP). In a third assay, the “VBP+CBP”-decorated nanoparticles (t-LNPs) were evaluated via ELISA for their ability to activate complement C3 to C3a, as this would indicate a systemic complement-mediated immunological risk. As is shown is FIG. 3C, the C3a/C3 ratio in plasma remained similar to the baseline (saline), on incubation with control nanoparticles or t-LNPs, indicating that in vivo these particles would have minimal complement activation risks. In a fourth assay, the control particles as well as t-LNPs were evaluated for their ability to activate neutrophils, because this would indicate an unwanted stimulation of the innate immune defense mechanism. Neutrophils, when they are stimulated, exhibit a specific mechanism of deconvoluting their DNA via histone citrullination and extruding the DNA as neutrophil extracellular traps (NETs) which can be stained by Sytox Green. Therefore, for these studies, the calcium ionophore A23187 was used as a positive control, as it is well-known to induce NET formation. As shown in representative images (DAPI, blue nuclei of neutrophils; Sytox Green, NETs) and quantitative data shown in FIG. 12C, in comparison to A23187 neither control nanoparticles nor t-LNPs were found to stimulate neutrophils, indicating potential safety toward the innate immune system. Altogether, results from these four complementary bioassays strongly suggest that the “VBP+CBP”-decorated nanoparticles are expected to have an inherently safe profile in circulation.

### t-TLNPs Restore Fibrin Generation Capability in Anticoagulant-Treated and Platelet-Depleted Human Plasma

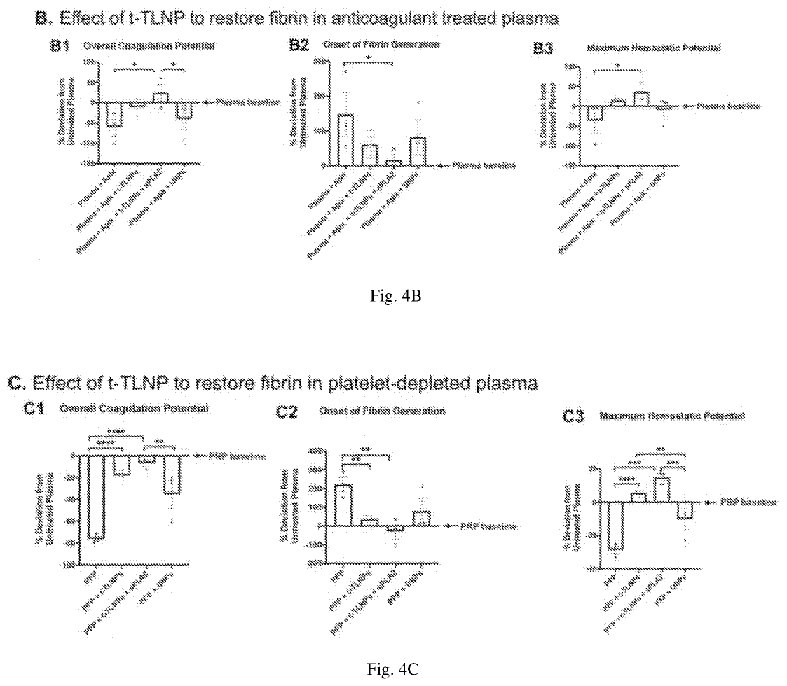

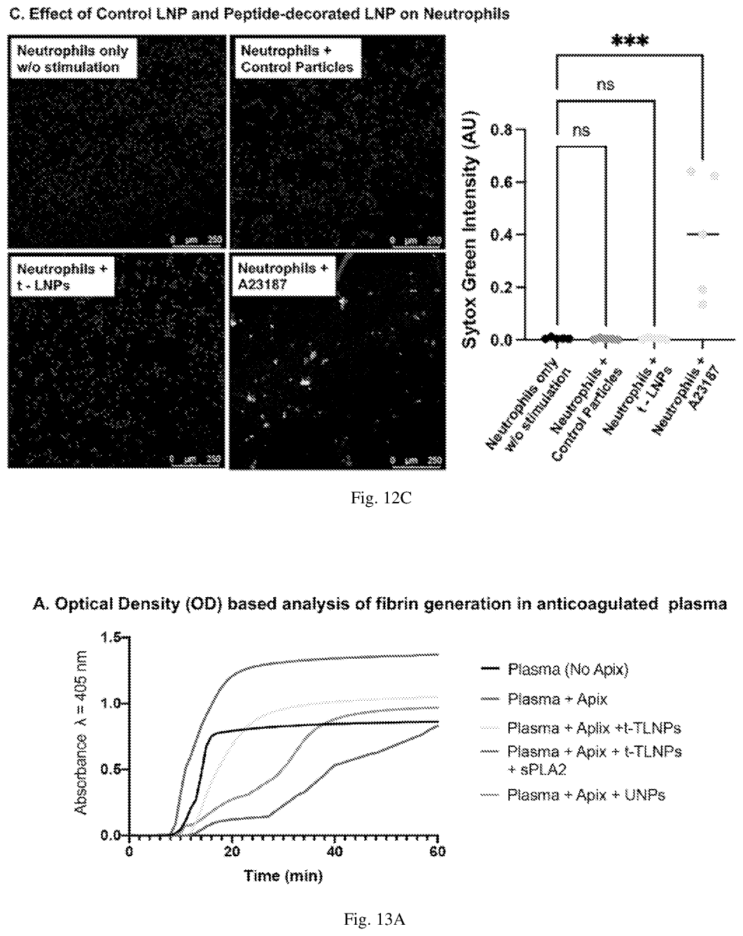

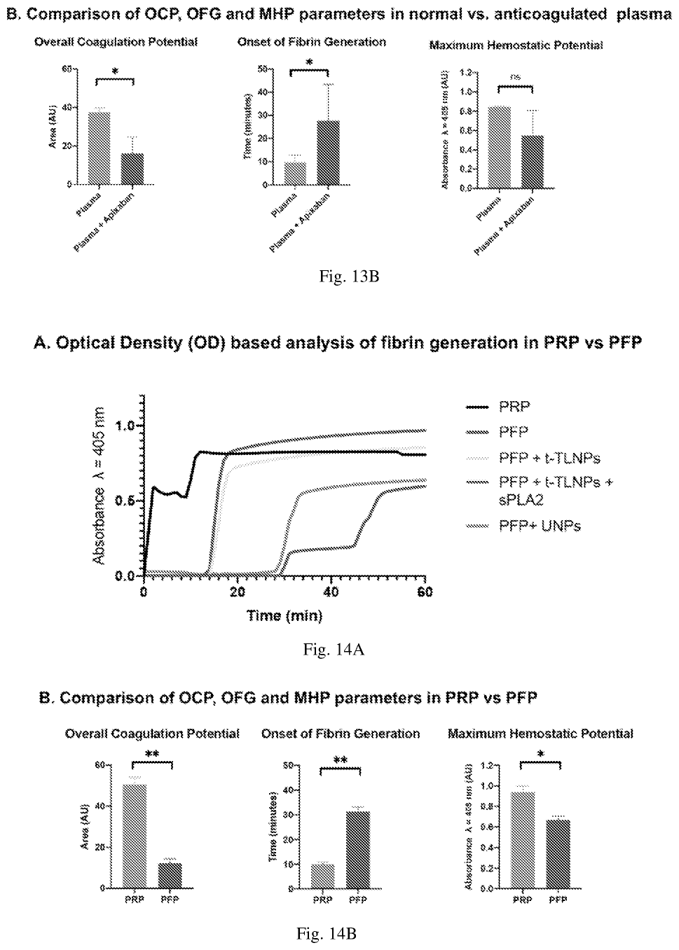

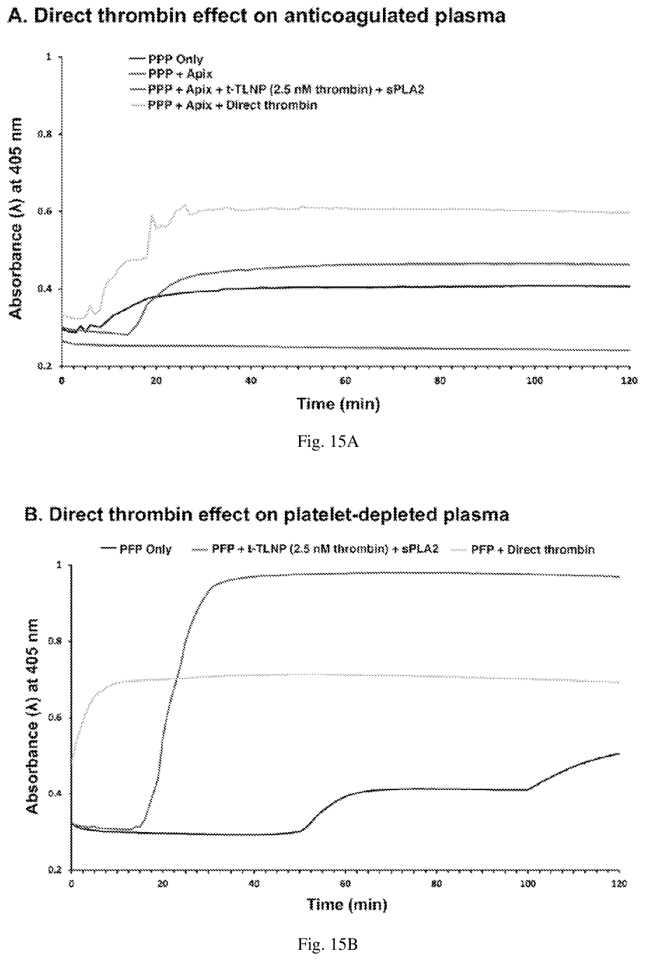

Since our in vitro characterization studies demonstrated that t-TLNPs can release thrombin diffusively as well as at enhanced levels triggered by sPLA₂, we investigated whether such thrombin release from t-TLNPs can restore fibrin generation in human plasma in settings of a clinically relevant “hemostatic defect”. On the basis of the hemostatic mechanism of thrombin generation depicted in FIG. 1, either inhibition of coagulation factor (e.g., FXa) or depletion of platelets as a whole (hence reducing the availability of the PS-rich procoagulant platelet surface) is expected to reduce overall thrombin production and thereby reduce the corresponding fibrin generation. Therefore, these are the two characteristic “hemostatic defects” that were induced in human plasma (FIG. 4A) and t-TLNPs were evaluated for their ability to rescue/restore fibrin generation via direct release of thrombin. The fibrin generation kinetics in plasma was studied using spectrophotometric measurements (absorbance at 405 nm) of the optical density (OD) change in plasma due to fibrin generation and polymerization and thus monitoring the overall coagulation potential (OCP), onset of fibrin generation (OFG), and maximum hemostatic potential (MHP) parameters. The raw data showing fibrin generation curves and changes in these parameters due to anticoagulant effect and platelet depletion effect are shown in FIGS. 13 and 14. Additionally, in vitro data in FIG. 15 show that, in such anticoagulated or platelet-depleted plasma, directly adding thrombin (positive control) is able to restore fibrin generation, but such a direct administration of thrombin cannot be used intravenously in vivo due to systemic coagulation risks. For the various defects and t-TLNP treatment effects we present the parametric results in FIG. 4B, C as percent (%) deviations from the healthy plasma baseline, since the goal was to evaluate whether t-TLNP treatment could restore the OCP, OFG, and MHP parameters in defective plasma to be closer to the healthy plasma baseline values. Treatment with empty (unloaded) nanoparticles (UNPs) was used as a control. As shown in FIGS. 4B1-B3, treatment of human plasma with Apixaban (an FXa inhibitor) induced a substantial debilitation (negative percent deviation for OCP and MHP, positive percent deviation for OFG) of fibrin generation/polymerization (a black curve for healthy plasma vs a blue curve for Apixaban-treated plasma are shown in representative OD curves and corresponding values in FIG. 13). Treatment with t-TLNPs, but without sPLA₂ addition, resulted in partial restoration of OCP and MHP parameters toward healthy (i.e., Apixaban-free) plasma baselines but not as much of an improvement in OFG, indicating some recovery in fibrin generation/polymerization over the 60 min assay period due to diffusive release of thrombin over this time. When the Apixaban-treated plasma was treated with “t-TLNP +sPLA₂” to simulate “enzyme-triggered thrombin release”, the OCP, OFG, and MHP parameters were all significantly restored toward the “healthy plasma” baseline. Treatment with UNPs was unable to achieve such a restoration of fibrin generation/polymerization parameters. Representative raw OD data in FIG. 13A clearly show the improvement of fibrin generation when anticoagulated plasma (deep blue curve) is treated with t-TLNP (cyan curve), which have shifted toward the black curve (healthy plasma) and purple curve (t-TLNP+sPLA₂ treatment) performing better than the black curve (healthy plasma). This demonstrates rescue of fibrin generation in comparison to the deep blue curve (Apixaban-treated plasma) and red curve (UNP treatment). Overall, these studies indicate that t-TLNPs can potentially restore fibrin generation kinetics as well as amount, in scenarios where coagulation factor dysfunction (simulated by FXa inhibition here) induces a hemostatic defect.

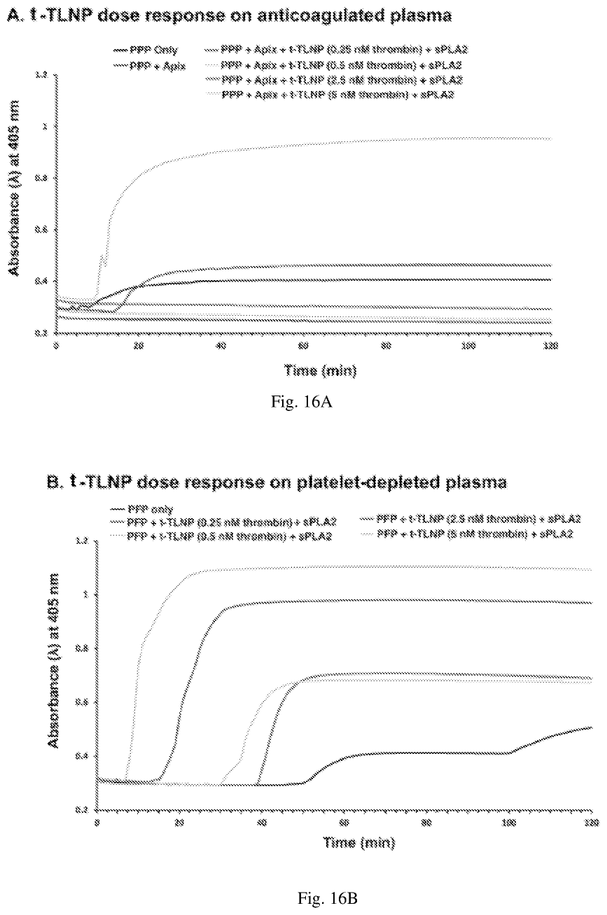

FIGS. 4C1-C3 (and corresponding raw OD data and quantitative comparison in FIG. 14) show the detrimental effect of extreme platelet depletion (going from platelet-rich plasma or PRP to platelet-free plasma or PFP) on fibrin OCP, OFG, and MHP parameters and to what extent t-TLNPs are able to restore them. In comparison to the PRP baseline, PFP showed substantial debilitation (negative percent deviation for OCP and MHP parameters, positive percent deviation for OFG parameter) of fibrin (black curve for PRP vs deep blue curve for PFP in FIG. 14). Treatment of PFP with t-TLNPs significantly restored OFG closer to the PRP baseline and led to significant recovery in OCP and MHP toward PRP levels (raw OD data as the cyan curve in FIG. 14A). Adding sPLA₂ to t-TLNP treatment in PFP led to a further improvement in OFG, OCP, and MHP parameters (raw OD data as the purple curve in FIG. 14A). UNPs were unable to recover any such parameters in PFP (raw OD data as the red curve in FIG. 14A). Additional in vitro studies were carried out to assess whether t-TLNPs can have a dose response effect on fibrin generation in Apixaban-treated (i.e., anticoagulated) or platelet-depleted plasma. For these studies, the same fibrin generation assay in anticoagulated plasma or PFP was carried out with various doses of t-TLNPs that correspond to various doses of encapsulated thrombin (0.25-5 nM). Representative results from these studies are shown in FIG. 16, which indicate that indeed the modulation of t-TLNP dose (and hence encapsulated thrombin dose) can allow for modulation of fibrin restoration response, in both anticoagulated plasma and platelet-depleted plasma. Overall, these studies indicate, that even in an extreme platelet-depleted scenario (e.g., severe hemorrhage, severe thrombocytopenia, etc.), t-TLNPs can directly deliver thrombin to potentially rescue fibrin kinetics and restore hemostasis.

### t-TLNPs Enhance Viscoelastometric Characteristics of Clots in Anticoagulant-Treated and Platelet-Depleted Human Whole Blood

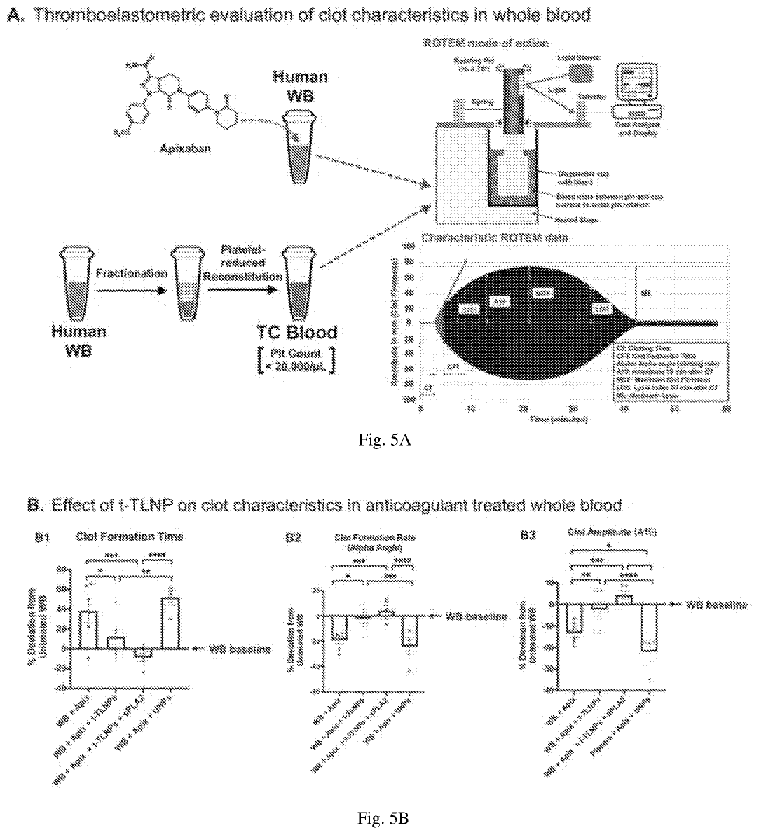

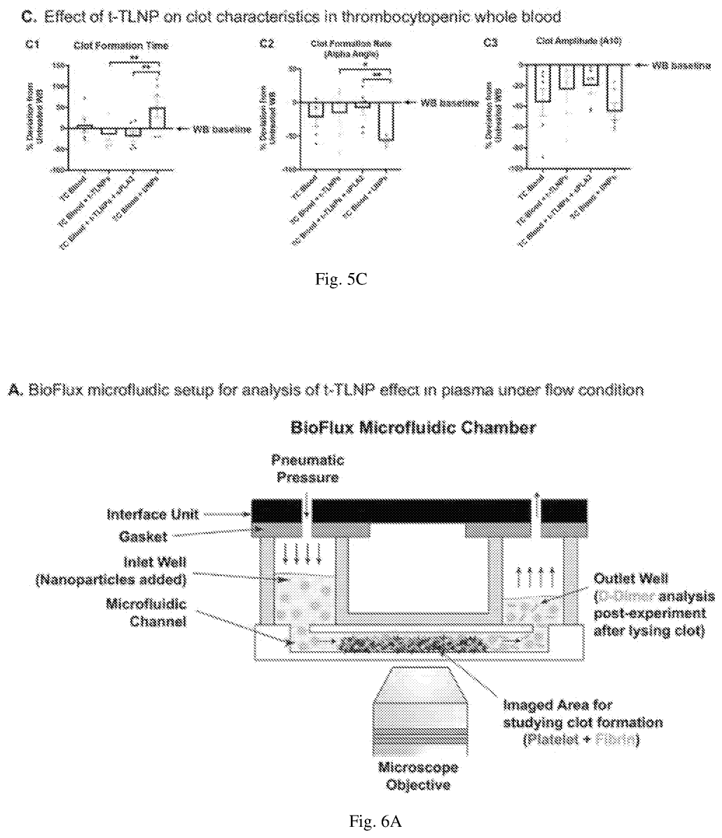

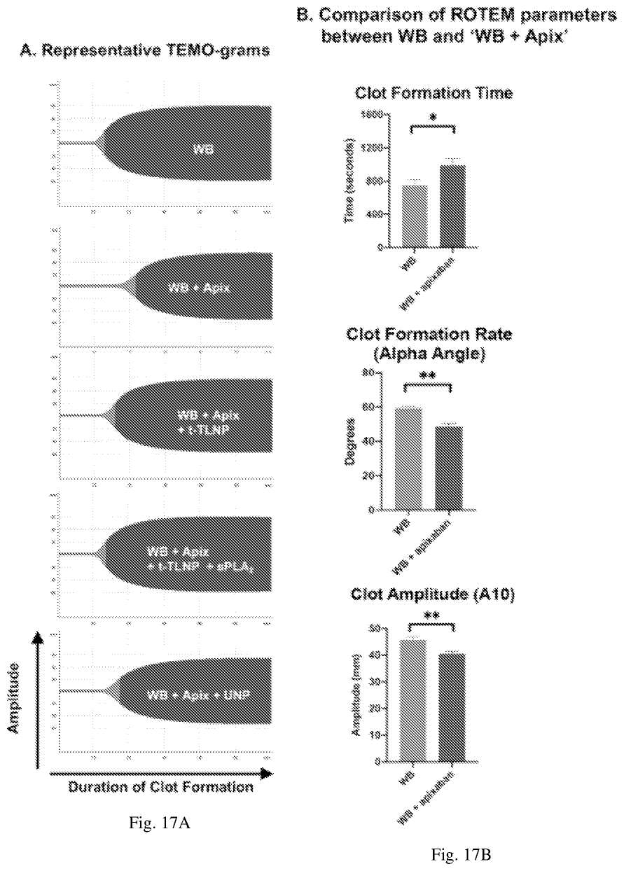

On the basis of the finding that t-TLNPs can restore fibrin generation and polymerization in anticoagulant-treated as well as platelet-depleted human plasma, we studied whether this capability of t-TLNPs can enhance the viscoelastometric properties of clots in human whole blood with similar anticoagulation-induced as well as platelet depletion-induced defects. For these studies the rotational thromboelastometry (ROTEM) methodology was used (FIG. 5A), since ROTEM-based diagnostics have become clinically significant in the assessment of hemostatic defects in trauma and surgery. To induce an anticoagulation effect, human whole blood was directly pretreated with Apixaban (FXa inhibitor). To create platelet-depleted (thrombocytopenialood, the whole blood was first fractionated into its components (RBC, leukocytes, platelets, plasma) and then reconstituted with a low (<20000/μL) platelet count. The resultant blood samples were subjected to ROTEM analysis in NATEM mode (CaCl₂-induced clotting), and the clot formation time (CFT), clot formation rate (alpha angle), and early amplitude at 10 min (A10) were recorded. The rationale was that debilitation in thrombin generation due to an anticoagulant effect as well as a platelet depletion effect is expected to reduce the early kinetics of clot formation and also possibly affect the firmness (amplitude) of the growing clot. Treatment of such anti-coagulated or platelet-depleted blood samples with t-TLNPs or “t-TLNPs+sPLA₂” was studied to see whether this restores the clot kinetics and amplitude. Treatment with unloaded (empty) nanoparticles (UNPs) was used as a negative control. As before for fibrin generation studies in plasma, for the ROTEM studies on the various defects and t-TLNP treatment effects on whole blood we present the ROTEM parametric results in FIG. 5B, C as percent (%) deviations from the healthy whole blood (WB) baseline, since the goal was to evaluate how close to the healthy WB baseline values the parameters could be restored upon t-TLNP treatment. As is evident from FIGS. 4B1-B3 (corresponding raw TEM-ograms and quantitative comparison are shown in FIG. 17) Apixaban-treated WB showed significantly prolonged CFT, reduced alpha angle, and reduced A10 in comparison to healthy WB, indicating slower clot formation and clot growth, as well as weaker clot quality.

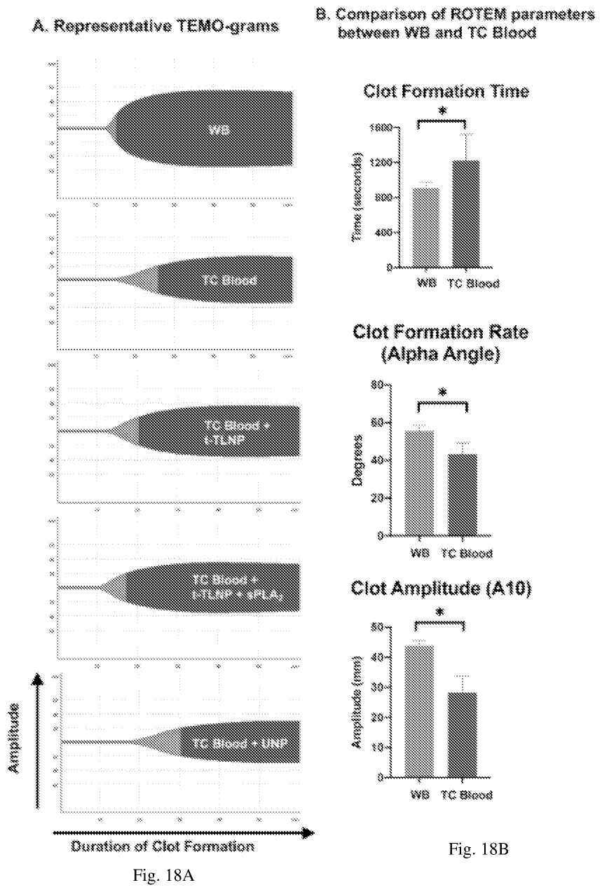

Treatment of this anticoagulated blood with t-TLNPs but without sPLA₂ significantly improved these parameters, reducing CFT and increasing alpha angle as well as A10 values toward WB baseline levels. These parameter improvements were further amplified when t-TLNPs and sPLA₂ were added together, suggesting that diffusive as well as sPAL₂-triggered release of thrombin from t-TLNPs could directly generate fibrin from fibrinogen to restore clot kinetics and clot robustness, even when the native prothrombinase activity is inhibited in the blood (e.g., by FXa inhibition here). UNP treatment did not indicate any such improvement and in fact worsened the parameters further, possibly due to a dilution effect. FIGS. 5C1-C3 (and corresponding raw data in FIG. 18) show the detrimental effects of the aforementioned ROTEM parameters in thrombocytopeniaod (TC blood). The mean CFT of TC blood was higher, and the mean alpha angle and A10 of TC blood were lower than those of healthy WB, suggesting an impairment in clot kinetics and robustness. Treatment of the TC blood with t-TLNPs as well as “t-TLNPs+sPLA₂” was found to partially improve the CFT, alpha angle and A10 characteristics, but not at statistically significant levels in comparison to TC blood parameters. Treatment with UNPs did not show any such improvement trend. These results indicate that severe depletion of platelets (reducing the count from the normal ˜200000/μL to <20000/μL) creates more severe debilitation of clot characteristics in comparison to coagulation factor inhibition, and while the t-TLNPs are highly efficient in restoring clot viscoelastometric characteristics in a “coagulation inhibition” setting, in a “severe platelet depletion” setting the t-TLNPs can only partially restore these characteristics.

### t-TLNPs Enhance Fibrin Generation Under Simulated Vascular Flow Environment Imaged in Real-Time Using BioFlux Microfluidic Setup

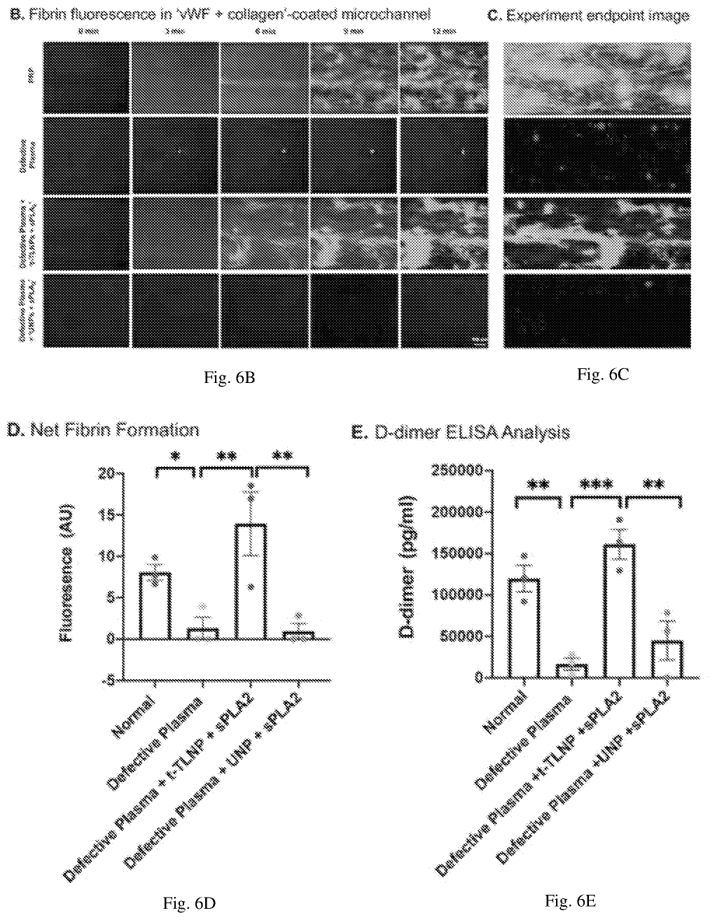

The BioFlux microfluidic setup (Fluxion Biosciences, California, USA) allows a simulation of blood or plasma flow at physiological and pathological shear rates over bioactive-molecule-coated microchannels, using a customized flow controller system, and the channels can be imaged in real time using a fluorescence microscope to assess specific cellular and molecular processes (FIG. 6A). This system was previously described in the assessment of “VBP+CBP”-decorated (i.e., targeted) vs control particle adhesion under a 25 dyn/cm² shear stress flow on “vWF+collagen”-coated microchannels. Since t-TLNPs showed enhanced binding on the “vWF+collagen”-coated surface and also showed the capability of restoring/improving fibrin generation as well as ROTEM-based clot characteristics in anticoagulant-treated and platelet-depleted plasma and blood samples (results from FIGS. 4 and 5), we sought to study whether these capabilities of t-TLNPs enable enhanced fibrin generation/restoration in hemostatically defective plasma under a simulated vascular flow environment on “VWF+collagen”-coated microchannels in the BioFlux setup. For these studies, the hemostatic defect was induced by a combination of “Apixaban anticoagulation+platelet depletion” in plasma, to render a drastic debilitation in endogenous thrombin generation capability of the plasma, which in turn would render a drastic reduction in fibrin. The treatment comparison was done between “t-TLNP+sPLA₂” vs “UNP+sPLA₂” to simulate the injury-site-relevant enzyme-triggered payload release.

The platelets in the plasma were prestained with calcein, and the plasma was also spiked with AlexaFluor-647-labeled fluorescent fibrinogen, such that the platelet accumulation and fibrin formation on the “vWF+collagen”-coated microchannel surface can be imaged under a fluorescence microscope. The flow experiments were maintained for 12 min, and the end point surface-averaged fibrin fluorescence intensity was analyzed. Following this the plasma flow was stopped, the channels were washed with a flow of saline, and the fibrin that formed within the channels was digested with plasmin to be quantified by a D-Dimer assay. FIG. 6B shows representative images of fibrin fluorescence (pseudocolored green) in the channels over 0-12 min for healthy platelet-rich plasma (PRP), “Apixaban-treated+platelet-depleted” plasma (Defect plasma), and treatment effect of this “Defect plasma” with t-TLNPs vs UNPs in the presence of sPLA₂. The representative end point dual-fluorescence (platelets, blue; fibrin, green) images for each of the conditions at the completion of the experiment are shown in FIG. 6C. As is evident from these images, healthy PRP resulted in significant fibrin generation and the end point image showed substantial platelet accumulation within the fibrin mesh. In contrast, “anticoagulation+platelet depletion” resulted in drastic debilitation of fibrin formation in the channel and the end point image showed the accumulation of a small number of platelets and almost no fibrin. When “t-TLNPs+sPLA₂” was added in this drastic defect condition, the fibrin fluorescence was restored over time and the end point image showed significant fibrin formation even though the platelet presence was low. Adding “UNPs+sPLA₂” was unable to have any such fibrin restoration effect, and the images looked similar to those obtained for the defective plasma itself. FIG. 6D shows surface-averaged fluorescence intensity based “net fibrin” quantification data at the experiment end point for each condition tested, and these results emphasized the capability of the “t-TLNPs+sPLA₂” treatment group to significantly restore/improve fibrin formation in the defective plasma. FIG. 6E shows the D-Dimer ELISA based quantification of digested fibrin from the various experiment channel conditions. The D-Dimer concentration is a surrogate measurement for cross-linked fibrin concentration, and these data further corroborated that treatment of defective plasma with “t-TLNPs+sPLA₂” was able to restore fibrin amounts comparable to those of PRP. The D-Dimer concentration for healthy platelet-rich plasma (PRP) was 120000±15900 pg/mL, while that for “Defect plasma” was 16500±7100 pg/mL, indicating a drastic reduction of fibrin generation when endogenous platelet-mediated and FXa-mediated mechanisms of thrombin amplification were reduced. These analyses also indicated that small amounts of fibrin might have formed in the “Defect plasma” channels but were too dispersed and not discernible by fluorescence imaging. In contrast, the D-Dimer concentration for defective plasma channels treated with “t-TLNPs+sPLA₂” was 161000±17800 pg/mL, while that for defective plasma channels treated with “UNPs+sPLA₂” was 45000±23300 pg/mL. This further confirms that sPLA₂-triggered direct release of thrombin from “vWF+collagen” surface-adhered t-TLNPs was able to significantly restore fibrin formation from fibrinogen in “hemostatic defect” settings where endogenous abilities for thrombin generation (and hence fibrin formation) are debilitated due to combined anticoagulation and platelet depletion.

### Prophylactic Dose of t-TLNPs Improve Hemostatic Efficacy in Tail-Clip Bleeding Model in Mice Suffering From Combined Effect of Platelet Depletion and Anti-coagulation

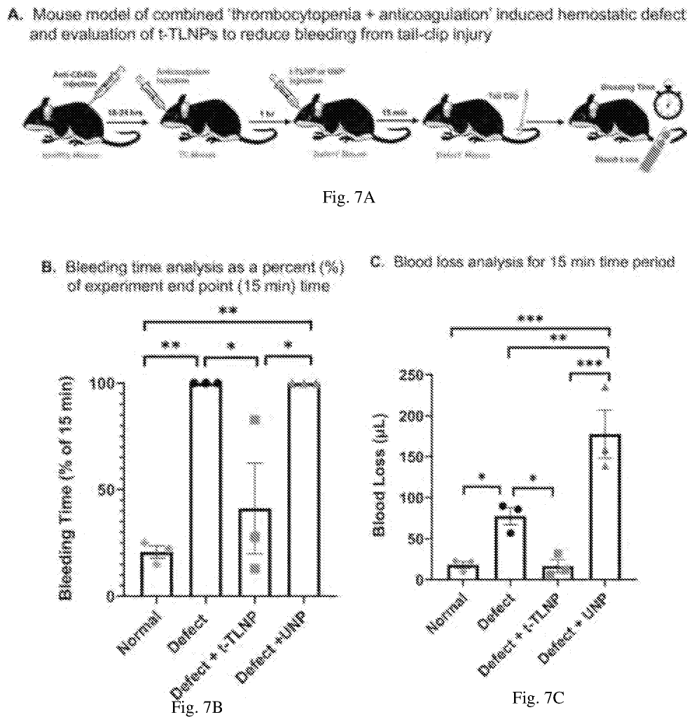

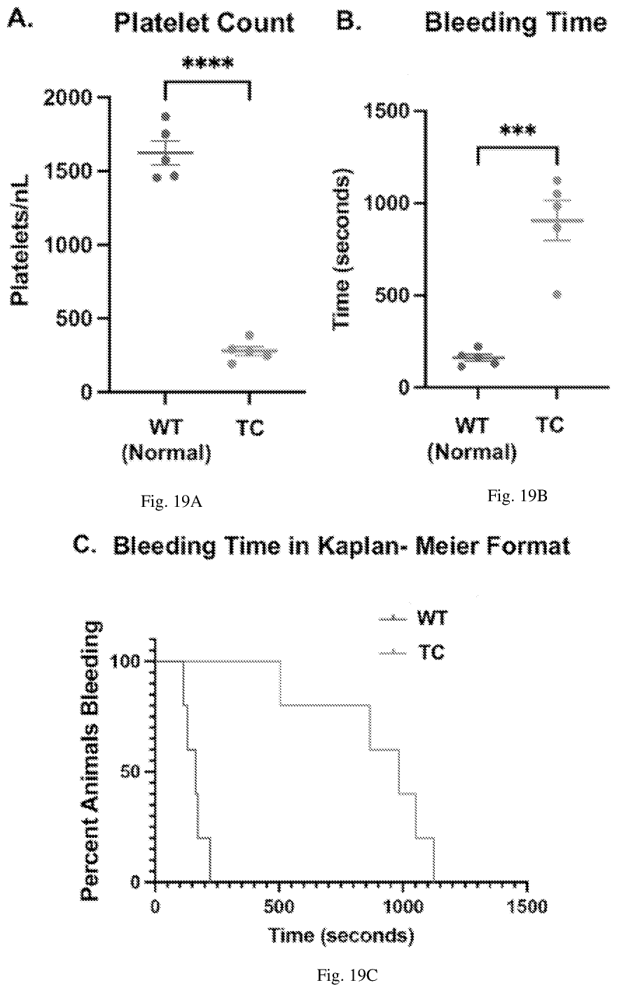

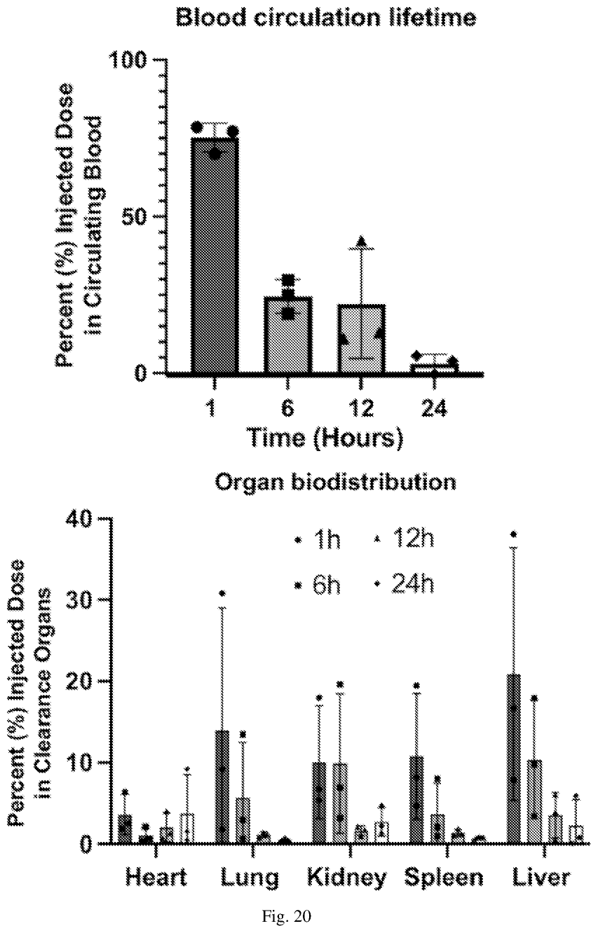

The in vivo feasibility of t-TLNPs was tested in mice using a tail-clip model, where the significant fibrin detriment caused by the combination of platelet depletion and anticoagulation, evident in the in vitro BioFlux studies, was induced in vivo (FIG. 7A). Systemic administration of anti-CD42b (anti-GPIb) antibody can cause transient platelet clearance in mice, leading to a temporary thrombocytopeniatate and impaired hemostatic ability. The tail-clip bleeding model in mice is also a standardized model to study the efficacy of hemostatic agents, where the tail is cut at 1-3 mm from the tip and the time for bleeding to stop as well as the total blood loss in that time is measured. Usually, bleeding from a clipped tail in thrombocytopenia (TC) mice is substantially prolonged in comparison to normal mice but ultimately stops (2-5 min in normal mice vs 15-20 min in thrombocytopenia mice; representative data are shown in FIG. 19). Since our BioFlux studies indicated that combining anticoagulation with platelet depletion drastically reduces fibrin formation, we hypothesized that this “combination effect” would lead to a hemostatic defect in mice more severe than that caused by thrombocytopenia alone and possibly lead to uncontrolled hemorrhage (bleeding does not stop). Before the experiments in mouse tail-clip model were conducted, studies were done to assess the circulation lifetime and biodistribution of t-TLNPs in mice. For this, Rhodamine B (RhB) labeled t-TLNPs were injected retro-orbitally in mice (125 μL injection volume per mouse at 1.14×10¹² particles/mL) and at various time points over 24 h (1, 6, 12, and 24 h) the blood was harvested from the inferior vena cava (IVC), the mouse was sacrificed, and the clearance organs (heart, lung, kidney, spleen, liver) were also harvested and homogenized. The RhB signal in harvested blood as well as the harvested clearance organ homogenate were measured via fluorescence spectrometry and quantified to particle concentration by utilizing a calibration curve of RhB-labeled t-TLNP particle concentrations against the corresponding fluorescence intensity. This enabled the quantification of percent (%) injected dose in blood and clearance organs over the 24 h period. As shown in FIG. 20, the circulation lifetime of the particles in mice was about 24 h, with low accumulation in the clearance organs over time. Of note, the t-TLNP-injected mice did not show any sign of systemic distress, further indicating that the particles are potentially safe in vivo. This allowed subsequent prophylactic administration in the mouse tail-clip model. When mice were administered with anti-CD42b antibody (platelet-depleting antibody) plus low-molecular-weight heparin (LMWH Enoxaparin, anticoagulant), the combined coagulopathic effect led to drastic impairment of hemostasis such that bleeding from clipped tail did not stop at all even beyond 15 min. Therefore, we rationalized that bleeding for 15 min in these “thrombocytopenia+coagulopathic” mice could be considered as the experiment end point to euthanize the mice and the treatment effect of t-TLNPs vs UNPs in restoring hemostatic capability in such mice could be expressed as “percent (%) of 15 min” for which bleeding occurred. Blood loss from the tail clip at 15 min (or earlier if hemostasis occurred) was measured by a spectrophotometric assessment of hemoglobin. FIG. 7B shows the bleeding time (as a percent of 15 min), and FIG. 7C shows blood loss results from these studies.

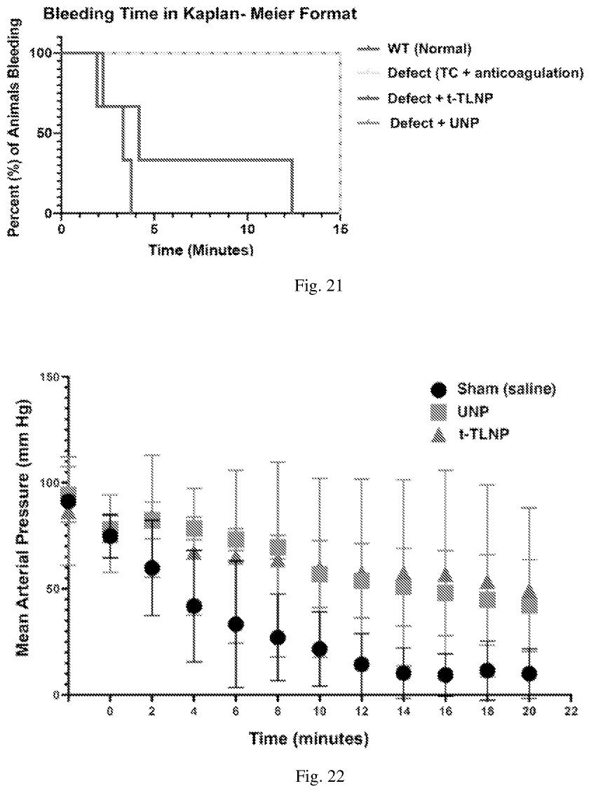

As is evident from FIG. 7B, normal mice stopped bleeding in 3.11±0.44 min, whereas mice administered with anti-CD42b plus Enoxaparin (labeled as “Defect”) continued bleeding at 15 min (and beyond, thus designated as 100% bleeding at 15 min). Treatment with t-TLNPs was able to significantly reduce this bleeding, restoring bleeding cessation at 6.18±3.19 min, whereas treatment with UNPs had no effect (bleeding continued at 15 min and beyond). The bleeding time data for the 15 min time window is shown as a Kaplan-Meier curve in FIG. 21, where the ability of t-TLNPs to significantly reduce bleeding time (the purple line shifting left toward the blue line) is evident. The corresponding blood loss data at the 15 min time point (FIG. 7C) showed that normal mice had a blood loss of 18.06±4.13 μL, whereas mice with a severe hemostatic defect had a significantly increased blood loss of 77±10.41 μL (monitoring stopped at 15 min). Treatment of such “defect” mice with t-TLNPs reduced blood loss to 16.61±7.82 μL, which was comparable to that in normal mice. Interestingly, the treatment of “defect” mice with UNPs further worsened blood loss (177.5±29.47 μL), and this is possibly due to the dilution effect of the administered UNP injection volume which exacerbates coagulopathy without providing any hemostatic benefit. Altogether, these data indicate that in a setting of drastic coagulopathy and hemorrhage where the endogenous thrombin generation (hence fibrin formation) capability is considerably impaired, treatment with t-TLNPs can potentially restore hemostatic capability and reduce bleeding.

### Emergency Dose of t-TLNPs Improve Hemostatic Efficacy in Liver Laceration Bleeding Model in Mice Suffering From Effect of Anticoagulation

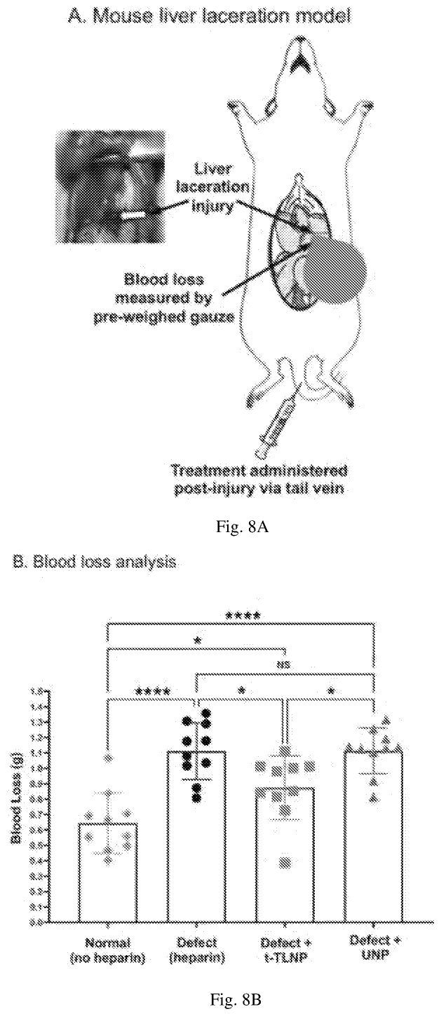

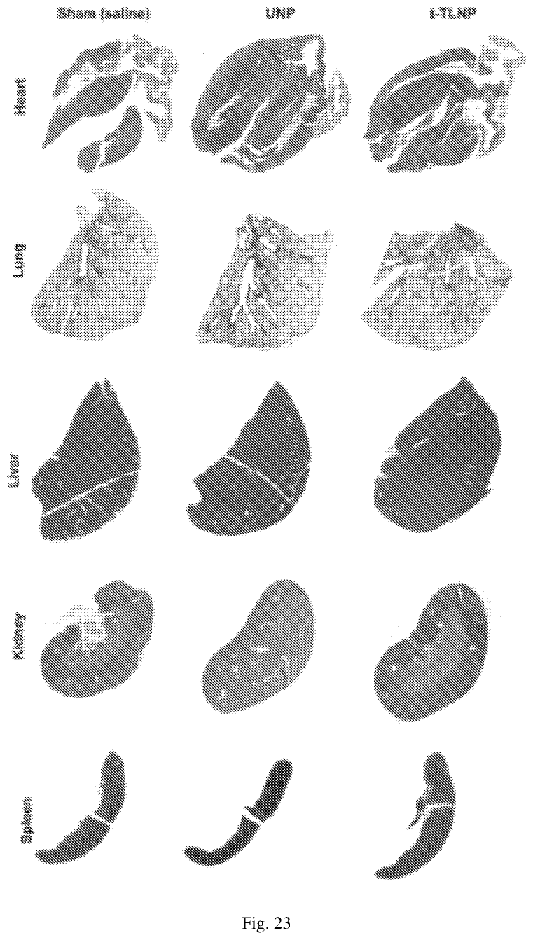

On consideration of the fact that a technology such as t-TLNPs could be potentially used not only for prophylactic management of bleeding risks but also for emergency management of acute hemorrhage (e.g., in trauma), studies were performed in a traumatic liver laceration injury model in mice where the particles were administered intravenously postinjury. In this model, 30 min prior to injury, mice were dosed intravenously with 1 U/g of unfractionated heparin (UFH) to induce a coagulation defect. At the 30 min time point, a midline laparotomy incision was made, preweighed gauze absorption triangles were inserted inside the abdominal cavity of the mouse without touching the liver, and then the right middle lobe of the liver was lacerated and resected, resulting in heavy bleeding in the abdominal cavity. Treatment (sham saline, control UNP, or t-TLNP) was administered via the femoral vein 1 min postinjury (125 μL of injection volume). The abdominal cavity was then stapled close, and the mean arterial pressure (MAP) of the mice was monitored for 20 min. At this 20 min time point the cavity was reopened to retrieve absorption triangles, and the difference between the preinjury and postinjury weight of the absorption triangles was recorded to assess blood loss in grams. FIG. 8A shows the schematic of this experimental setup, with a representative anatomic image of the liver injury site. Following retrieval of the absorption triangles, the abdominal cavity was stapled close again and the mice were kept under observation for 3 h from the time of liver injury. At the 3 h time point the mice were sacrificed, and their clearance organs (uninjured part of liver, heart, lung, kidney, spleen) were harvested for histological staining and imaging with Carstair's stain to assess the off-target clotting risk. FIG. 8B shows the blood loss data from these experiments (n=10 per group). As is evident from the data, heparin treatment resulted in a significant increase in blood loss from the liver injury (normal vs defect) and treatment with UNPs was unable to reduce blood loss in the “defect” mice, whereas treatment with t-TLNPs significantly reduced blood loss in the “defect” mice. FIG. 22 shows the MAP data in “defect” mice from these studies, while FIG. 23 shows representative histology images (Carstair's staining) of the harvested organs postsacrifice. The MAP data show that administration of the UNPs or t-TLNPs did not cause any drastic fluctuation of arterial pressure in comparison to sham (saline treatment), which is indicative of the systemic safety of the particles. The histology data of the organs did not show any sign of clotting in clearance organs, since a fibrin stain (usually bright red in Carstair's stain) was not visible in any of the organs for UNP-treated or t-TLNP-treated mice (histological staining similar to that for saline-treated sham mice). Altogether these results suggest that postinjury emergency administration of t-TLNPs can render targeted hemostatic efficacy while maintaining systemic safety, in an acute traumatic hemorrhage model.

The t-TLNP system actively targets the vascular injury site via peptide-mediated anchorage to vWF and collagen, which mimics the injury site-specific binding mechanisms of our natural hemostatic cells, the platelets. Our microfluidic studies with fluorescently labeled “VBP+CBP”-decorated particles flowed over a “vWF+collagen”-coated surface vs albumin-coated surface confirmed this specific anchorage ability.

Additionally, our biosafety assays confirmed that the “VBP+CBP”-decorated particles do not have unwanted activation of healthy endothelial cells, resting platelets, neutrophils, and complement C3, suggesting that the particles would be physiologically safe in vivo. Our studies also showed that the diffusive release of the particle-encapsulated payload was very low, and the shear-induced release of the payload was also very low, in comparison to the significantly higher release of the payload upon particle destabilization by the enzyme sPLA₂ (predominantly due to hydrolysis of sn-2 ester bond of phospholipids in the liposome membrane lipids). Such phospholipase enzymes have been reported to be upregulated at vascular injury sites due to production from activated platelets, and therefore we rationalize that injury-site-anchored t-TLNPs can be amenable to sPLA₂-triggered enhanced release of encapsulated thrombin. For our in vitro and in vivo studies the t-TLNPs were used to render a thrombin concentration of 1-2.5 nM, which is at the lower end of what is needed to render fibrin formation. In circulation there are thrombin inhibitors such as antithrombin and α-macroglobulin that can neutralize thrombin rapidly in circulation, but thrombin is protected from such inhibition at the injury site when it associates with fibrin. Therefore, a low fraction of the 1-2.5 nM of thrombin that may leak out from the t-TLNPs by diffusion is expected to be quickly neutralized in circulation, but when the particle-encapsulated thrombin is released at higher amounts due to “diffusion+sPLA₂-triggered particle degradation” at the injury site from site-localized t-TLNPs, this thrombin will be protected from inhibition by the fibrin forming at the site and will continue to augment hemostasis.

Our in vitro studies utilizing a spectrophotometric assessment of fibrin generation in human plasma and a ROTEM-based assessment of clot kinetics and viscoelastic properties in human blood indicated that thrombin released from t-TLNPs can indeed restore fibrin generation and the corresponding clot characteristics when the endogenous thrombin (and hence fibrin) generation capability is drastically affected due to anticoagulation or platelet depletion. Therefore, we combined these two detrimental effects (anticoagulation+platelet depletion) to render a drastic coagulopathy condition in human plasma, and our BioFlux-based in vitro microfluidic studies indicated that, even under such severe hemostatic impairment condition, t-TLNPs can restore fibrin generation and polymerization comparable to those of the positive control of healthy PRP. Subsequently, we created this severe hemostatic impairment in vivo in mice systemically administered with a combination of antiplatelet antibody and anticoagulant agent, and these mice showed incessant bleeding from a tail-clip injury for over 15 min. Intravenous treatment with t-TLNPs prophylactically administered in these mice was able to render improved hemostasis and significantly reduced blood loss. Additional in vivo studies using a liver laceration acute hemorrhage model in heparinized mice showed that postinjury intravenous administration of t-TLNPs could significantly reduce blood loss. In hemodynamic observations (e.g., of MAP) as well as posteuthanasia histology analyses of clearance organs, no indication of off-target clotting was found. Therefore, considering the fact that our studies showed a t-TLNP circulation lifetime of about 24 h in mice, one can envision use of such a technology in both prophylactic and emergency settings. Altogether, our in vitro and in vivo studies provide exciting evidence for t-TLNPs as an effective platelet-inspired intravenous hemostatic nanomedicine.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

1. A biosynthetic hemostat comprising:
a biocompatible flexible nanoparticle that includes a shell that defines an outer surface of the nanoparticle and a core, which is loaded with thrombin, and a plurality of von Willebrand factor-binding peptides (VBPs) and collagen-binding peptides (CBPs) that are linked to the shell and extend from the outer surface, wherein the nanoparticle is configured to adhere to a vascular surface, vascular disease site and/or vascular injury site with exposed von Willebrand factor (vWF) and collagen, shield the loaded thrombin in circulation to avoid rapid inhibition or systemic thrombotic risk, and release the loaded thrombin at the vascular surface, vascular disease site and/or vascular injury site by diffusion from the nanoparticle and/or enzyme triggered degradation of the nanoparticle.

2. The hemostat of claim 1, wherein the nanoparticle binds to the vascular surface, vascular disease site and/or vascular injury site under a hemodynamic shear environment.

3. The hemostat of claim 1, wherein the enzyme that triggers degradation of the nanoparticle is substantially unique or specific to the vascular disease site and/or vascular injury site and/or has a higher concentration or activity at the vascular disease site and/or vascular injury site compared to other cells, tissues, and/or disease sites in the subject.

4. The hemostat of claim 1, wherein the shell of the nanoparticle includes at least one phospholipid and the enzyme includes at least one phospholipase that triggers phospholipase degradation of the at least on phospholipid and release of the thrombin.

5. The hemostat of claim 1, wherein the nanoparticle has a diameter of about 50 nm to about 5 μm.

6. The hemostat of claim 1, wherein the nanoparticle is a liposome.

7. The hemostat of claim 6, wherein the liposome includes a plurality of phospholipids and optionally cholesterol to define a lipid membrane.

8. The hemostat of claim 7, wherein the phospholipids include ate least one of distearoylphosphatidylserine (DSPS), distearoylphosphatidylcholine dipalmitoylphosphatidylcholine (DSPC), dibehenoylglycerophosphocoline (DBPC), distearoylphosphatidylcholine (DSPC), diarachidonylphosphatidylcholine (DAPC), dioleoylphosphatidylethanolamine (DOPE), dipalmitoylphosphatidylethanolamine (DPPE), and distearoylphosphatidylethanolamine (DSPE); dipalmitoylphosphatidic acid (DPPA), or polyethylene glycol (PEG) functionalized lipids thereof.

9. The hemostat of claim 7, wherein the VBPs and CBPs are conjugated to the phospholipids with PEG linkers.

10. The hemostat of claim 9, wherein the VBP and CBP conjugated phospholipids comprise about 1 mole % to about 10 mole %, of the total lipid composition of the liposome.

11. The hemostat of claim 6, wherein the liposome comprises DSPC, DSPE conjugated to VBP with PEG (DSPE-PEG-VBP), DSPE conjugated to CBP with PEG (DSPE-PEG-CBP), and cholesterol.

12. The hemostat of claim 1, wherein the VBPs and CBPs are spatially or topographically arranged on the outer surface such that the VBPs and CBPs do not spatially mask each other and the nanoparticle is able to adhere to the vascular surface, vascular disease site, and/or vascular injury site with exposed vWF and collagen and promote arrest and aggregation of active platelets onto sites of the nanoparticle adhesion.

13. The hemostat of claim 1, wherein the nanoparticle has shape, size and elastic modulus that facilitates margination to a vascular injury site upon administration to vasculature of a subject.

14. The hemostat of claim 1, the VBPs have an amino acid sequence of SEQ ID NO: 1 and the CBPs have an amino acid sequence of SEQ ID NO: 2.

15. (canceled)

16. The hemostat of claim 1, wherein the nanoparticle further includes a plurality of fibrinogen mimetic peptides (FMPs) that bind to GPIb-IIIa, endothelial cell targeting peptides, and/or platelet targeting peptides that are linked to the shell and extend from the outer surface.

17. (canceled)

18. The hemostat of claim 1, comprising a plurality of the nanoparticles, wherein upon administration to a subject the hemostat provides vascular injury-site targeted delivery of thrombin.

19. The hemostat of claim 18, wherein the amount of thrombin delivered to the vascular injury site in the subject by the hemostat is an amount effective to augment hemostasis in the subject.

20. The hemostat of claim 18, wherein the plurality of nanoparticles are further loaded with at least one of a platelet agonist, antifibrinolytic, coagulation factor, or prothrombin.

21. (canceled)

22. A method treating at least one of a non-compressible and/or uncontrolled hemorrhage, trauma-induced coagulopathy, congenital, disease associated, or drug induced hemostatic dysfunction, or systemic bleeding dysfunction including polytrauma, internal bleeding, platelet and coagulation factor defects in a subject in need thereof, the method comprising:
administering to the subject a therapeutically effective amount of the hemostat of claim 1.

23-30. (canceled)

31. A kit comprising the biosynthetic hemostat of claim 1 and optionally fibrinogen and calcium, wherein biosynthetic hemostat and optional fibrinogen if present are stored in separate compartments.

32. (canceled)
